(19) United States
(12) Patent Application Publication
(10) Pub. No.: US 2023/0093169 A1
(43) Pub. Date: Mar. 23, 2023

(54) COMBINATIONS OF ANTIBODY CONSTRUCTS AND INHIBITORS OF CYTOKINE RELEASE SYNDROME AND USES THEREOF

(71) Applicants: AMGEN RESEARCH (MUNCH) GMBH, Munich (DE); AMGEN INC., Thousand Oaks, CA (US)

(72) Inventors: Benno Rattel, Munich (DE); Matthias Friedrich, Munich (DE); Oliver Thomas, Munich (DE); Tara Arvedson, Thousand Oaks, CA (US); Jackson Egen, Thousand Oaks, CA (US); Jason DeVoss, Thousand Oaks, CA (US); Xiaoting Wang, Thousand Oaks, CA (US); Grit Lorenczewski, Munich (DE)

(21) Appl. No.: 17/793,757

(22) PCT Filed: Jan. 22, 2021
(86) PCT No.: PCT/US2021/014517

§ 371 Date: Jul. 19, 2022

Related U.S. Application Data
(60) Provisional application No. 62/964,324, filed on Jan. 22, 2020.

(51) Int. Cl. C07K 16/28; A61P 35/00; A61K 31/573; A61K 39/395; A61P 37/06; A61K 38/17; C07K 16/24

(52) U.S. Cl. CPC C07K 16/2809; A61P 35/00; A61K 31/573; A61K 39/3955; A61P 37/06; A61K 39/39558; A61K 38/1793; C07K 16/241; C07K 16/2803; A61K 2039/507

(57) ABSTRACT

The present invention relates to medical combination products comprising (i) at least one antibody construct comprising at least one domain which binds to a target antigen expressed on the surface of a cell and at least one other domain which binds to CD3 as well as (ii) at least one molecule that is an antagonist of/an inhibitor of signaling, which is based on an interaction of TNF with its cognate receptor (TNFR), wherein the antagonisation or the inhibition of TNF or its cognate receptor prevents, reduces, or blocks TNF/TNFR mediated signalling. Furthermore, the invention provides therapeutic and preventive methods and medical uses of said combination products, as well as a kit comprising said at least one antibody construct and at least one antagonist/inhibitor of TNF or its cognate receptor, wherein the interaction of said antagonist/inhibitor of TNF with its cognate receptor reduces, mitigates, prevents, or treats cytokine release syndrome.

The present invention relates to medical uses of (i) antibody constructs comprising at least one domain which binds to CD3 on a T cell and at least one other domain that binds a target on a target cell in combinations with (ii) inhibitors/antagonists of TNF-alpha (TNF) and/or TNF-alpha-Receptor (TNFR), thereby reducing or blocking signalling that is based on a TNF/TNFR interaction. The pharmaceutical combination products or kits comprising the above inhibitors/antagonists and CD3-binding antibody constructs disclosed herein may be used in the treatment, prevention or mitigation of neoplastic cell growth or cancer and block the development of cytokine release syndrome (CRS) or mitigate, ameliorate or treat symptoms associated with excessive release of cytokines, particularly with CRS.

### BACKGROUND OF THE INVENTION

Recently, immunotherapeutic approaches in the treatment, prevention, mitigation of neoplastic cell growth in cancer patients have witnessed impressive progress. For example, the CD19-directed bispecific T-cell engager (BiTE®) molecule Blinatumumab was approved in 2014 for Philadelphia chromosome-negative relapsed or refractory B-cell precursor ALL under the FDA's accelerated approval program. Bispecific antibody constructs comprising one domain that binds to CD3 on T cells and another domain that binds to a protein expressed on target cells directly connect T cells to target cells to induce T cell redirected lysis. This mechanism of action is distinct from chemotherapy and other immunotherapy in that it can work with any CD3-positive T cell, independent of a costimulatory activating signal (Klinger et al., Immunol Reviews 2016). However, occasionally patients that have been treated with effector molecules that comprise a domain binding CD3 are associated with adverse events, for example, due to an excessive activation of the immune system resulting in the release of proinflammatory cytokines. The release of these cytokines may cause milder adverse events, such as mild headaches. In rare events, patients may develop cytokine release syndrome (CRS). CRS can present with a variety of symptoms ranging from mild, flu-like symptoms to severe life-threatening manifestations of the excessive inflammatory response. Mild symptoms of CRS include fever, fatigue, headache, rashes, arthralgia, and myalgia. More severe cases are characterized by hypotension as well as high fever and can progress to an uncontrolled systems inflammatory response with vasopressor-requiring circulatory shock, vascular leakage, disseminated intravascular coagulation, and multi-organ system failure. Laboratory abnormalities that are common in patients with CRS include cytopenias, elevated creatinine and liver enzymes, deranged coagulation parameters, and a high CRP (Shimabukuro-Vornhagen et al., J. ImmunoTher Cancer (2018) 6:56). These adverse events are not only seen in patients treated with BiTE® molecules, but also in patients who have been administered with monoclonal antibodies, chimeric antigen receptor (CAR) T-cells, and tyrosine kinase inhibitors (Riegler et al., Therapeutics and Clinical Risk Management 2019: 15, 323-335).

The FDA recently approved tocilizumab, a monoclonal antibody that competes with the binding of the proinflammatory cytokine IL-6 to its receptor, IL-6R, thereby inhibiting IL-6R signalling in effector cells, for the treatment of severe or life-threatening CAR T-cell-induced CRS in adults and pediatric patients that are older than 2 years. However, tocilizumab is not recommended for the primary management of neurotoxicity, one possible adverse event associated with immunotherapy (e.g., administration of T cell engaging target-specific immunoglobulin-based constructs) and some patients do not respond to tocilizumab. In addition, tocilizumab might increase the risk of long-term immunosuppression and repetitive administration of tocilizumab to patients with rheumatic diseases has resulted in a higher incidence of lower intestinal perforations, which may not occur in the acute setting (Borrega et al., HemaSphere (2019) 3:2 (e19).

Other immunomodulatory agents that have been used or suggested for the treatment of CRS relate to very specific applications. For example, the anti-IL-6 antibody siltuximab, T cell depleting antibody alemtuzumab and Anti-Thymocyte Globulin (ATG), IL-1-based inhibitors (anakinra), cyclophosphamide, ibrutinib, and GM-CSF inhibition or cytokine adsorption have been suggested (Borrega et al., HemaSphere (2019) 3:2 (e19)). TNF-alpha (hereinafter referred to simply as “TNF” in view of the old misleading and no longer used designation “TNF-beta” for the cytokine “lymphotoxin” as suggested, inter alia, by I. A. Clark/Cytokine & Growth Factor Reviews 18 (2007) 335-343), another player in the CRS development has also been discussed. TNF inhibitors/antagonists such as etanercept and infliximab have been used with mixed results in treating severe CRS (Riegler et al., Therapeutics and Clinical Risk Management 2019: 15, 323-335; Li et al., Sci Transl Med 11, eaax8861 (2019)).

Approaches have been made to prevent, reduce or treat adverse events associated with the administration of antibody constructs comprising a T cell engager domain, e.g., a CD3 binding domain, by co-administration with glucocorticoids, for example with dexamethasone. Corticosteroids have also been used in the treatment of CAR T-cell therapy-associated CRS but results of clinical trials are divergent (Borrega et al., HemaSphere (2019) 3:2 (e19)). The general picture concerning the treatment of CRS using different immunotherapeutic approaches is therefore unclear. While in a given therapeutic setting, a specific inhibitor or modulator may be effective, other immunotherapeutic approaches that may provoke or did result in CRS may require different approaches. Accordingly, there is a persistent need for new immunotherapies in which CRS is prevented, reduced, mitigated and/or treated.

Further, there is also a need to provide new preventive and therapeutic measures against adverse events associated with the administration of antibody constructs comprising a T cell engager domain, e.g., a CD3 binding domain, particularly for cancer patients having a corticosteroid intolerance, for example a dexamethasone intolerance, or particularly for cancer that are at risk of developing adverse events against corticosteroids, such as dexamethasone. Similarly, there is a need to provide new preventive measures against adverse events associated with the administration of antibody constructs comprising a T cell engager domain, e.g., a CD3 binding domain, particularly for cancer patients with an intolerance against IL-6 antagonists or IL-6R-antagonists, for example particularly for cancer with a tocilizumab intolerance, or cancer patients that are at risk of developing adverse events against such IL-6 antagonists or IL-6R-antagonists, for example a tocilizumab, e.g., for those cancer patients that have developed autoantibodies against tocilizumab. These and other problems are solved by the present invention.

### DETAILED DESCRIPTION OF THE INVENTION

The present invention relates, inter alia, to the following aspects and embodiments as well as to all combinations of these embodiments provided no scientific or technical reasons exist that render such combination unfeasible:

### Aspect A)—Combination Products

(i) A combination product for the prevention or reduction of adverse events associated with immunotherapy, particularly cancer immunotherapy, with an antibody construct comprising at least one domain (also referred to as “first domain”) which binds to a target antigen on the surface of a target cell, and at least one other domain (also referred to as “second domain”) which binds to human CD3 on the surface of a T cell, comprising
   (a) at least one antibody construct referred to in the preamble, and
   (b) an inhibitor/antagonist of TNF/TNFR that reduces TNF/TNFR signalling,
wherein said inhibitor/antagonist of TNF/TNFR that reduces TNF/TNFR signalling referred to in (b) is for administration to a patient prior to administration of said antibody construct referred to in (a), particularly wherein the patient is not a rodent, and more particularly, wherein the patient is either a human being or a non-human primate
(ii) The combination product according to embodiment (i) for the prevention or reduction of adverse events associated with immunotherapy with an antibody construct comprising a first domain which binds to a target antigen on the surface of a target cell, and a second domain which binds to human CD3 on the surface of a T cell, wherein the adverse event associated with immunotherapy is increased cytokine release of TNF, IL-1, MCP-1, and/or IL-6, particularly wherein the adverse event is cytokine release syndrome (CRS) or tumor lysis syndrome (TLS). Adverse events include also a neurological reaction, preferably one or more selected from the group consisting of: confusion, ataxia, disorientation, dysphasia, aphasia, speech impairment, cerebellar symptoms, tremor, apraxia, seizure, grand mal convulsion, palsy, and balance disorder.
(iii) The combination product according to embodiment (i) or (ii) for the prevention or reduction of adverse events associated with immunotherapy with an antibody construct comprising a first domain which binds to a target antigen on the surface of a target cell, and a second domain which binds to human CD3 on the surface of a T cell, wherein the second domain of said antibody construct binds to human CD3 epsilon and to Callithrix jacchus or Saimiri sciureus CD3 epsilon.
(iv) The combination product according to embodiment (i) to (iii) for the prevention or reduction of adverse events associated with immunotherapy with an antibody construct comprising a first domain which binds to a target antigen on the surface of a target cell, and a second domain which binds to human CD3 on the surface of a T cell, wherein
   a) the antibody construct is a single chain antibody construct,
   b) the first domain is in the format of an scFv,
   c) the second domain is in the format of an scFv,
   d) the first and the second domain are connected via a linker, and/or
   e) the antibody construct comprises a domain providing an extended serum half-life.
(v) The combination product according to any one of the preceding embodiments for the prevention or reduction of adverse events associated with immunotherapy with an antibody construct comprising a first domain which binds to a target antigen on the surface of a target cell, and a second domain which binds to human CD3 on the surface of a T cell, wherein the first domain binds to a target antigen selected from the group comprising tumor-associated antigens, viral antigens, bacterial antigens, peptide-MHC complexes presenting a peptide fragments derived from a tumor-associated antigen, viral antigen, bacterial antigen, or a disease-associated antigen.
(vi) The combination product according to any of the preceding embodiments for the prevention or reduction of adverse events associated with immunotherapy of a disease with an antibody construct comprising a first domain which binds to a target antigen on the surface of a target cell, and a second domain which binds to human CD3 on the surface of a T cell, wherein said disease is a neoplastic disease.
(vii) The combination product according to any one of the preceding embodiments for the prevention or reduction of adverse events associated with immunotherapy with an antibody construct comprising a first domain which binds to a target antigen on the surface of a target cell, and a second domain which binds to human CD3 on the surface of a T cell, wherein the inhibitor/antagonist of TNF/TNFR that reduces TNF/TNFR signalling is selected from the group comprising small molecules, biological molecules, antibodies and derivatives thereof, aptamers, etc.
(viii) The combination product according to any one of the preceding embodiments for the prevention or reduction of adverse events associated with immunotherapy with an antibody construct comprising a first domain which binds to a target antigen on the surface of a target cell, and an inhibitor/antagonist of TNF/TNFR that reduces TNF/TNFR signalling is selected from the group of TNF inhibitors comprising etanercept, infliximab, adalimumab, certolizumab Pergol, and golimumab, particularly etanercept.
(ix) The combination product according to any one of the preceding embodiments for the prevention or reduction of adverse events associated with immunotherapy with an antibody construct comprising a first domain which binds to a target antigen on the surface of a target cell, and a second domain which binds to human CD3 on the surface of a T cell, wherein the first domain binds to a target antigen, wherein said target antigen is a tumor-associated antigen that is expressed or present on solid tumors or hematological tumors selected from the group of antigens comprising CD19, CD33, FLT3, PSMA, BCMA, and DLL3.
(x) The combination product according to any of the preceding embodiments for the prevention or reduction of adverse events associated with immunotherapy with an antibody construct comprising a first domain which binds to a target antigen on the surface of a target cell, and a second domain which binds to human CD3 on the surface of a T cell, wherein said combination product is for administration to a patient at risk of developing adverse events or having an intolerance to at least one of a group comprising corticosteroids, IL-6-inhibitors, IL-6R-inhibitors, and/or TNF/TNFR inhibitors different from an inhibitor/antagonist of TNF/TNFR that reduces TNF/TNFR signalling according to any of the preceding embodiments.
(xi) The combination product according to any of the preceding embodiments, particularly embodiment (viii), wherein the inhibitor/antagonist of TNF/TNFR that reduces TNF/TNFR signalling etanercept is particularly preferred, for the prevention or reduction of adverse events associated with immunotherapy with an antibody construct comprising a first domain which binds to a target antigen on the surface of a target cell, and a second domain which binds to human CD3 on the surface of a T cell, wherein said combination product is for administration to a patient at risk of developing adverse events or having an intolerance to corticosteroids, wherein the corticosteroid is dexamethasone.
(xii) The combination product according to any of the preceding embodiments for the prevention or reduction of adverse events associated with immunotherapy with an antibody construct comprising a first domain which binds to a target antigen on the surface of a target cell, and a second domain which binds to human CD3 on the surface of a T cell, wherein said combination product is for administration to a patient at risk of developing adverse events or having an intolerance to IL-6-antagonists and/or IL-6R-antagonists, wherein the IL-6-antagonists and/or IL-6R-antagonists are selected from the group comprising tocilizumab, siltuximab, olokizumab, elsilimomab, clazakizumab, sirukumab, particularly tocilizumab, and/or wherein said combination product is for administration to a patient at risk of developing adverse events or having an intolerance to TNF/TNFR inhibitors, wherein the TNF/TNFR inhibitor is selected from the group comprising infliximab, adalimumab, certolizumab Pergol, and/or golimumab.
(xiii) The combination product according to any of embodiments (i) to (x) and (xii) for the prevention or reduction of adverse events associated with immunotherapy of a disease with an antibody construct comprising a first domain which binds to a target antigen on the surface of a target cell, and a second domain which binds to human CD3 on the surface of a T cell, preferably a neoplastic disease, wherein said combination product further comprises at least one corticosteroid.
(xiv) The combination product according to any of embodiments (i) to (x) and (xiii) for the prevention or reduction of adverse events associated with immunotherapy of a disease with an antibody construct comprising a first domain which binds to a target antigen on the surface of a target cell, and a second domain which binds to human CD3 on the surface of a T cell, preferably a neoplastic disease, said combination product further comprises at least one corticosteroid, wherein said corticosteroid is dexamethasone, particularly embodiment (viii), wherein as the inhibitor/antagonist of TNF/TNFR that reduces TNF/TNFR signalling etanercept is particularly preferred.
(xv) The combination product according to any of embodiments (i) to (xi), (xiii), and (xiv) for the prevention or reduction of adverse events associated with immunotherapy of a disease with an antibody construct comprising a first domain which binds to a target antigen on the surface of a target cell, and a second domain which binds to human CD3 on the surface of a T cell, preferably for a neoplastic disease, wherein said combination product further comprises an IL-6R-antagonist, wherein said IL-6R-antagonist is tocilizumab.
(xvi) The combination product according to any of embodiments (i) to (ix) for the prevention or reduction of adverse events associated with immunotherapy of a disease, preferably a neoplastic disease, with an antibody construct comprising a first domain which binds to a target antigen on the surface of a target cell, and a second domain which binds to human CD3 on the surface of a T cell, further comprising at least one corticosteroid, particularly dexamethasone and/or at least one IL-6 and/or IL-6R-antagonist, particularly tocilizumab, wherein said at least one corticosteroid and/or said IL-6 and/or IL-6R-antagonist is for administration to a patient in need thereof prior to, concomitant with, and/or after administration of said antibody construct (a).

### Aspect B)—Methods of Prevention

(i) A method of for the prevention or reduction of adverse events associated with immunotherapy, preferably immunotherapy of a neoplastic disease, with an antibody construct comprising a first domain which binds to a target antigen on the surface of a target cell, and a second domain which binds to human CD3 on the surface of a T cell, comprising administering a combination product of any one of the preceding embodiments of Aspect A) to a patient in need thereof, wherein said patient is a human being or a non-human primate.
(ii) The method for the prevention or reduction of adverse events associated with immunotherapy with an antibody construct comprising a first domain which binds to a target antigen on the surface of a target cell, and a second domain which binds to human CD3 on the surface of a T cell according to embodiment (i) of Aspect B) comprising
   (a) administering an antibody construct comprising a first domain which binds to a target antigen on the surface of a target cell, and a second domain which binds to human CD3 on the surface of a T cell,
   (b) administering an inhibitor/antagonist of TNF/TNFR that reduces TNF/TNFR signalling,
wherein said inhibitor/antagonist of TNF/TNFR that reduces TNF/TNFR signalling (b) is administered to the patient prior to administration of said antibody construct (a).
(iii) The method for the prevention or reduction of adverse events associated with immunotherapy with an antibody construct comprising a first domain which binds to a target antigen on the surface of a target cell, and a second domain which binds to human CD3 on the surface of a T cell according to any one of embodiments (i) and (ii) of Aspect B), wherein the adverse event associated with immunotherapy is CRS or TLS. Adverse events include, but are not limited to a neurological reaction, preferably one or more selected from the group consisting of: confusion, ataxia, disorientation, dysphasia, aphasia, speech impairment, cerebellar symptoms, tremor, apraxia, seizure, grand mal convulsion, palsy, and balance disorder.
(iv) The method for the prevention or reduction of adverse events associated with immunotherapy with an antibody construct comprising a first domain which binds to a target antigen on the surface of a target cell, and a second domain which binds to human CD3 on the surface of a T cell according to embodiments (i) to (iii) of Aspect B), wherein the second domain of said antibody construct binds to human CD3 epsilon and to Callithrix jacchus or Saimiri sciureus CD3 epsilon.
(v) The method for the prevention or reduction of adverse events associated with immunotherapy with an antibody construct comprising a first domain which binds to a target antigen on the surface of a target cell, and a second domain which binds to human CD3 on the surface of a T cell according to embodiments (i) to (iv) of Aspect B), wherein
   a) the antibody construct is a single chain antibody construct,
   b) the first domain is in the format of an scFv,
   c) the second domain is in the format of an scFv,
   d) the first and the second domain are connected via a linker, and/or
   e) the antibody construct comprises a domain providing an extended serum half-life.
(vi) The method for the prevention or reduction of adverse events associated with immunotherapy with an antibody construct comprising a first domain which binds to a target antigen on the surface of a target cell, and a second domain which binds to human CD3 on the surface of a T cell according to embodiments (i) to (v) of Aspect B), wherein the first domain binds to a target antigen selected from the group comprising tumor-associated antigens, viral antigens, bacterial antigens, peptide-MHC complexes presenting a peptide fragments derived from a tumor-associated antigen, viral antigen, bacterial antigen, or a disease-associated antigen.
(vii) The method for the prevention or reduction of adverse events associated with immunotherapy with an antibody construct comprising a first domain which binds to a target antigen on the surface of a target cell, and a second domain which binds to human CD3 on the surface of a T cell according to embodiments (i) to (vi) of Aspect B), wherein the first domain binds to a target antigen, wherein said target antigen is a tumor-associated antigen selected from the group comprising CD19, CD33, FLT3, PSMA, BCMA, and DLL3.
(viii) The method for the prevention or reduction of adverse events associated with immunotherapy with an antibody construct comprising a first domain which binds to a target antigen on the surface of a target cell, and a second domain which binds to human CD3 on the surface of a T cell according to embodiments (i) to (vii) of Aspect B), wherein the antagonist of TNF/TNFR that reduces TNF/TNFR signalling is selected from the group comprising small molecules, biological molecules, aptamers, antibodies and derivatives thereof.
(ix) The method for the prevention or reduction of adverse events associated with immunotherapy with an antibody construct comprising a first domain which binds to a target antigen on the surface of a target cell, and a second domain which binds to human CD3 on the surface of a T cell according to embodiments (i) to (viii) of Aspect B), wherein the inhibitor/antagonist of TNF/TNFR that reduces TNF/TNFR signalling is selected from the group of TNF inhibitors/antagonists comprising etanercept, infliximab, adalimumab, certolizumab Pergol, and golimumab, particularly etanercept.
(x) The method for the prevention or reduction of adverse events associated with immunotherapy of a disease with an antibody construct comprising a first domain which binds to a target antigen on the surface of a target cell, and a second domain which binds to human CD3 on the surface of a T cell according to embodiments (i) to (ix) of Aspect B), wherein said disease is a neoplastic disease.
(xi) The method for the prevention or reduction of adverse events associated with immunotherapy with an antibody construct comprising a first domain which binds to a target antigen on the surface of a target cell, and a second domain which binds to human CD3 on the surface of a T cell according to embodiments (i) to (x) of Aspect B), wherein said combination product is for administration to a patient at risk of developing adverse events or having an intolerance to one of a group comprising corticosteroids, IL-6-inhibitors, IL-6R-inhibitors, and/or inhibitor/antagonist of TNF/TNFR that are different from the group of inhibitors/antagonists of TNF/TNFR according to (b) in any one of the preceding embodiments, particularly different from etanercept.
(xii) The method for the prevention or reduction of adverse events associated with immunotherapy with an antibody construct comprising a first domain which binds to a target antigen on the surface of a target cell, and a second domain which binds to human CD3 on the surface of a T cell according to embodiments (i) to (x) of Aspect B), wherein said combination product is for administration to a patient at risk of developing adverse events or having an intolerance to corticosteroids, wherein the corticosteroid is dexamethasone.
(xiii) The method for the prevention or reduction of adverse events associated with immunotherapy with an antibody construct comprising a first domain which binds to a target antigen on the surface of a target cell, and a second domain which binds to human CD3 on the surface of a T cell according to embodiments (i) to (x) and (xii) of Aspect B), preferably in the immunotherapy of a neoplastic disease, wherein said combination product is for administration to a patient at risk of developing adverse events or having an intolerance to IL6-antagonists and/or IL-6R-antagonists, particularly wherein the IL-6R-antagonist is tocilizumab.
(xiv) The method for the prevention or reduction of adverse events associated with immunotherapy with an antibody construct comprising a first domain which binds to a target antigen on the surface of a target cell, and a second domain which binds to human CD3 on the surface of a T cell according to embodiments (i) to (xi) and (xiii) of Aspect B), preferably immunotherapy of a neoplastic disease, wherein said combination product further comprises at least one corticosteroid.
(xv) The method for the prevention or reduction of adverse events associated with immunotherapy with an antibody construct comprising a first domain which binds to a target antigen on the surface of a target cell, and a second domain which binds to human CD3 on the surface of a T cell according to embodiments (i) to (xi) and (xiii) of Aspect B), preferably in the immunotherapy of a neoplastic disease, wherein said corticosteroid is dexamethasone, particularly embodiment wherein as the inhibitor/antagonist of TNF/TNFR that reduces TNF/TNFR signalling etanercept is particularly preferred.
(xvi) The method for the prevention or reduction of adverse events associated with immunotherapy with an antibody construct comprising a first domain which binds to a target antigen on the surface of a target cell, and a second domain which binds to human CD3 on the surface of a T cell according to embodiments (i) to (xii), (xiv) and (xv) of Aspect B), preferably in the immunotherapy of a neoplastic disease, wherein said combination product further comprises at least one IL-6 antagonist and/or IL-6R-antagonist, wherein said IL-6R-antagonist is tocilizumab.
(xvii) The method for the prevention or reduction of adverse events associated with immunotherapy with an antibody construct comprising a first domain which binds to a target antigen on the surface of a target cell, and a second domain which binds to human CD3 on the surface of a T cell according to embodiments (i) to (xvi) of Aspect B), preferably in the immunotherapy of a neoplastic disease, further comprising at least one corticosteroid, particularly dexamethasone and/or at least one IL-6 and/or IL-6R-antagonist, particularly tocilizumab, wherein said at least one corticosteroid and/or said IL-6 and/or IL-6R-antagonist is for administration to a patient in need thereof prior of, concomitant to, and/or after administration of said antibody construct (a).

### Aspect C)—Kits

(i) A kit comprising a combination product as defined in any of the preceding embodiments (i) to (xvi) of Aspect A) in a package, wherein the combination product is present in a single container, or the components of said combination product are present individually, said kit optionally further comprising at least one of a group comprising instructions for use, a device for administration of said combination product or a component thereof, at least one separately packed medium for reconstitution, a pharmaceutical carrier for at least one of said combination products or components thereof.

### Aspect D)—Products for Use

(i) An inhibitor/antagonist of TNF/TNFR that reduces TNF/TNFR signalling as defined in any of the foregoing embodiments (i) to (xvi) of Aspect A), wherein said inhibitor/antagonist of TNF/TNFR that reduces TNF/TNFR signalling is for use in the prevention of CRS in a patient suffering from a neoplastic disease.
(ii) The inhibitor/antagonist of TNF/TNFR that reduces TNF/TNFR signalling as defined in any of the foregoing embodiments (i) to (ii) for use in the prevention of CRS in a patient suffering from a neoplastic disease as defined in the preceding embodiment, wherein said patient is at risk of developing CRS.
(iii) The inhibitor/antagonist of TNF/TNFR that reduces TNF/TNFR signalling as defined in any of the foregoing embodiments (i) to (ii) for use in a method for the prevention of CRS in a patient suffering from a neoplastic disease who is at risk of developing CRS as defined in the preceding embodiments, wherein said patient is a person subjected to therapy with an antibody construct comprising a first domain which binds to a target antigen on the surface of a target cell, and a second domain which binds to human CD3 on the surface of a T cell as defined in any of the foregoing embodiments.

### Aspect E)—Second Medical Uses

(i) Use of an inhibitor/antagonist of TNF/TNFR that reduces TNF/TNFR signalling as defined in any of the foregoing embodiments (i) to (xvi) of Aspect A) wherein said inhibitor/antagonist of TNF/TNFR that reduces TNF/TNFR signalling is used in for the preparation of a medicament for the treatment or prevention of CRS in a patient suffering from a neoplastic disease.

### Aspect F)—Dosing

(i) An inhibitor/antagonist of TNF/TNFR that reduces TNF/TNFR signalling as defined in any of the foregoing embodiments (i) to (xvi) for use in the prevention of CRS in a patient suffering from a neoplastic disease and who is at risk of CRS as defined in the preceding embodiments, wherein said patient is a person subjected to therapy with an antibody construct comprising a first domain which binds to a target antigen on the surface of a target cell, and a second domain which binds to human CD3 on the surface of a T cell as defined in any of the foregoing embodiments, wherein a first dose of said inhibitor is administered before administration of a first dose of said antibody construct.

The term “antibody construct” refers to a molecule in which the structure and/or function is/are based on the structure and/or function of an antibody, e.g., of a full-length immunoglobulin molecule. Hence, an antibody construct immunospecifically binds to its target or antigen, and/or it comprises the heavy chain variable region (VH) and/or the light chain variable region (VL) of an antibody, or comprises domains derived therefrom. An antibody construct used according to the invention comprises the minimum structural requirements of an antibody which allow for immunospecific target binding. This minimum requirement may e.g. be defined by the presence of at least three light chain CDRs (i.e. CDR1, CDR2 and CDR3 of the VL region) and/or three heavy chain CDRs (i.e. CDR1, CDR2 and CDR3 of the VH region), preferably of all six CDRs. An antibody construct may hence be characterized by the presence of three or six CDRs in either one or both binding domains, and the skilled person knows where (in which order) those CDRs are located within the binding domain.

The definition of “antibody” according to the invention comprises full-length antibodies, also including camelid antibodies and other immunoglobulins generated by biotechnological or protein engineering methods or processes. These full-length antibodies may be for example monoclonal, recombinant, chimeric, deimmunized, humanized and human antibodies, as well as antibodies from other species such as mouse, hamster, rabbit, rat, goat, or non-human primates.

“Antibody constructs” used in accordance with the present invention may have the structure of a full-length immunoglobulin as it occurs naturally. For example, they may comprise (at least) two full-length antibody heavy chains and two full-length antibody light chains. However, given that the antibody constructs according used in accordance with the invention comprise one domain binding to a target antigen and another domain binding to CD3, they do not occur naturally, and they are markedly different in their function from naturally occurring products. An antibody construct used in accordance with the invention is hence an artificial “hybrid” molecule comprising at least two distinct binding domains with different specificities. It is emphasized that the antibody constructs disclosed herein may comprise more than two domains, e.g., they may comprise two identical or different target antigen binding domains and another domain binding to CD3. The target antigen binding domains may be identical, i.e. bind the same epitope, or they may bind different epitopes of the same of different target antigens.

“Antibody constructs” used in accordance with the present invention may also comprise fragments of full-length antibodies, such as VH, VHH, VL, (s)dAb, Fv, light chain (VL-CL), Fd (VH-CH1), heavy chain, Fab, Fab′, F(ab′)₂ or “r IgG” (“half antibody” consisting of a heavy chain and a light chain). Antibody constructs used in accordance with the invention may also comprise modified fragments of antibodies, also called antibody variants or antibody derivatives. Examples include, but are not limited to, scFv, di-scFv or bi(s)-scFv, scFv-Fc, scFv-zipper, scFab, Fab₂, Fab₃, diabodies, single chain diabodies, tandem diabodies (Tandab's), tandem di-scFv, tandem tri-scFv, “minibodies” exemplified by a structure which is as follows: (VH-VL-CH3)₂, (scFv-CH3)₂, ((scFv)₂-CH3+CH3), ((scFv)₂-CH3) or (scFv-CH3-scFv)₂, multibodies such as triabodies or tetrabodies, and single domain antibodies such as nanobodies or single variable domain antibodies comprising merely one variable region, which might be VHH, VH or VL, that specifically binds to an antigen or target independently of other variable regions or domains. Further possible formats of the antibody constructs used in accordance with the invention are cross bodies, maxi bodies, hetero Fc constructs, mono Fc constructs and scFc constructs. Examples for those formats will be described herein below.

Furthermore, the definition of the term “antibody construct” includes bivalent and polyvalent/multivalent constructs as well as bispecific and polyspecific/multispecific constructs, which specifically bind to two, three or more antigenic structures, through distinct binding domains. An antibody construct can have more binding valences than specificities, e.g. in a case where it has two binding domains for the first target and one binding domain for the second target (CD3), or vice versa, in which case the construct is trivalent and bispecific. In general, the term “bispecific” includes the meaning that an antibody construct binds to (at least) two different antigens, a target antigen and CD3.

Moreover, the definition of the term “antibody construct” includes molecules consisting of only one polypeptide chain as well as molecules consisting of two, three, four or more polypeptide chains, which chains can be either identical (homodimers, homotrimers or homo oligomers) or different (heterodimer, heterotrimer or heterooligomer). Examples for the above identified antibodies and their fragments, variants, derivatives and antibody constructs derived therefrom are described inter alia in Harlow and Lane, Antibodies: A laboratory manual, CSHL Press (1988); Kontermann and Dübel, Antibody Engineering, Springer, 2nd ed. 2010; and Little, Recombinant Antibodies for Immunotherapy, Cambridge University Press 2009.

The terms “binding domain” or “domain which binds to . . . ” or “domain” as far as it relates to the herein described “constructs” characterizes in connection with the present invention a domain of the antibody construct which immunospecifically binds to/interacts with/recognizes an epitope on the target or antigen. The structure and function of the first domain (binding to a target antigen), and preferably also the structure and/or function of the second domain (binding to CD3), is/are based on the structure and/or function of an antibody, e.g. of a full-length immunoglobulin molecule. The “binding domain” or “domain which binds to . . . ” may hence comprise the minimum structural requirements of an antibody which allow for immunospecific target binding. This minimum structural requirement of the first domain may e.g. be defined by the presence of at least three light chain CDRs (i.e. CDR1, CDR2 and CDR3 of the VL region) and/or of three heavy chain CDRs (i.e. CDR1, CDR2 and CDR3 of the VH region), preferably of all six CDRs. It is envisaged that the second domain also comprises this minimum structural requirement of an antibody which allow for the immunospecific target binding. More preferably, the second domain also comprises at least three light chain CDRs (i.e. CDR1, CDR2 and CDR3 of the VL region) and/or three heavy chain CDRs (i.e. CDR1, CDR2 and CDR3 of the VH region), preferably all six CDRs. A “domain which binds to” (or a “binding domain”) may typically comprise an antibody light chain variable region (VL) and an antibody heavy chain variable region (VH); however, it does not have to comprise both, but may comprise only one of VH or VL. Fd fragments, for example, often retain some antigen-binding function of the intact antigen-binding domain.

Examples for the format of a “domain which binds to” (or a “binding domain”) include, but are not limited to, full-length antibodies, fragments of full-length antibodies (such as VH, VHH, VL), (s)dAb, Fv, light chain (VL-CL), Fd (VH-CH1), heavy chain, Fab, Fab′, F(ab′)₂ or “r IgG” (“half antibody”)), antibody variants or derivatives such as scFv, di-scFv or bi(s)-scFv, scFv-Fc, scFv-zipper, scFab, Fab₂, Fab₃, diabodies, single chain diabodies, tandem diabodies (Tandab's), tandem di-scFv, tandem tri-scFv, “minibodies” (selected from formats such as (VH-VL-CH3)₂, (scFv-CH3)₂, ((scFv)₂-CH3+CH3)), ((scFv)₂-CH3) or (scFv-CH3-scFv)₂, multibodies such as triabodies or tetrabodies, and single domain antibodies such as nanobodies or single variable domain antibodies comprising merely one variable region, which might be VHH, VH or VL. Further examples for the format of a “domain which binds to” (or a “binding domain”) include (1) an antibody fragment or variant comprising VL, VH, CL and CH1 (such as Fab); (2) an antibody fragment or variant comprising two linked Fab fragments (such as a F(ab′)₂); (3) an antibody fragment or variant comprising VH and CH₁ (such as Fd); (4) an antibody fragment or variant comprising VL and CL (such as the light chain); (5) an antibody fragment or variant comprising VL and VH (such as Fv); (5) a dAb fragment (Ward et al., (1989) Nature 341:544-546), which has a VH domain; (6) an antibody variant comprising at least three isolated CDRs of the heavy and/or the light chain; and (7) a single chain Fv (scFv). Examples for embodiments of antibody constructs or binding domains used in accordance with the invention are e.g. described in WO 00/006605, WO 2005/040220, WO 2008/119567, WO 2010/037838, WO 2013/026837, WO 2013/026833, US 2014/0308285, US 2014/0302037, W 02014/144722, WO 2014/151910, and WO 2015/048272.

It is envisaged for the antibody construct used in accordance with the present invention that

a) the antibody construct is a single chain polypeptide or a single chain antibody construct,
b) the first domain is in the format of an scFv,
c) the second domain is in the format of an scFv,
d) the first and the second domain are connected via a linker, preferably a peptide linker, more preferably a glycine/serine linker, and/or
e) the antibody construct comprises at least one domain providing an extended serum half-life, such as an Fc-based domain, which may be located either at the C-terminal or N-terminal end of either the first or the second domain.

The antibody constructs used in accordance with the present invention are preferably “in vitro generated antibody constructs” and/or “recombinant antibody constructs”. In the context of the present invention, the term “in vitro generated” refers to an antibody construct according to the above definition where all or part of the binding domain or of a variable region (e.g., at least one CDR) is generated in a non-immune cell selection, e.g., in an in vitro phage display, on a protein chip or in any other method in which candidate amino acid sequences can be tested for their ability to bind to an antigen. This term thus preferably excludes sequences generated solely by genomic rearrangement in an immune cell in an animal. It is envisaged that the first and/or second domain of the antibody construct is produced by or obtainable by phage display or library screening methods rather than by grafting CDR sequences from a pre-existing (monoclonal) antibody into a scaffold. A “recombinant antibody construct” is an antibody construct generated or produced using (inter alia) recombinant DNA technology or genetic engineering.

The antibody constructs used in accordance with the present invention are envisaged to be monoclonal. As used herein, antibodies or antibody constructs that are denominated “monoclonal” (mAb) are obtained from a population of substantially homogeneous antibodies/antibody constructs, i.e., the individual antibodies/antibody constructs comprised in the population are identical (in particular with respect to their amino acid sequence) except for possible naturally occurring mutations and/or post-translational modifications (e.g., isomerizations, amidations) that may be present in minor amounts. Monoclonal antibodies/antibody constructs are highly specific, being directed against a single epitope within the antigen, in contrast to polyclonal antibody preparations which typically include different antibodies directed against different determinants (or epitopes). In addition to their specificity, monoclonal antibodies are advantageous in that they are synthesized by the hybridoma culture, hence uncontaminated by other immunoglobulins. The modifier “monoclonal” indicates the character of the antibody/antibody construct as being obtained from a substantially homogeneous population of antibodies and is not to be construed as requiring production of the antibody by any specific method.

For the preparation of monoclonal antibodies, any technique providing antibodies produced by continuous cell line cultures can be used. For example, monoclonal antibodies to be used may be made by the hybridoma method first described by Koehler et al., Nature, 256: 495 (1975), or may be made by recombinant DNA methods (see, e.g., U.S. Pat. No. 4,816,567). Examples for further techniques to produce human monoclonal antibodies include the trioma technique, the human B-cell hybridoma technique (Kozbor, Immunology Today 4 (1983), 72) and the EBV-hybridoma technique (Cole et al., Monoclonal Antibodies and Cancer Therapy, Alan R. Liss, Inc. (1985), 77-96).

Hybridomas can then be screened using standard methods, such as enzyme-linked immunosorbent assay (ELISA) and surface plasmon resonance (BIACORE™) analysis, to identify one or more hybridomas that produce an antibody that immunospecifically binds to a specified antigen. Any form of the relevant antigen may be used as the immunogen, e.g., recombinant antigen, naturally occurring forms, any variants or fragments thereof, as well as an antigenic peptide thereof. Surface plasmon resonance as employed in the BIAcore™ system can be used to increase the efficiency of phage antibodies/antibody constructs which bind to an epitope of a target antigen (Schier, Human Antibodies Hybridomas 7 (1996), 97-105; Malmborg, J. Immunol. Methods 183 (1995), 7-13).

Another exemplary method of making antibody constructs or binding domains includes screening protein expression libraries, e.g., phage display or ribosome display libraries. Phage display is described, for example, in Ladner et al., U.S. Pat. No. 5,223,409; Smith (1985) Science 228:1315-1317, Clackson et al., Nature, 352: 624-628 (1991) and Marks et al., J. Mol. Biol., 222: 581-597 (1991).

In addition to the use of display libraries, the relevant antigen can be used to immunize a non-human animal, e.g., a rodent (such as a mouse, hamster, rabbit or rat). In one embodiment, the non-human animal includes at least a part of a human immunoglobulin gene. For example, it is possible to engineer mouse strains deficient in mouse antibody production with large fragments of the human Ig (immunoglobulin) loci. Using the hybridoma technology, antigen-specific monoclonal antibodies derived from the genes with the desired specificity may be produced and selected. See, e.g., Xenomouse™, Green et al. (1994) Nature Genetics 7:13-21, US 2003-0070185, WO 96/34096, and WO 96/33735.

A monoclonal antibody can also be obtained from a non-human animal, and then modified, e.g., humanized, deimmunized, rendered chimeric etc., using recombinant DNA techniques known in the art. Examples of modified antibody constructs or binding domains include humanized variants of non-human antibodies/antibody constructs, “affinity matured” antibody constructs or binding domains (see, e.g. Hawkins et al. J. Mol. Biol. 254, 889-896 (1992) and Lowman et al., Biochemistry 30, 10832-10837 (1991)) and antibody variants or mutants with altered effector function(s) (see, e.g., U.S. Pat. No. 5,648,260, Kontermann and Dübel (2010), loc. cit. and Little (2009), loc. cit.).

In immunology, affinity maturation is the process by which B cells produce antibodies with increased affinity for antigen during the course of an immune response. With repeated exposures to the same antigen, a host will produce antibodies of successively greater affinities. Like the natural prototype, the in vitro affinity maturation is based on the principles of mutation and selection. The in vitro affinity maturation has successfully been used to optimize antibodies, antibody fragments, antibody variants, antibody constructs or binding domains. Random mutations inside the CDRs are introduced using radiation, chemical mutagens or error-prone PCR. In addition, the genetic diversity can be increased by chain shuffling. Two or three rounds of mutation and selection using display methods like phage display usually results in antibodies, antibody fragments, antibody variants, antibody constructs or binding domains with affinities in the low nanomolar range.

A preferred type of an amino acid substitutional variation of the antibody constructs or binding domains used in accordance with the invention involves substituting one or more residues within the hypervariable region of a parent antibody structure (e.g. a humanized or human antibody structure). Generally, the resulting variant(s) selected for further development will have improved biological properties relative to the parent antibody structure from which they are generated. A convenient way for generating such substitutional variants involves affinity maturation using phage display. Briefly, several sites of the hypervariable region (e. g. 6-7 sites) are mutated to generate all possible amino acid substitutions at each site. The variants thus generated are displayed in a monovalent fashion from filamentous phage particles as fusions to the gene III product of M13 packaged within each particle. The phage-displayed variants are then screened for their biological activity (e.g. binding affinity) as disclosed herein. To identify candidate hypervariable region sites contributing significantly to antigen binding (candidates for modification), alanine scanning mutagenesis can also be performed. Alternatively, or additionally, it may be beneficial to analyze a crystal structure of the complex between the antigen and the antibody construct or the binding domain to identify contact points between the binding domain and its specific antigen. Such contact residues and neighbouring residues are candidates for substitution according to the techniques elaborated herein. Once such variants are generated, the panel of variants is subjected to screening as described herein and antibodies, their antigen-binding fragments, antibody constructs or binding domains with superior properties in one or more relevant assays may be selected for further development.

The antibody constructs and binding domains used in accordance with the present invention specifically include “chimeric” versions in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments or variants of such antibodies, so long as they exhibit the desired biological activity (U.S. Pat. No. 4,816,567; Morrison et al., Proc. Natl. Acad. Sci. USA, 81: 6851-6855 (1984)). Chimeric antibody constructs or binding domains of interest herein include “primitized” antibody constructs comprising variable domain antigen-binding sequences derived from a non-human primate (e.g., Old World Monkey, Ape etc.) and human constant region sequences. A variety of approaches for making chimeric antibodies or antibody constructs have been described. See e.g., Morrison et al., Proc. Natl. Acad. Sci. U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., EP 0171496; EP 0173494; and GB 2177096.

An antibody, antibody construct, antibody fragment, antibody variant or binding domain may also be modified by specific deletion of human T cell epitopes (a method called “deimmunization”) using methods disclosed for example in WO 98/52976 or WO 00/34317. Briefly, the heavy and light chain variable regions of an antibody, antibody construct or binding domain can be analyzed for peptides that bind to MHC class II; these peptides represent potential T cell epitopes (as defined e.g. in WO 98/52976 and WO 00/34317). For detection of potential T cell epitopes, a computer modeling approach termed “peptide threading” can be applied, and in addition a database of human MHC class II binding peptides can be searched for motifs present in the VH and VL sequences, as described in WO 98/52976 and WO 00/34317. These motifs bind to any of the 18 major MHC class II DR allotypes, and thus constitute potential T cell epitopes. Potential T cell epitopes detected can be eliminated by substituting small numbers of amino acid residues in the variable domains or variable regions, or preferably, by single amino acid substitutions. Typically, conservative substitutions are made. Often, but not exclusively, an amino acid common to a position in human germline antibody sequences may be used. Human germline sequences are disclosed e.g. in Tomlinson, et al. (1992) J. Mol. Biol. 227:776-798; Cook, G. P. et al. (1995) Immunol. Today Vol. 16 (5): 237-242; and Tomlinson et al. (1995) EMBO J. 14: 14:4628-4638. The V BASE directory (www2.mrc-lmb.cam.ac.uk/vbase/list2.php) provides a comprehensive directory of human immunoglobulin variable region sequences (compiled by Tomlinson, L A. et al. MRC Centre for Protein Engineering, Cambridge, UK). These sequences can be used as a source of human sequence, e.g., for framework regions and CDRs. Consensus human framework regions can also be used, for example as described in U.S. Pat. No. 6,300,064.

“Humanized” antibodies, variants or fragments thereof, antibody constructs and binding domains are based on immunoglobulins of mostly human sequences, which contain (a) minimal sequence(s) derived from non-human immunoglobulin. For the most part, humanized antibodies, variants or fragments thereof, antibody constructs and binding domains are based on human immunoglobulins (recipient antibodies) in which residues from a hypervariable region or CDR are replaced by residues from a hypervariable region or CDR of a non-human species (donor antibody) such as a rodent (e.g. mouse, hamster, rat or rabbit) having the desired specificity, affinity, capacity and/or biological activity. In some instances, Fv framework region (FR) residues of the human immunoglobulin are replaced by corresponding non-human residues. Furthermore, “humanized” antibodies, variants or fragments thereof, antibody constructs and binding domains as used herein may also comprise residues which are found neither in the recipient antibody nor the donor antibody. These modifications are made to further refine and optimize antibody performance. The humanized antibodies, variants or fragments thereof, antibody constructs and binding domains may also comprise at least a portion of an immunoglobulin constant region (such as Fc), typically that of a human immunoglobulin. For further details, see Jones et al., Nature, 321: 522-525 (1986); Reichmann et al., Nature, 332: 323-329 (1988); and Presta, Curr. Op. Struct. Biol., 2: 593-596 (1992).

Humanized antibodies, variants or fragments thereof, antibody constructs and binding domains can be generated by replacing sequences of the (Fv) variable region that are not directly involved in antigen binding with equivalent sequences from human (Fv) variable regions. Exemplary methods for generating such molecules are provided by Morrison (1985) Science 229:1202-1207; by Oi et al. (1986) BioTechniques 4:214; and by U.S. Pat. Nos. 5,585,089; 5,693,761; 5,693,762; 5,859,205; and 6,407,213. These methods include isolating, manipulating, and expressing the nucleic acid sequences that encode all or part of immunoglobulin (Fv) variable regions from at least one of a heavy or light chain. Such nucleic acids may be obtained from a hybridoma producing an antibody against a predetermined target, as described above, as well as from other sources. The recombinant DNA encoding the humanized antibody, variant or fragment thereof, antibody construct or binding domain can then be cloned into an appropriate expression vector.

Humanized antibodies, variants or fragments thereof, antibody constructs and binding domains may also be produced using transgenic animals such as mice that express human heavy and light chain genes but are incapable of expressing the endogenous mouse immunoglobulin heavy and light chain genes. Winter describes an exemplary CDR grafting method that may be used to prepare the humanized molecules described herein (U.S. Pat. No. 5,225,539). All the CDRs of a given human sequence may be replaced with at least a portion of a non-human CDR, or only some of the CDRs may be replaced with non-human CDRs. It is only necessary to replace the number of CDRs required for binding of the humanized molecule to a predetermined antigen.

A humanized antibody, variant or fragment thereof, antibody construct or binding domain can be optimized by the introduction of conservative substitutions, consensus sequence substitutions, germline substitutions and/or back mutations. Such altered immunoglobulin molecules can be made by any of several techniques known in the art, (e.g., Teng et al., Proc. Natl. Acad. Sci. U.S.A., 80: 7308-7312, 1983; Kozbor et al., Immunology Today, 4: 7279, 1983; Olsson et al., Meth. Enzymol., 92: 3-16, 1982, and EP 239 400).

Human anti-mouse antibody (HAMA) responses have led the industry to prepare chimeric or otherwise humanized antibodies/antibody constructs. It is however expected that certain human anti-chimeric antibody (HACA) responses will be observed, particularly in chronic or multi-dose utilizations of an antibody or antibody construct. Thus, it would be desirable to provide antibody constructs comprising a human binding domain against a target antigen and/or a human binding domain against CD3, in order to vitiate concerns and/or effects of HAMA or HACA response.

Therefore, according to one embodiment, the antibody construct, the first binding domain and/or the second binding domain are “human”. The term “human antibody”, “human antibody construct” and “human binding domain” includes antibodies, antibody constructs and binding domains, respectively, having antibody-derived regions such as variable and constant regions or domains which correspond substantially to human germline immunoglobulin sequences known in the art, including, for example, those described by Kabat et al. (1991) (loc. cit.). The human antibody constructs or binding domains used in accordance with the invention may include amino acid residues not encoded by human germline immunoglobulin sequences (e.g., mutations introduced by random or site-specific mutagenesis in vitro or by somatic mutation in vivo), for example in the CDRs, and particularly in CDR3. The human antibody constructs or binding domains can have at least one, two, three, four, five, or more positions replaced with an amino acid residue that is not encoded by the human germline immunoglobulin sequence. The definition of human antibodies, antibody constructs and binding domains as used herein also contemplates fully human antibodies, antibody constructs and binding domains which include only non-artificially and/or genetically altered human sequences of antibodies as those can be derived by using technologies or systems such as the Xenomouse.

Antibody constructs comprising at least one human binding domain avoid some of the problems associated with antibodies or antibody constructs that possess non-human such as rodent (e.g. murine, rat, hamster or rabbit) variable and/or constant regions. The presence of such rodent derived proteins can lead to the rapid clearance of the antibodies or antibody constructs or can lead to the generation of an immune response against the antibody or antibody construct by a patient. To avoid the use of rodent-derived antibody constructs, humanized or fully human antibody constructs can be generated through the introduction of human antibody functions into a rodent so that the rodent produces fully human antibodies.

The ability to clone and reconstruct megabase-sized human loci in YACs and to introduce them into the mouse germline provides a powerful approach to elucidating the functional components of very large or crudely mapped loci as well as generating useful models of human disease. Furthermore, the use of such technology for substitution of mouse loci with their human equivalents could provide unique insights into the expression and regulation of human gene products during development, their communication with other systems, and their involvement in disease induction and progression.

An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies as well as their role in B-cell development. Furthermore, such a strategy could provide an ideal source for production of fully human monoclonal antibodies (mAbs)—an important milestone towards fulfilling the promise of antibody therapy in human disease. Fully human antibodies or antibody constructs derived therefrom are expected to minimize the immunogenic and allergic responses intrinsic to mouse or mouse-derivatized mAbs and thus to increase the efficacy and safety of the administered antibodies/antibody constructs. The use of fully human antibodies or antibody constructs can be expected to provide a substantial advantage in the treatment of chronic and recurring human diseases, such as inflammation, autoimmunity, and cancer, which require repeated compound administrations.

One approach towards this goal was to engineer mouse strains deficient in mouse antibody production with large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human mAbs with the desired specificity could be readily produced and selected. This general strategy was demonstrated in connection with the generation of the first XenoMouse mouse strains (see Green et al. Nature Genetics 7:13-21 (1994)). The XenoMouse strains were engineered with yeast artificial chromosomes (YACs) containing 245 kb and 190 kb-sized germline configuration fragments of the human heavy chain locus and kappa light chain locus, respectively, which contained core variable and constant region sequences. The human Ig containing YACs proved to be compatible with the mouse system for both rearrangement and expression of antibodies and were capable of substituting for the inactivated mouse Ig genes. This was demonstrated by their ability to induce B cell development, to produce an adult-like human repertoire of fully human antibodies, and to generate antigen-specific human mAbs. These results also suggested that introduction of larger portions of the human Ig loci containing greater numbers of V genes, additional regulatory elements, and human Ig constant regions might recapitulate substantially the full repertoire that is characteristic of the human humoral response to infection and immunization. The work of Green et al. was extended to the introduction of greater than approximately 80% of the human antibody repertoire through introduction of megabase sized, germline configuration YAC fragments of the human heavy chain loci and kappa light chain loci, respectively. See Mendez et al. Nature Genetics 15:146-156 (1997) and U.S. patent application Ser. No. 08/759,620.

The production of the XenoMouse model is further discussed and delineated in U.S. patent application Ser. No. 07/466,008, Ser. No. 07/610,515, Ser. No. 07/919,297, Ser. No. 07/922,649, Ser. No. 08/031,801, Ser. No. 08/112,848, Ser. No. 08/234,145, Ser. No. 08/376,279, Ser. No. 08/430,938, Ser. No. 08/464,584, Ser. No. 08/464,582, Ser. No. 08/463,191, Ser. No. 08/462,837, Ser. No. 08/486,853, Ser. No. 08/486,857, Ser. No. 08/486,859, Ser. No. 08/462,513, Ser. No. 08/724,752, and Ser. No. 08/759,620; and U.S. Pat. Nos. 6,162,963; 6,150,584; 6,114,598; 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits, J. Exp. Med. 188:483-495 (1998), EP 0 463 151 B1, WO 94/02602, WO 96/34096, WO 98/24893, WO 00/76310, and WO 03/47336.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more VH genes, one or more DH genes, one or more JH genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806; 5,625,825; 5,625,126; 5,633,425; 5,661,016; 5,770,429; 5,789,650; 5,814,318; 5,877,397; 5,874,299; and 6,255,458 each to Lonberg and Kay, U.S. Pat. Nos. 5,591,669 and 6,023,010 to Krimpenfort and Berns, U.S. Pat. Nos. 5,612,205; 5,721,367; and U.S. Pat. No. 5,789,215 to Berns et al., and U.S. Pat. No. 5,643,763 to Choi and Dunn, and GenPharm International U.S. patent application Ser. No. 07/574,748, Ser. No. 07/575,962, Ser. No. 07/810,279, Ser. No. 07/853,408, Ser. No. 07/904,068, Ser. No. 07/990,860, Ser. No. 08/053,131, Ser. No. 08/096,762, Ser. No. 08/155,301, Ser. No. 08/161,739, Ser. No. 08/165,699, Ser. No. 08/209,741. See also EP 0 546 073 B1, WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175. See further Taylor et al. (1992), Chen et al. (1993), Tuaillon et al. (1993), Choi et al. (1993), Lonberg et al. (1994), Taylor et al. (1994), and Tuaillon et al. (1995), Fishwild et al. (1996).

Kirin has also demonstrated the generation of human antibodies from mice in which, through microcell fusion, large pieces of chromosomes, or entire chromosomes, have been introduced. See European Patent Application Nos. 773 288 and 843 961. Xenerex Biosciences is developing a technology for the potential generation of human antibodies. In this technology, SCID mice are reconstituted with human lymphatic cells, e.g., B and/or T cells. Mice are then immunized with an antigen and can generate an immune response against the antigen. See U.S. Pat. Nos. 5,476,996; 5,698,767; and 5,958,765.

In some embodiments, the antibody constructs used in accordance with the invention are “isolated” or “substantially pure” antibody constructs. “Isolated” or “substantially pure”, when used to describe the antibody constructs disclosed herein, means an antibody construct that has been identified, separated and/or recovered from a component of its production environment. Preferably, the antibody construct is free or substantially free of association with all other components from its production environment. Contaminant components of its production environment, such as that resulting from recombinant transfected cells, are materials that could interfere with diagnostic or therapeutic uses for the antibody construct, and may include enzymes, hormones, and other proteinaceous or non-proteinaceous compounds. It is understood that the isolated or substantially pure antibody construct may constitute from 5% to 99.9% by weight of the total protein/polypeptide content in a given sample, depending on the circumstances. The desired antibody construct may be produced at a significantly higher concentration using an inducible promoter or high expression promoter. The definition includes the production of an antibody construct in a wide variety of organisms and/or host cells that are known in the art. In certain embodiments, the antibody construct will be purified (1) to a degree enough to obtain at least 15 residues of N-terminal or internal amino acid sequence by use of a spinning cup sequenator, or (2) to homogeneity by SDS-PAGE under non-reducing or reducing conditions using Coomassie Blue or, preferably, silver staining. Usually, however, an isolated antibody construct will be prepared by at least one purification step.

According to one embodiment, the entire antibody construct and/or the binding domains are in the form of one or more polypeptides or in the form of proteins. In addition to proteinaceous parts, such polypeptides or proteins may include non-proteinaceous parts (e.g. chemical linkers or chemical cross-linking agents such as glutaraldehyde).

Peptides are short chains of amino acid monomers linked by covalent peptide (amide) bonds. Hence, peptides fall under the broad chemical classes of biological oligomers and polymers Amino acids that are part of a peptide or polypeptide chain are termed “residues” and can be consecutively numbered. All peptides except cyclic peptides have an N-terminal residue at one end and a C-terminal residue at the other end of the peptide. An oligopeptide consists of only a few amino acids (usually between two and twenty). A polypeptide is a longer, continuous, and unbranched peptide chain. Peptides are distinguished from proteins on the basis of size, and as an arbitrary benchmark can be understood to contain approximately 50 or fewer amino acids. Proteins consist of one or more polypeptides, usually arranged in a biologically functional way. While aspects of the lab techniques applied to peptides versus polypeptides and proteins differ (e.g., the specifics of electrophoresis, chromatography, etc.), the size boundaries that distinguish peptides from polypeptides and proteins are not absolute. Therefore, in the context of the present invention, the terms “peptide”, “polypeptide” and “protein” may be used interchangeably, and the term “polypeptide” is often preferred.

Polypeptides may further form multimers such as dimers, trimers and higher oligomers, which consist of more than one polypeptide molecule. Polypeptide molecules forming such dimers, trimers etc. may be identical or non-identical. The corresponding structures of higher order of such multimers are, consequently, termed homo- or heterodimers, homo- or heterotrimers etc. An example for a hereteromultimer is an antibody or immunoglobulin molecule, which, in its naturally occurring form, consists of two identical light polypeptide chains and two identical heavy polypeptide chains. The terms “peptide”, “polypeptide” and “protein” also refer to naturally modified peptides/polypeptides/proteins wherein the modification is accomplished e.g. by post-translational modifications like glycosylation, acetylation, phosphorylation and the like. A “peptide”, “polypeptide” or “protein” when referred to herein may also be chemically modified such as pegylated. Such modifications are well known in the art and described herein below.

The terms “(specifically or immunospecifically) binds to”, “(specifically or immunospecifically) recognizes”, or “(specifically or immunospecifically) reacts with” mean in accordance with this invention that an antibody construct or a binding domain interacts or (immuno-)specifically interacts with a given epitope on the target molecule (antigen) and CD3, respectively. This interaction or association occurs more frequently, more rapidly, with greater duration, with greater affinity, or with some combination of these parameters, to an epitope on the specific target than to alternative substances (non-target molecules). Because of the sequence similarity between homologous proteins in different species, an antibody construct or a binding domain that immunospecifically binds to its target (such as a human target) may, however, cross-react with homologous target molecules from different species (such as, from non-human primates). The term “specific/immunospecific binding” can hence include the binding of an antibody construct or binding domain to epitopes or structurally related epitopes in more than one species.

In the context of the present invention, the term “epitope” refers to the part or region of the antigen that is recognized/immunospecifically recognized by the binding domain. An “epitope” is antigenic, and thus the term epitope is sometimes also referred to as “antigenic structure” or “antigenic determinant”. The part of the binding domain that binds to the epitope is called a paratope. Specific binding is believed to be accomplished by specific motifs in the amino acid sequence of the binding domain and the antigen. Thus, binding is achieved because of their primary, secondary and/or tertiary structure as well as the result of potential secondary modifications of said structures. The specific interaction of the paratope with its antigenic determinant may result in a simple binding of said site to the antigen. In some cases, the specific interaction may alternatively or additionally result in the initiation of a signal, e.g. due to the induction of a change of the conformation of the antigen, an oligomerization of the antigen, etc.

The epitopes of protein antigens are divided into two categories, conformational epitopes and linear epitopes, based on their structure and interaction with the paratope. A conformational epitope is composed of discontinuous sections of the antigen's amino acid sequence. These epitopes interact with the paratope based on the three-dimensional surface features and shape or tertiary structure (folding) of the antigen. Methods of determining the conformation of epitopes include, but are not limited to, x-ray crystallography, two-dimensional nuclear magnetic resonance (2D-NMR) spectroscopy and site-directed spin labelling and electron paramagnetic resonance (EPR) spectroscopy. By contrast, linear epitopes interact with the paratope based on their primary structure. A linear epitope is formed by a continuous sequence of amino acids from the antigen and typically includes at least 3 or at least 4, and more usually, at least 5 or at least 6 or at least 7, for example, about 8 to about 10 amino acids in a unique sequence.

A method for epitope mapping is described in the following: A pre-defined region (a contiguous amino acid stretch) within the target protein is exchanged/replaced, preferably with a corresponding region, of a different or even a similar (e.g., a homologous) non-target antigen peptide fragment provided that the binding domain is not cross-reactive with the non-target antigen peptide fragment. These chimeras are expressed on the surface of host cells (such as CHO cells). Binding of the antibody or antibody construct can be tested via FACS analysis. When the binding of the antibody or antibody construct to the chimeric molecule is entirely abolished, or when a significant binding decrease is observed, it can be concluded that the region of the target antigen which was removed from this chimeric molecule is relevant for the immunospecific epitope-paratope recognition. Said decrease in binding is preferably at least 10%, 20%, 30%, 40%, or 50%; more preferably at least 60%, 70%, or 80%, and most preferably 90%, 95% or even 100% in comparison to the binding to the non-modified (wild-type) target antigen, which is set to be 100%. Alternatively, the above described epitope mapping analysis can be modified by introducing one or more point mutations into the sequence of the target antigen. These point mutations can e.g. reflect the differences between the target antigen and a similar (e.g., a homologous) polypeptide.

A further method to determine the contribution of a specific residue of a target antigen to the recognition by an antibody construct or binding domain is alanine scanning (see e.g. Morrison K L & Weiss G A. Curr Opin Chem Biol. 2001 June; 5(3):302-7), where each residue to be analyzed is replaced by alanine, e.g. via site-directed mutagenesis. Alanine is used because of its non-bulky, chemically inert, methyl functional group that nevertheless mimics the secondary structure references that many of the other amino acids possess. Sometimes bulky amino acids such as valine or leucine can be used in cases where conservation of the size of mutated residues is desired.

The interaction between the binding domain and the epitope of the target antigen implies that a binding domain exhibits appreciable or significant affinity for the epitope/the target antigen and, generally, does not exhibit significant affinity for proteins or antigens other than the target antigen—notwithstanding the above discussed cross-reactivity with homologous targets e.g. from other species. “Significant affinity” includes binding with an affinity (dissociation constant, KD) of ≤10⁻⁶ M. Preferably, binding is considered specific when the binding affinity is ≤10⁻⁷ M, ≤10⁻⁸ M, ≤10⁻⁹ M, ≤10¹⁰ M, or even ≤10⁻¹¹ M, or ≤10⁻¹² M. Whether a binding domain (immuno-)specifically reacts with or binds to a target can be tested readily e.g. by comparing the affinity of said binding domain to its desired target protein or antigen with the affinity of said binding domain to non-target proteins or antigens. Preferably, an antibody construct used in accordance with the invention does not significantly bind to proteins or antigens other than target antigen(s) or CD3, respectively—unless any further binding domain(s) directed against a further target is/are deliberately introduced into the antibody construct used in accordance with the invention, in which case the binding of that binding domain to its specific target is also provided by the present invention.

It is envisaged that the affinity of the first domain for the target antigen(s) is ≤100 nM, ≤90 nM, ≤80 nM, ≤70 nM, ≤60 nM, ≤50 nM, ≤40 nM, ≤30 nM, or ≤20 nM. These values are preferably measured in a cell-based assay, such as a Scatchard assay. See Example 4. Other methods of determining the affinity are also well-known. It is furthermore envisaged that the affinity of the second domain for CD3 (e.g. human CD3) is ≤100 nM, ≤90 nM, ≤80 nM, ≤70 nM, ≤60 nM, ≤50 nM, ≤40 nM, ≤30 nM, ≤20 nM, or ≤10 nM. These values are preferably measured in a surface plasmon resonance assay, such as a Biacore assay.

The term “does not significantly bind” means that an antibody construct or binding domain used in accordance with the present invention does not bind to a protein or antigen other than the target antigen(s) or CD3, when said protein or antigen is expressed on the surface of a cell. The antibody construct hence shows reactivity of ≤30%, preferably ≤20%, more preferably ≤10%, particularly preferably ≤9%, ≤8%, ≤7%, ≤6%, ≤5%, ≤4%, ≤3%, ≤2%, or ≤1% with proteins or antigens other than the target antigen(s) or CD3 (when said proteins or antigens are expressed on the surface of a cell), whereby binding to the target antigen(s) or CD3, respectively, is set to be 100%. The “reactivity” can e.g. be expressed in an affinity value (see above).

It is envisaged that the antibody construct used in accordance with the invention (and more specifically its first domain) does not bind or does not significantly bind to the target antigen(s) homologues, more specifically to human the target antigen(s) homologues and/or to macaque/cyno the target antigen(s) homologues. It is also envisaged that the antibody construct does not bind or does not significantly bind to (human or macaque/cyno) the target antigen(s) homologues on the surface of a target cell. It is hence envisaged that the first domain of the antibody construct used in accordance with the invention does not bind or does not significantly bind to similar, but different (e.g., homologous) antigen(s), preferably on the surface of a target cell.

The first domain of the antibody construct used in accordance with the invention binds to the target antigen(s) on the surface of a target cell. The “target cell” can be any prokaryotic or eukaryotic cell expressing the target antigen(s) on its surface; preferably the target cell is a cell that is part of the human or animal body, such as a specific the target antigen(s)-expressing cancer or tumor cell or a cell of target antigen-positive neoplasm. The first domain may hence bind to the target antigen(s) expressed by naturally expressing cells or cell lines (such as human cancer lines), and/or by cells or cell lines transformed or (stably/transiently) transfected with nucleic acids encoding the target antigen(s). In one embodiment, the first domain binds to the target antigen(s) is used as a target molecule in a cell-based binding assay such as Scatchard. It is furthermore envisaged that the antibody construct/its first domain binds to human the target antigen(s) on the surface of a target cell. In this connection, the term “target antigen” relates to a molecular structure, e.g. a protein or a part thereof that is specifically recognized on a target cell. The target may be a tumor antigen, a neo antigen, an antigen that is usually not found on a differentiated cell, e.g. a protein that is a characteristic marker for a cancer cell, a neoplastic cell, or a cell that expresses an antigen that is foreign to the host body, e.g. a viral or bacterial antigen, for example an oncoviral antigen that is associated with an infection of a cell by an oncovirus, which is the causative agent for the proliferation of an infected cell by cell division, wherein the cell would normally, under non-infected conditions, no longer or not at all, proliferate, e.g., cells infected by oncogenic viruses, such as human papillomaviruses, hepatitis viruses, etc., that proliferate and give rise to neoplastic tissues.

Whether or not an antibody, antibody construct or binding domain binds to the same epitope of the target antigen(s) on the surface of a target cell as another given antibody, antibody construct or binding domain can be measured by different analyses as described herein, e.g. by epitope mapping with chimeric or mutated target antigen molecules, as described herein above. Other methods of determining epitopes are described herein, such as alanine scanning

Whether or not an antibody or antibody construct competes for binding to an antigen on the surface of a target cell with another given antibody or antibody construct can be measured in a competition assay such as a competitive ELISA. Avidin-coupled microparticles (beads) can also be used. Similarly, to an avidin-coated ELISA plate, when reacted with a biotinylated protein, each of these beads can be used as a substrate on which an assay can be performed. Antigen is coated onto a bead and then precoated with the first antibody. The second antibody is added, and any additional binding is determined. Read-out occurs via flow cytometry. Preferably a cell-based competition assay is used, using either cells that naturally express the target antigen(s) or cells that were stably or transiently transformed with nucleic acids encoding the target antigen(s). The term “competes for binding”, in the present context, means that competition occurs between the two tested antibodies of at least 20%, at least 30%, at least 40%, at least 50%, at least 60%, at least 70%, at least 80% or at least 90%, as determined by any one of the assays disclosed above, preferably the cell-based assay. The same analysis can of course be applied for other targets such as CD3.

Competitive antibody binding assays include assays determining the competitive binding of two antibodies/antibody constructs to a cell surface bound antigen. Common methods aim to detect binding of two antibodies/antibody constructs, A and B, to the same antigen on the surface of a cell may include steps of:
a) blocking of the cell surface antigen by pre-incubation of cells with antibody/antibody construct A followed by a sub-maximal addition of labeled antibody/antibody construct B and detecting the binding of B compared with binding in the absence of A;
b) titration (i.e. adding different amounts) of antibody/antibody construct A in the presence of sub-maximal amounts of labeled antibody/antibody construct B and detecting the effect on binding of B; or
c) co-titration of A and B, wherein both antibodies/antibody constructs are incubated together at maximal concentration and detecting whether the total binding equals or exceeds that of either A or B alone, i.e. a method which cannot be affected by the order of addition or relative amounts of the antibodies/antibody constructs.

When two antibodies/antibody constructs A and B compete for a cell surface bound antigen, the antibodies will very often compete in blocking assays independently from the order of the addition of the antibodies. In other words, competition is detected if the assay is carried out in either direction. However, this is not always the case, and under certain circumstances the order of the addition of the antibodies or the direction of the assay may influence the signal generated. This may be due to differences in affinities or avidities of the potentially competing antibodies/antibody constructs. If the order of the addition has a significant effect on the signal generated, it is concluded that the two antibodies/antibody constructs do compete if competition is detected in at least one order.

In the context of the present invention, the term “variable” refers to those portions of antibody or immunoglobulin domains that exhibit variability in their sequence and that are involved in determining the specificity and binding affinity of a particular antibody (i.e., the “variable region(s)”). Usually, the pairing of a heavy chain variable region (VH) and a light chain variable region (VL) together forms a single antigen-binding site.

Variability is not evenly distributed throughout the variable regions of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable regions are called the “framework” (FR) regions and provide a scaffold for the six CDRs in three-dimensional space to form an antigen-binding surface. The variable regions of naturally occurring antibody heavy and light chains each comprise four FR regions (FR1, FR2, FR3, and FR4), largely adopting a β-sheet configuration. Together with the CDRs, they form the following sequence within a variable heavy or light chain: FR1-CDR1-FR2-CDR2-FR3-CDR3-FR4. The hypervariable regions in each chain are held together in proximity by the framework regions and, usually together with the hypervariable regions from the other chain, contribute to the formation of the antigen-binding site (see Kabat et al., loc. cit.).

The terms “CDR”, and its plural “CDRs”, refer to the complementarity determining region of which three make up the binding character of a light chain variable region (CDR-L1, CDR-L2 and CDR-L3) and three make up the binding character of a heavy chain variable region (CDR-H1, CDR-H2 and CDR-H3). CDRs contain most of the residues responsible for specific interactions of the antibody (or antibody construct or binding domain) with the antigen and hence contribute to the functional activity of an antibody molecule: they are the main determinants of antigen specificity.

The exact definition of CDR boundaries and lengths is subject to different classification and numbering systems. CDRs may therefore be referred to by Kabat, Chothia, contact or any other boundary definitions, including the numbering system described herein. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat (an approach based on cross-species sequence variability), Chothia (an approach based on crystallographic studies of antigen-antibody complexes), and/or MacCallum (Kabat et al., loc. cit.; Chothia et al., J. Mol. Biol, 1987, 196: 901-917; and MacCallum et al., J. Mol. Biol, 1996, 262: 732). Still another standard for characterizing the antigen binding site is the AbM definition used by Oxford Molecular's AbM antibody modeling software. See, e.g., Protein Sequence and Structure Analysis of Antibody Variable Domains. In: Antibody Engineering Lab Manual (Ed.: Duebel, S. and Kontermann, R., Springer-Verlag, Heidelberg). To the extent that two residue identification techniques define regions of overlapping, but not identical regions, they can be combined to define a hybrid CDR. However, the numbering in accordance with the so-called Kabat system is preferred.

Typically, CDRs form a loop structure that can be classified as a canonical structure. The term “canonical structure” refers to the main chain conformation that is adopted by the antigen binding (CDR) loops. From comparative structural studies, it has been found that five of the six antigen binding loops have only a limited repertoire of available conformations. Each canonical structure can be characterized by the torsion angles of the polypeptide backbone. Corresponding loops between antibodies may, therefore, have very similar three-dimensional structures, despite high amino acid sequence variability in most parts of the loops (Chothia and Lesk, J. Mol. Biol., 1987, 196: 901; Chothia et al., Nature, 1989, 342: 877; Martin and Thornton, J. Mol. Biol, 1996, 263: 800). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class can therefore be made based on the presence of these key amino acid residues.

The term “canonical structure” may also include considerations as to the linear sequence of the antibody, for example, as catalogued by Kabat (Kabat et al., loc. cit.). The Kabat numbering scheme (system) is a widely adopted standard for numbering the amino acid residues of an antibody variable region in a consistent manner and is the preferred scheme applied in the present invention as also mentioned elsewhere herein. Additional structural considerations can also be used to determine the canonical structure of an antibody. For example, those differences not fully reflected by Kabat numbering can be described by the numbering system of Chothia et al. and/or revealed by other techniques, for example, crystallography and two- or three-dimensional computational modeling. Accordingly, a given antibody sequence may be placed into a canonical class which allows for, among other things, identifying appropriate class sequences (e.g., based on a desire to include a variety of canonical structures in a library). Kabat numbering of antibody amino acid sequences and structural considerations as described by Chothia et al., loc. cit. and their implications for construing canonical aspects of antibody structure, are described in the literature. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988.

The CDR3 of the light chain and, particularly, the CDR3 of the heavy chain may constitute the most important determinants in antigen binding within the light and heavy chain variable regions. In some antibodies or antibody constructs/binding domains, the heavy chain CDR3 appears to constitute the major area of contact between the antigen and the antibody. In vitro selection schemes in which CDR3 alone is varied can be used to vary the binding properties of an antibody or antibody construct/binding domain or determine which residues contribute to the binding of an antigen. Hence, CDR3 is typically the greatest source of molecular diversity within the antibody binding site. CDR-H3, for example, can be as short as two amino acid residues or greater than 26 amino acids.

In a classical full-length antibody or immunoglobulin, each light (L) chain is linked to a heavy (H) chain by one covalent disulfide bond, while the two H chains are linked to each other by one or more disulfide bonds depending on the H chain isotype. The heavy chain constant (CH) domain most proximal to VH is usually designated as CH1. The constant (“C”) domains are not directly involved in antigen binding, but exhibit various effector functions, such as antibody-dependent cell-mediated cytotoxicity (ADCC) and complement activation (complement dependent cytotoxicity, CDC). The Fc region of an antibody is the “tail” region of a classical antibody that interacts with cell surface receptors called Fc receptors and some proteins of the complement system. In IgG, IgA and IgD antibody isotypes, the Fc region is composed of two identical protein fragments, derived from the second and third constant domains (CH2 and CH3) of the antibody's two heavy chains. IgM and IgE Fc regions contain three heavy chain constant domains (CH2, CH3 and CH4) in each polypeptide chain. The Fc regions also contains part of the so-called “hinge” region held together by one or more disulfides and noncovalent interactions. The Fc region of a naturally occurring IgG bears a highly conserved N-glycosylation site. Glycosylation of the Fc fragment is essential for Fc receptor-mediated activity.

ADCC is a mechanism of cell-mediated immune defense whereby an effector cell of the immune system actively lyses a target cell, whose membrane-surface antigens have been bound by specific antibodies. ADCC requires an immune effector cell which classically is known to be a natural killer (NK) cell that typically interacts with IgG antibodies. However, ADCC can also be mediated by macrophages, neutrophils and eosinophils. ADCC involves activation of effector cells expressing Fc receptors by antibodies expressing an Fc portion. For example, the most common Fc receptor on the surface of an NK cell is called CD16 or FcγRIII. Once the Fc receptor binds to the Fc region of IgG, the NK cell releases cytotoxic factors that cause the death of the target cell. Likewise, the Fc receptor (FceRI) of an eosinophil will recognize IgE. In CDC, in contrast, the molecule “C1q” of the complement system binds to the antibody Fc region, and this binding triggers the complement cascade which leads to the formation of the membrane attack complex (MAC) at the surface of the target cell, because of the classical pathway complement activation. In therapeutic antibodies or antibody constructs, both ADCC and CDC can be modulated by Fc isotype engineering, Fc genetic mutations, or Fc glycosylation profile modifications.

The sequence of antibody genes after assembly and somatic mutation is highly varied, and these varied genes are estimated to encode 10¹⁰ different antibody molecules (Immunoglobulin Genes, 2^{nd} ed., eds. Jonio et al., Academic Press, San Diego, Calif., 1995). Accordingly, the immune system provides a repertoire of immunoglobulins. The term “repertoire” refers to at least one nucleotide sequence derived wholly or partially from at least one sequence encoding at least one immunoglobulin. The sequence(s) may be generated by rearrangement in vivo of the V, D, and J segments of heavy chains, and the V and J segments of light chains. Alternatively, the sequence(s) can be generated from a cell in response to which rearrangement occurs, e.g., in vitro stimulation. Alternatively, part or all the sequence(s) may be obtained by DNA splicing, nucleotide synthesis, mutagenesis, and other methods, see, e.g., U.S. Pat. No. 5,565,332. A repertoire may include only one sequence or may include a plurality of sequences, including ones in a genetically diverse collection.

It is envisaged that the antibody construct has a cysteine clamp within the first domain. This cysteine clamp may be introduced to improve stability of the construct. See e.g. US 2016/0193295.

As described herein above, the invention provides an embodiment wherein the antibody construct is in a format selected from the group consisting of (scFv)₂, scFv-single domain mAb, diabodies and oligomers of any of the aforementioned formats. The term “is in a format” does not exclude that the construct can be further modified, e.g. by attachment or fusion to other moieties, as described herein. According to one embodiment of the antibody construct used in accordance with the present invention, the first and/or the second domain are in the format of an scFv. In an scFv, the VH region and the and VL region are arranged in the order VH-VL or VL-VH (from N- to C-terminus). It is envisaged that the VH and the VL regions of the first and/or the second binding domain are connected via a linker, preferably a peptide linker. According to one embodiment of the first and/or the second domain, the VH-region is positioned N-terminally of the linker, and the VL-region is positioned C-terminally of the linker. In other words, in one embodiment of the first and/or the second domain, the scFv comprises from the N-terminus to the C-terminus: VH-linker-VL. It is furthermore envisaged that the first domain and the second domain of the antibody construct are connected via a linker, preferably a peptide linker. The antibody construct may e.g. comprise the domains in the order (from N-terminus to C-terminus) first domain-linker-second domain. The inverse order (second domain-linker-first domain) is also possible.

The linkers are preferably peptide linkers, more preferably short peptide linkers. In accordance with the present invention, a “peptide linker” comprises an amino acid sequence which connects the amino acid sequences of one domain with another (variable and/or binding) domain (e.g. a variable domain or a binding domain) of the antibody construct. An essential technical feature of such peptide linker is that it does not comprise any polymerization activity. Among the suitable peptide linkers are those described in U.S. Pat. Nos. 4,751,180 and 4,935,233 or WO 88/09344. The peptide linkers can also be used to attach other domains or modules or regions (such as half-life extending domains) to the antibody construct used in accordance with the invention. Examples of useful peptide linkers are shown in SEQ ID NOs: 202-215. In the present context, a “short” linker has between 2 and 50 amino acids, preferably between 3 and 35, between 4 and 30, between 5 and 25, between 6 and 20 or between 6 and 17 amino acids. The linker between two variable regions of one binding domain may have a different length (e.g. may be longer) than the linker between the two binding domains. For example, the linker between two variable regions of one binding domain may have a length between 7 and 15 amino acids, preferably between 9 and 13, and the linker between the two binding domains may have a length between 3 and 10 amino acids, preferably between 4 and 8. It is further envisaged that the peptide linkers are glycine/serine linkers, such as those depicted in SEQ ID NOs: 203 and 205-215. Most of the amino acids in glycine/serine linkers are selected from glycine and serine.

If a linker is used, this linker is preferably of a length and sequence that are enough to ensure that each of the first and second domains can, independently from one another, retain their differential binding specificities. For peptide linkers which connect the at least two binding domains (or the two variable regions forming one binding domain) in the antibody construct, those peptide linkers are envisaged which comprise only a few amino acid residues, e.g. 12 amino acid residues or less. Thus, peptide linkers of 12, 11, 10, 9, 8, 7, 6 or 5 amino acid residues are preferred. An envisaged peptide linker with less than 5 amino acids comprises 4, 3, 2 or one amino acid(s), wherein Gly-rich linkers are preferred. A “single amino acid” linker in the context of said “peptide linker” is Gly. Another embodiment of a peptide linker is characterized by the amino acid sequence Gly-Gly-Gly-Gly-Ser, i.e. Gly₄Ser (SEQ ID NO: 203), or polymers thereof, i.e. (Gly₄Ser)x, where x is an integer of 1 or greater (e.g. 2 or 3). Usable linkers are depicted in SEQ ID NOs: 202-211. The characteristics of said peptide linkers are known in the art and are described e.g. in Dall'Acqua et al. (Biochem. (1998) 37, 9266-9273), Cheadle et al. (Mol Immunol (1992) 29, 21-30) and Raag and Whitlow (FASEB (1995) 9(1), 73-80). Peptide linkers which do not promote any secondary structures are preferred. The linkage of said domains to each other can be provided, e.g., by genetic engineering. Methods for preparing fused and operatively linked bispecific single chain constructs and expressing them in mammalian cells or bacteria are well-known in the art (e.g. WO 99/54440 or Sambrook et al., Molecular Cloning: A Laboratory Manual, Cold Spring Harbor Laboratory Press, Cold Spring Harbor, N.Y., 2001).

According to one embodiment of the invention, the antibody construct in combination products, or that is used in combination in accordance with the invention is a “single chain antibody construct”. It is also envisaged that either the first or the second or both binding domains may be in the format of a “single chain Fv” (scFv). Although the two domains of the Fv fragment, VL and VH, are coded for by separate genes, they can be joined, using recombinant methods, by an artificial linker—as described hereinbefore—that enables them to be made as a single protein chain in which the VL and VH regions pair to form a monovalent molecule; see e.g., Huston et al. (1988) Proc. Natl. Acad. Sci USA 85:5879-5883). These antibody fragments are obtained using conventional techniques known to those with skill in the art, and the fragments are evaluated for function in the same manner as are full-length antibodies or IgGs. A single-chain variable fragment (scFv) is hence a fusion protein of the variable region of the heavy chain (VH) and of the light chain (VL) of immunoglobulins, usually connected with a short linker peptide. The linker is usually rich in glycine for flexibility, as well as serine or also threonine for solubility, and can either connect the N-terminus of the VH with the C-terminus of the VL, or vice versa. This protein retains the specificity of the original immunoglobulin, despite removal of the constant regions and introduction of the linker.

Bispecific single chain molecules are known in the art and are described in WO 99/54440, Mack, J. Immunol. (1997), 158, 3965-3970, Mack, PNAS, (1995), 92, 7021-7025, Kufer, Cancer Immunol. Immunother., (1997), 45, 193-197, Löffler, Blood, (2000), 95, 6, 2098-2103, Brühl, Immunol., (2001), 166, 2420-2426, Kipriyanov, J. Mol. Biol., (1999), 293, 41-56. Techniques described for producing single chain antibody constructs (see, inter alia, U.S. Pat. No. 4,946,778, Kontermann and Dübel (2010), loc. cit. and Little (2009), loc. cit.) can be adapted to produce single chain antibody constructs specifically recognizing (an) elected target(s).

Bivalent (also called divalent) or bispecific single-chain variable fragments (bi-scFvs or di-scFvs) having the format (scFv)₂ can be engineered by linking two scFv molecules (e.g. with linkers as described hereinbefore). The linking can be done by producing a single polypeptide chain with two VH regions and two VL regions, yielding tandem scFvs (see e.g. Kufer P. et al., (2004) Trends in Biotechnology 22(5):238-244). Another possibility is the creation of scFv molecules with linker peptides that are too short for the two variable regions to fold together (e.g. about five amino acids), forcing the scFvs to dimerize. In this case, the VH and the VL of a binding domain (binding either to the target antigen or to CD3) are not directly connected via a peptide linker. Thus, the VH of the CD3 binding domain may e.g. be fused to the VL of the target antigen(s) binding domain via a peptide linker, and the VH of the target antigen(s) binding domain is fused to the VL of the CD3 binding domain via such peptide linker. This type is known as diabodies (see e.g. Hollinger, Philipp et al., (July 1993) Proceedings of the National Academy of Sciences of the United States of America 90 (14): 6444-8.).

Antibody constructs denominated “single domain antibodies” comprise one (monomeric) antibody variable region which can bind selectively to a specific antigen, independently of other variable regions. The first single domain antibodies were engineered from heavy chain antibodies found in camelids, and these are called V_{H}H fragments. Cartilaginous fishes also have heavy chain antibodies (IgNAR) from which single domain antibodies called V_{NAR} fragments can be obtained. An alternative approach is to split the dimeric variable regions from common immunoglobulins into monomers, hence obtaining VH or VL as a single domain Ab. Although most research into single domain antibodies is currently based on heavy chain variable regions, nanobodies derived from light chains were also shown to bind specifically to target epitopes. Examples of single domain antibodies are called sdAb, nanobodies or single variable domain antibodies. A (single domain mAb)₂ is hence a monoclonal antibody construct composed of (at least) two single domain monoclonal antibody constructs, which are individually selected from the group comprising VH, VL, VHH and VNAR. The linker is preferably in the form of a peptide linker. Similarly, an “scFv-single domain mAb” is a monoclonal antibody construct composed of at least one single domain antibody as described above and one scFv molecule as described above. Again, the linker is preferably in the form of a peptide linker.

It is also envisaged that the antibody construct has, in addition to its function to bind to the target molecules and CD3, a further function. In this format, the antibody construct may be a trifunctional or multifunctional antibody construct by targeting target cells through target antigen binding, mediating cytotoxic T cell activity through CD3 binding and providing a further function such as means or domains to enhance or extend serum half-life, a fully functional or modified Fc constant domain mediating ADCC through recruitment of effector cells, a label (fluorescent etc.), a therapeutic agent such as a toxin or radionuclide, etc.

Examples for means or domains to extend serum half-life of the antibody constructs include peptides, proteins or domains of proteins, which are fused or otherwise attached to the antibody constructs. The group of peptides, proteins or protein domains includes peptides binding to other proteins with preferred pharmacokinetic profile in the human body such as serum albumin (see WO 2009/127691). An alternative concept of such half-life extending peptides includes peptides binding to the neonatal Fc receptor (FcRn, see WO 2007/098420), which can also be used in the antibody constructs used in accordance with the present invention. The concept of attaching larger domains of proteins or complete proteins includes the fusion of human serum albumin, variants or mutants of human serum albumin (see WO 2011/051489, WO 2012/059486, WO 2012/150319, WO 2013/135896, WO 2014/072481, WO 2013/075066) or domains thereof, as well as the fusion of an immunoglobulin constant region (Fc domain) and variants thereof. Such variants of Fc domains are called Fc-based domains and may e.g. be optimized/modified to allow the desired pairing of dimers or multimers, to abolish Fc receptor binding (e.g. to avoid ADCC or CDC) or for other reasons. A further concept known in the art to extend the half-life of substances or molecules in the human body is the pegylation of those molecules (such as the antibody constructs used in accordance with the present invention).

In one embodiment, the antibody constructs used in accordance with the invention are linked (e.g. via peptide bond) with a fusion partner (such as a protein, polypeptide or peptide), e.g. for extending the construct's serum half-life. These fusion partners can be selected from human serum albumin (“HSA” or “HALB”) as wells as sequence variants thereof, peptides binding to HSA, peptides binding to FcRn (“FcRn BP”), or constructs comprising an (antibody derived) Fc region. Exemplary sequences of these fusion partners are depicted in SEQ ID NOs: 216-278. In general, the fusion partners may be linked to the N-terminus or to the C-terminus of the antibody constructs used in accordance with the invention, either directly (e.g. via peptide bond) or through a peptide linker such as (GGGGS)ₙ (wherein “n” is an integer of 2 or greater, e.g. 2 or 3 or 4). Suitable peptide linkers are depicted in SEQ ID NOs: 202-211.

According to another embodiment, the antibody construct used in accordance with the invention comprises (in addition to the first and second domain) a third domain which comprises two polypeptide monomers, each comprising a hinge, a CH2 and a CH3 domain, wherein said two polypeptide monomers are fused to each other via a peptide linker. It is envisaged that said third domain comprises in N-terminal to C-terminal order: hinge-CH2-CH3-linker-hinge-CH2-CH3.

In line with the present invention, a “hinge” is an IgG hinge region. This region can be identified by analogy using the Kabat numbering, see e.g. Kabat positions 223-243. In line with the above, the minimal requirement for a “hinge” are the amino acid residues corresponding to the IgG₁ sequence stretch of D231 to P243 according to the Kabat numbering. The terms “CH2” and “CH3” refer to the immunoglobulin heavy chain constant regions 2 and 3. These regions can as well be identified by analogy using the Kabat numbering, see e.g. Kabat positions 244-360 for CH2 and Kabat positions 361-478 for CH3. It is understood that there is some variation between the immunoglobulins in terms of their IgG₁ Fc region, IgG₂ Fc region, IgG₃ Fc region, IgG₄ Fc region, IgM Fc region, IgA Fc region, IgD Fc region and IgE Fc region (see, e.g., Padlan, Molecular Immunology, 31(3), 169-217 (1993)). The term Fc region refers to the last two heavy chain constant regions of IgA, IgD, and IgG, and the last three heavy chain constant regions of IgE and IgM. The Fc region can also include the flexible hinge N-terminal to these domains. For IgA and IgM, the Fc region may include the J chain. For IgG, the Fc region comprises immunoglobulin domains CH2 and CH3 and the hinge between the first two domains and CH2. Although the boundaries of the Fc region of an immunoglobulin may vary, an example for a human IgG heavy chain Fc portion comprising a functional hinge, CH2 and CH3 domain can be defined e.g. to comprise residues D231 (of the hinge domain) to P476 (of the C-terminus of the CH3 domain), or D231 to L476, respectively, for IgG₄, wherein the numbering is according to Kabat.

Covalent modifications of the antibody constructs are also included within the scope of this invention, and are generally, but not always, done post-translationally. For example, several types of covalent modifications of the antibody construct are introduced into the molecule by reacting specific amino acid residues of the antibody construct with an organic derivatizing agent that can react with selected side chains or with the N- or C-terminal residues. Derivatization with bifunctional agents is useful for crosslinking the antibody constructs used in accordance with the present invention to a water-insoluble support matrix or surface used in a variety of methods. Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention. Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, 1983, pp. 79-86), acetylation of the N-terminal amine, and amidation of any C-terminal carboxyl group.

Another type of covalent modification of the antibody constructs included within the scope of this invention comprises altering the glycosylation pattern of the protein. As is known in the art, glycosylation patterns can depend on both the sequence of the protein (e.g., the presence or absence of specific glycosylation amino acid residues, discussed below), or the host cell or organism in which the protein is produced. Specific expression systems are discussed below. Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Addition of glycosylation sites to the antibody construct is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the amino acid sequence of an antibody construct may be altered through changes at the DNA level, particularly by mutating the DNA encoding the polypeptide at preselected bases such that codons are generated that will translate into the desired amino acids.

Another means of increasing the number of carbohydrate moieties on the antibody construct is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330, and in Aplin and Wriston, 1981, CRC Crit. Rev. Biochem., pp. 259-306.

Removal of carbohydrate moieties present on the starting antibody construct may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118:131. Enzymatic cleavage of carbohydrate moieties on polypeptides can be achieved using a variety of endo- and exo-glycosidases as described by Thotakura et al., 1987, Meth. Enzymol. 138:350. Glycosylation at potential glycosylation sites may be prevented using the compound tunicamycin as described by Duskin et al., 1982, J. Biol. Chem. 257:3105. Tunicamycin blocks the formation of protein-N-glycoside linkages.

Other modifications of the antibody construct are also contemplated herein. For example, another type of covalent modification of the antibody construct comprises linking the antibody construct to various non-proteinaceous polymers, including polyols, in the manner set forth in U.S. Pat. Nos. 4,640,835; 4,496,689; 4,301,144; 4,670,417; 4,791,192 or 4,179,337. In addition, as is known in the art, amino acid substitutions may be made in various positions within the antibody construct, e.g. to facilitate the addition of polymers such as polyethylene glycol (PEG).

In some embodiments, the covalent modification of the antibody constructs used in accordance with the invention comprises the addition of one or more labels. The labelling group may be coupled to the antibody construct via spacer arms of various lengths to reduce potential steric hindrance. Various methods for labelling proteins are known in the art and can be used in performing the present invention. The term “label” or “labelling group” refers to any detectable label. In general, labels fall into a variety of classes, depending on the assay in which they are to be detected—the following examples include, but are not limited to:
a) isotopic labels, which may be radioactive or heavy isotopes, such as radioisotopes or radionuclides (e.g., ³H, ¹⁴C, ¹⁵N, ³⁵S, ⁸⁹Zr, ⁹⁰Y, ⁹⁹Tc, ¹¹¹In, ¹²⁵I, ¹³¹I)
b) magnetic labels (e.g., magnetic particles)
c) redox active moieties
d) optical dyes (including, but not limited to, chromophores, phosphors and fluorophores) such as fluorescent groups (e.g., FITC, rhodamine, lanthanide phosphors), chemiluminescent groups, and fluorophores which can be either “small molecule” fluores or proteinaceous fluores
e) enzymatic groups (e.g. horseradish peroxidase, β-galactosidase, luciferase, alkaline phosphatase)
f) biotinylated groups
g) predetermined polypeptide epitopes recognized by a secondary reporter (e.g., leucine zipper pair sequences, binding sites for secondary antibodies, metal binding domains, epitope tags, etc.)

By “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade BlueJ, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, Oreg.), FITC, Rhodamine, and Texas Red (Pierce, Rockford, Ill.), Cy5, Cy5.5, Cy7 (Amersham Life Science, Pittsburgh, Pa.). Suitable optical dyes, including fluorophores, are described in Molecular Probes Handbook by Richard P. Haugland.

Suitable proteinaceous fluorescent labels also include, but are not limited to, green fluorescent protein, including a Renilla, Ptilosarcus, or Aequorea species of GFP (Chalfie et al., 1994, Science 263:802-805), EGFP (Clontech Laboratories, Inc., Genbank® Accession Number U55762), blue fluorescent protein (BFP, Quantum Biotechnologies, Inc. 1801 de Maisonneuve Blvd. West, 8th Floor, Montreal, Quebec, Canada H3H 1J9; Stauber, 1998, Biotechniques 24:462-471; Heim et al., 1996, Curr. Biol. 6:178-182), enhanced yellow fluorescent protein (EYFP, Clontech Laboratories, Inc.), luciferase (Ichiki et al., 1993, J. Immunol. 150:5408-5417), β galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658; 5,418,155; 5,683,888; 5,741,668; 5,777,079; 5,804,387; 5,874,304; 5,876,995; 5,925,558).

Leucine zipper domains are peptides that promote oligomerization of the proteins in which they are found. Leucine zippers were originally identified in several DNA-binding proteins (Landschulz et al., 1988, Science 240:1759), and have since been found in a variety of different proteins. Among the known leucine zippers are naturally occurring peptides and derivatives thereof that dimerize or trimerize. Examples of leucine zipper domains suitable for producing soluble oligomeric proteins are described in PCT application WO94/10308, and the leucine zipper derived from lung surfactant protein D (SPD) described in Hoppe et al., 1994, FEBS Letters 344:191. The use of a modified leucine zipper that allows for stable trimerization of a heterologous protein fused thereto is described in Fanslow et al., 1994, Semin. Immunol. 6:267-78.

The antibody construct used in accordance with the invention may also comprise additional domains, which are e.g. helpful in the isolation of the molecule or relate to an adapted pharmacokinetic profile of the molecule. Domains helpful for the isolation of an antibody construct may be selected from peptide motives or secondarily introduced moieties, which can be captured in an isolation method, e.g. an isolation column. Non-limiting embodiments of such additional domains comprise peptide motives known as Myc-tag, HAT-tag, HA-tag, TAP-tag, GST-tag, chitin binding domain (CBD-tag), maltose binding protein (MBP-tag), Flag-tag, Strep-tag and variants thereof (e.g. StrepII-tag) and His-tag. All herein disclosed antibody constructs characterized by the identified CDRs may comprise a His-tag domain, which is generally known as a repeat of consecutive His residues in the amino acid sequence of a molecule, e.g. of five His residues (SEQ ID NO: 279), or of six His residues (hexa-histidine, SEQ ID NO: 280). The His-tag may be located e.g. at the N- or C-terminus of the antibody construct. In one embodiment, a hexa-histidine tag (HHHHHH (SEQ ID NO: 280) is linked via peptide bond to the C-terminus of the antibody construct used in accordance with the invention.

It is also envisaged that the antibody construct used in accordance with the present invention comprises or consists of a polypeptide which has an amino acid sequence selected from the group consisting of those depicted in SEQ ID NOs: 281 and 282, and which is linked at its N-terminus or at its C-terminus with a protein purification tag, preferably via a peptide bond (amide bond). The linking of the protein purification tag at the C-terminus of the polypeptide is preferred. It is envisaged that the protein purification tag is a short peptide. For example, the length of the short peptide may be 2-30 amino acids, 4-25 amino acids, 5-20 amino acids or 6-19 amino acids. Examples of protein purification tags include, but are not limited to, AU1 epitope (e.g. as depicted in SEQ ID NO: 285), AU5 epitope (e.g. as depicted in SEQ ID NO: 286), T7-tag (e.g. as depicted in SEQ ID NO: 287), V5-tag (e.g. as depicted in SEQ ID NO: 288), B-tag (e.g. as depicted in SEQ ID NO: 289), E2 epitope (e.g. as depicted in SEQ ID NO: 290), FLAG epitope/FLAG tag (e.g. as depicted in SEQ ID NO: 291), Glu-Glu tag (e.g. as depicted in SEQ ID NOs: 292 or 293), HA tag, Histidine affinity tag (e.g. as depicted in SEQ ID NO: 294), HSV epitope (e.g. as depicted in SEQ ID NO: 295), KT3 epitope (e.g. as depicted in SEQ ID NO: 296), Myc epitope (e.g. as depicted in SEQ ID NO: 297), polyarginine tag (5-6 Arg residues), polyaspartate tag (5-16 Asp residues), polyhistidine tag (2-10 His residues, usually 6 His residues, see e.g. SEQ ID NOs: 280, and (279, 298, 299-), polyphenylalanine tag (usually 11 Phe residues), 51 tag (e.g. as depicted in SEQ ID NO: 300), S-tag (e.g. as depicted in SEQ ID NO: 301), Strep-tag (e.g. as depicted in SEQ ID NOs: 302 or 303), universal tag (e.g. as depicted in SEQ ID NO: 304), VSV-G (e.g. as depicted in SEQ ID NO: 305), Protein C (e.g. as depicted in SEQ ID NO: 306), and Protein A. A histidine tag is preferred, especially a 6×His tag (SEQ ID NO: 280). Is it hence further envisaged that the antibody construct used in accordance with the present invention consists of a polypeptide which has an amino acid sequence selected from the group consisting of those depicted in SEQ ID NOs: 281 and 282, and which is linked at its C-terminus with a 6×His tag via a peptide bond. An embodiment of the antibody construct used in accordance with the present invention has an amino acid sequence as depicted in SEQ ID NO: 283 or SEQ ID NO: 284.

T cells or T lymphocytes are a type of lymphocyte (itself a type of white blood cell) that play a central role in cell-mediated immunity. There are several subsets of T cells, each with a distinct function. T cells can be distinguished from other lymphocytes, such as B cells and NK cells, by the presence of a T cell receptor (TCR) on the cell surface. The TCR is responsible for recognizing antigens bound to major histocompatibility complex (MHC) molecules and is composed of two different protein chains. In 95% of the T cells, the TCR consists of an alpha (α) and beta (β) chain. When the TCR engages with antigenic peptide and MHC (peptide/MHC complex), the T lymphocyte is activated through a series of biochemical events mediated by associated enzymes, co-receptors, specialized adaptor molecules, and activated or released transcription factors.

The antibody construct used in accordance with the invention comprises a domain which binds to CD3 on the surface of a T cell. “CD3” (cluster of differentiation 3) is a T cell co-receptor composed of four chains. In mammals, the CD3 protein complex contains a CD3γ (gamma) chain, a CD3δ (delta) chain, and two CD3ε (epsilon) chains. These four chains associate with the T cell receptor (TCR) and the so-called ζ (zeta) chain to for the “T cell receptor complex” and to generate an activation signal in T lymphocytes. The CD3γ (gamma), CD3δ (delta), and CD3ε (epsilon) chains are highly related cell-surface proteins of the immunoglobulin superfamily and each contain a single extracellular immunoglobulin domain. The intracellular tails of the CD3 molecules contain a single conserved motif known as an immunoreceptor tyrosine-based activation motif (ITAM), which is essential for the signalling capacity of the TCR. The CD3 epsilon molecule is a polypeptide which in humans is encoded by the CD3E gene which resides on chromosome 11.

The redirected lysis of target cells via the recruitment of T cells by an antibody construct which binds to CD3 on the T cell and to a target protein on the target cell generally involves cytolytic synapse formation and delivery of perforin and granzymes. The engaged T cells are capable of serial target cell lysis and are not affected by immune escape mechanisms interfering with peptide antigen processing and presentation, or clonal T cell differentiation; see e.g. WO2007/042261.

Cytotoxicity mediated by the herein described antibody constructs can be measured in various ways. The “half maximal effective concentration” (EC₅₀) is commonly used as a measure of potency of a biologically active molecule such as an antibody construct used in accordance with the present invention. It may be expressed in molar units. In the present case of measuring cytotoxicity, the EC₅₀ value refers to the concentration of an antibody construct inducing a cytotoxic response (lysis of target cells) halfway between the baseline and the maximum. Effector cells in a cytotoxicity assay can e.g. be stimulated enriched (human) CD8 positive T cells or unstimulated (human) peripheral blood mononuclear cells (PBMC). An EC₅₀ value may typically be expected to be lower when stimulated/enriched CD8+ T cells are used as effector cells, compared with unstimulated PBMC. If the target cells are of macaque origin or express or are transfected with macaque target antigen, the effector cells should also be of macaque origin, such as a macaque T cell line, e.g. 4119LnPx. The target cells should express the target antigen(s), such as human or macaque the target antigen(s), on the cell surface. Target cells can be a cell line (such as CHO) which is stably or transiently transfected with nucleic acids encoding the target antigen(s). Alternatively, the target cells can be a target antigen(s)-positive natural expresser cell line, such as the human cancer lines. Usually EC₅₀ values are expected to be lower when using target cells that express higher levels of the target antigen(s) on the cell surface compared with target cells having a lower target expression rate.

The effector to target cell (E:T) ratio in a cytotoxicity assay is usually about 10:1, but can also vary. Cytotoxic activity of the target antigen(s)×CD3 antibody constructs can be measured in a 51-chromium release assay (e.g. with an incubation time of about 18 hours) or in a in a FACS-based cytotoxicity assay (e.g. with an incubation time of about 48 hours). Modifications of the incubation time (cytotoxic reaction) are also envisaged. Other methods of measuring cytotoxicity are well-known and comprise MTT or MTS assays, ATP-based assays including bioluminescent assays, the sulforhodamine B (SRB) assay, WST assay, clonogenic assay and the ECIS technology.

According to one embodiment, the cytotoxic activity mediated by the target antigen(s)×CD3 antibody constructs used in accordance with the present invention is measured in a cell-based cytotoxicity assay. It may also be measured in a 51-chromium release assay. It is envisaged that the EC₅₀ value of the antibody constructs used in accordance with the invention is ≤300 pM, ≤280 pM, ≤260 pM, ≤250 pM, ≤240 pM, ≤220 pM, ≤200 pM, ≤180 pM, ≤160 pM, ≤150 pM, ≤140 pM, ≤120 pM, ≤100 pM, ≤90 pM, ≤80 pM, ≤70 pM, ≤60 pM, ≤50 pM, ≤40 pM, ≤30 pM, ≤20 pM, ≤15 pM, ≤10 pM, or ≤5 pM.

The above given EC₅₀ values can be measured in different assays and under different conditions. For example, when human PBMCs are used as effector cells and the target antigen(s)-transfected cells such as CHO cells are used as target cells, it is envisaged that the EC₅₀ value of the antibody construct is ≤500 pM, ≤400 pM, ≤300 pM, ≤280 pM, ≤260 pM, ≤250 pM, ≤240 pM, ≤220 pM, ≤200 pM, ≤180 pM, ≤160 pM, ≤150 pM, ≤140 pM, ≤120 pM, ≤100 pM, ≤90 pM, ≤80 pM, ≤70 pM, ≤60 pM, ≤50 pM, ≤40 pM, ≤30 pM, ≤20 pM, ≤15 pM, ≤10 pM, or ≤5 pM. When human PBMCs are used as effector cells and when the target cells are a the target antigen(s) positive cell line such as, it is envisaged that the EC₅₀ value of the target antigen(s)×CD3 antibody construct is ≤300 pM, ≤280 pM, ≤260 pM, ≤250 pM, ≤240 pM, ≤220 pM, ≤200 pM, ≤180 pM, ≤160 pM, ≤150 pM, ≤140 pM, ≤120 pM, ≤100 pM, ≤90 pM, ≤80 pM, ≤70 pM, ≤60 pM, ≤50 pM, ≤40 pM, ≤30 pM, ≤20 pM, ≤15 pM, ≤10 pM, or ≤5 pM.

According to one embodiment, the target antigen(s)×CD3 antibody constructs used in accordance with the present invention do not induce/mediate lysis or do not essentially induce/mediate lysis of cells that do not express the target antigen(s) on their surface (the target antigen(s)-negative cells), such as CHO cells. The term “do not induce lysis”, “do not essentially induce lysis”, “do not mediate lysis” or “do not essentially mediate lysis” means that an antibody construct used in accordance with the present invention does not induce or mediate lysis of more than 30%, preferably not more than 20%, more preferably not more than 10%, particularly preferably not more than 9%, 8%, 7%, 6% or 5% of the target antigen(s)-negative cells, whereby lysis of the target antigen(s) expressing target cells (such as cells transformed or transfected with the target antigen(s) or a natural expresser cell line such as human cancer lines) is set to be 100%. This usually applies for concentrations of the antibody construct of up to 500 nM. Cell lysis measurement is a routine technique. Moreover, the present specification teaches specific instructions how to measure cell lysis.

The difference in cytotoxic activity between the monomeric and the dimeric isoform of individual the target antigen(s)×CD3 antibody constructs is referred to as “potency gap”. This potency gap can e.g. be calculated as ratio between EC₅₀ values of the molecule's monomeric and dimeric form. In one method to determine this gap, an 18 hour 51-chromium release assay or a 48 h FACS-based cytotoxicity assay is carried out as described known in the art with purified antibody construct monomer and dimer. Effector cells are stimulated enriched human CD8+ T cells or unstimulated human PBMC. Target cells are target antigen(s)-transfected CHO cells. Effector to target cell (E:T) ratio is 10:1. Potency gaps of the target antigen(s)×CD3 antibody constructs used in accordance with the present invention are preferably ≤5, more preferably ≤4, even more preferably ≤3, even more preferably ≤2 and most preferably ≤1.

The first and/or the second domain of the antibody construct used in accordance with the invention is/are preferably cross-species specific for members of the mammalian order of primates, such as macaques. Cross-species specific CD3 binding domains are, for example, described in WO 2008/119567. According to one embodiment, the second domain, in addition to binding to human CD3, will also bind to CD3 of primates including (but not limited to) new world primates (such as Callithrix jacchus, Saguinus Oedipus or Saimiri sciureus), old world primates (such as baboons and macaques), gibbons, orangutans and non-human homininae. It is envisaged that the second domain which binds to human CD3 on the surface of a T cell also binds at least to macaque CD3. A preferred macaque is Macaca fascicularis. Macaca mulatta (Rhesus) is also envisaged. One antibody construct used in accordance with the invention comprises a first domain which binds to human the target antigen(s) on the surface of a target cell and a second domain which binds to human CD3 on the surface of a T cell and at least macaque CD3.

In one embodiment, the affinity gap of the antibody constructs used in accordance with the invention for binding macaque CD3 versus human CD3 [KD ma CD3:KD hu CD3] (as determined e.g. by BiaCore or by Scatchard analysis) is between 0.01 and 100, preferably between 0.1 and 10, more preferably between 0.2 and 5, more preferably between 0.3 and 4, even more preferably between 0.5 and 3 or between 0.5 and 2.5, and most preferably between 0.5 and 1.

The second domain of the antibody construct used in accordance with the invention binds to CD3. More preferably, it binds to CD3 on the surface of a T cell. It is furthermore envisaged that the second domain binds to human CD3, preferably to human CD3 on the surface of a T cell. It is also envisaged that the second domain binds to CD3 epsilon. More preferably, it binds to human CD3 epsilon, e.g. to human CD3 epsilon on the surface of a T cell. A preferred amino acid sequence for the extracellular domain of human CD3 epsilon is depicted in SEQ ID NO: 1.

In one embodiment used in accordance with the present invention, the second domain of the antibody construct binds to human CD3 epsilon (or human CD3 epsilon on the surface of a T cell) and to Callithrix jacchus or Saimiri sciureus CD3 epsilon. It is also envisaged that the second domain binds to an extracellular epitope of CD3 epsilon, preferably to an extracellular epitope of human CD3 epsilon. It is also envisaged that the second domain binds to an extracellular epitope of the human and the Macaca CD3 epsilon chain. One preferred epitope of CD3 epsilon is comprised within amino acid residues 1-27 of the human CD3 epsilon extracellular domain (see SEQ ID NO: 2). Even more specifically, the epitope comprises at least the amino acid sequence Gln-Asp-Gly-Asn-Glu (SEQ ID NO: 307). Callithrix jacchus is a new world primate belonging to the family of Callitrichidae, while Saimiri sciureus is a new world primate belonging to the family of Cebidae. Binders having such characteristics are described in detail in WO 2008/119567.

Antibodies or bispecific antibody constructs directed against (human) CD3 or specifically against CD3 epsilon are known in the art, and their CDRs, VH and VL sequences can serve as a basis for the second binding domain of the antibody construct used in accordance with the invention. For example, Kung et al. reported in 1979 the development of OKT3 (Ortho Kung T3), the first mAb recognizing CD3 (specifically, the epsilon chain of CD3) on human T cells. OKT3 (muromonab) was the first monoclonal antibody of murine origin to become available for therapy in humans. Newer anti-CD3 monoclonal antibodies include otelixizumab (TRX4), teplizumab (MGA031), foralumab and visilizumab, all targeting the epsilon chain of CD3. Bispecific antibody constructs directed against a (cancer) target and CD3 are also being developed and (pre-)clinically tested, and their CD3 binding domain (CDRs, VH, VL) may serve as a basis for the second binding domain of the antibody construct used in accordance with the invention. Examples include, but are not limited to, Blinatumomab, Solitomab (MT110, AMG 110), Catumaxomab, Duvortuxizumab, Ertumaxomab, Mosunetuzumab, FBTA05 (Bi20, TPBs05), CEA-TCB (RG7802, RO6958688), AFM11, and MGD006 (S80880). Other examples of CD3 binding domains are disclosed e.g. in U.S. Pat. No. 7,994,289 B2, U.S. Pat. No. 7,728,114 B2, U.S. Pat. No. 7,381,803 B1, U.S. Pat. No. 6,706,265 B1.

It is envisaged for the antibody construct used in accordance with the present invention that the second domain which binds to CD3 on the surface of a T cell comprises a VL region comprising CDR-L1, CDR-L2 and CDR-L3 selected from:
(a) CDR-L1 as depicted in SEQ ID NO: 3, CDR-L2 as depicted in SEQ ID NO: 4, and CDR-L3 as depicted in SEQ ID NO: 5;
(b) CDR-L1 as depicted in SEQ ID NO: 6, CDR-L2 as depicted in SEQ ID NO: 7, and CDR-L3 as depicted in SEQ ID NO: 8; and
(c) CDR-L1 as depicted in SEQ ID NO: 9, CDR-L2 as depicted in SEQ ID NO: 10, and CDR-L3 as depicted in SEQ ID NO: 11.

It is also envisaged for the antibody construct used in accordance with the present invention that the second domain which binds to CD3 on the surface of a T cell comprises a VH region comprising CDR-H1, CDR-H2 and CDR-H3 selected from:
(a) CDR-H1 as depicted in SEQ ID NO: 12, CDR-H2 as depicted in SEQ ID NO: 13, and CDR-H3 as depicted in SEQ ID NO: 14;
(b) CDR-H1 as depicted in SEQ ID NO: 15, CDR-H2 as depicted in SEQ ID NO: 16, and CDR-H3 as depicted in SEQ ID NO: 17;
(c) CDR-H1 as depicted in SEQ ID NO: 18, CDR-H2 as depicted in SEQ ID NO: 19, and CDR-H3 as depicted in SEQ ID NO: 20;
(d) CDR-H1 as depicted in SEQ ID NO: 21, CDR-H2 as depicted in SEQ ID NO: 22, and CDR-H3 as depicted in SEQ ID NO: 23;
(e) CDR-H1 as depicted in SEQ ID NO: 24, CDR-H2 as depicted in SEQ ID NO: 25, and CDR-H3 as depicted in SEQ ID NO: 26;
(f) CDR-H1 as depicted in SEQ ID NO: 27, CDR-H2 as depicted in SEQ ID NO: 28, and CDR-H3 as depicted in SEQ ID NO: 29;
(g) CDR-H1 as depicted in SEQ ID NO: 30, CDR-H2 as depicted in SEQ ID NO: 31, and CDR-H3 as depicted in SEQ ID NO: 32;
(h) CDR-H1 as depicted in SEQ ID NO: 33, CDR-H2 as depicted in SEQ ID NO: 34, and CDR-H3 as depicted in SEQ ID NO: 35;
(i) CDR-H1 as depicted in SEQ ID NO: 36, CDR-H2 as depicted in SEQ ID NO: 37, and CDR-H3 as depicted in SEQ ID NO: 38; and
(j) CDR-H1 as depicted in SEQ ID NO: 39, CDR-H2 as depicted in SEQ ID NO: 40, and CDR-H3 as depicted in SEQ ID NO: 41.

It is furthermore envisaged for the antibody construct used in accordance with the present invention that the second domain which binds to CD3 comprises a VL region comprising CDR-L1, CDR-L2 and CDR-L3 and a VH region comprising CDR-H1, CDR-H2 and CDR-H3 selected from:
(a) CDR-L1 as depicted in SEQ ID NO: 42, CDR-L2 as depicted in SEQ ID NO: 43, CDR-L3 as depicted in SEQ ID NO: 44, CDR-H1 as depicted in SEQ ID NO: 12, CDR-H2 as depicted in SEQ ID NO: 13, and CDR-H3 as depicted in SEQ ID NO: 14;
(b) CDR-L1 as depicted in SEQ ID NO: 3, CDR-L2 as depicted in SEQ ID NO: 4, CDR-L3 as depicted in SEQ ID NO: 5, CDR-H1 as depicted in SEQ ID NO: 15, CDR-H2 as depicted in SEQ ID NO: 16, and CDR-H3 as depicted in SEQ ID NO: 17;
(c) CDR-L1 as depicted in SEQ ID NO: 45, CDR-L2 as depicted in SEQ ID NO: 46, CDR-L3 as depicted in SEQ ID NO: 47, CDR-H1 as depicted in SEQ ID NO: 48, CDR-H2 as depicted in SEQ ID NO: 49, and CDR-H3 as depicted in SEQ ID NO: 50;
(d) CDR-L1 as depicted in SEQ ID NO: 51, CDR-L2 as depicted in SEQ ID NO: 52, CDR-L3 as depicted in SEQ ID NO: 53, CDR-H1 as depicted in SEQ ID NO: 21, CDR-H2 as depicted in SEQ ID NO: 22, and CDR-H3 as depicted in SEQ ID NO: 23;
(e) CDR-L1 as depicted in SEQ ID NO: 54, CDR-L2 as depicted in SEQ ID NO: 55, CDR-L3 as depicted in SEQ ID NO: 56, CDR-H1 as depicted in SEQ ID NO: 24, CDR-H2 as depicted in SEQ ID NO: 25, and CDR-H3 as depicted in SEQ ID NO: 26;
(f) CDR-L1 as depicted in SEQ ID NO: 57, CDR-L2 as depicted in SEQ ID NO: 58, CDR-L3 as depicted in SEQ ID NO: 59, CDR-H1 as depicted in SEQ ID NO: 27, CDR-H2 as depicted in SEQ ID NO: 28, and CDR-H3 as depicted in SEQ ID NO: 29;
(g) CDR-L1 as depicted in SEQ ID NO: 6, CDR-L2 as depicted in SEQ ID NO: 7, CDR-L3 as depicted in SEQ ID NO: 8, CDR-H1 as depicted in SEQ ID NO: 30, CDR-H2 as depicted in SEQ ID NO: 31, and CDR-H3 as depicted in SEQ ID NO: 32;
(h) CDR-L1 as depicted in SEQ ID NO: 60, CDR-L2 as depicted in SEQ ID NO: 61, CDR-L3 as depicted in SEQ ID NO: 62, CDR-H1 as depicted in SEQ ID NO: 33, CDR-H2 as depicted in SEQ ID NO: 34, and CDR-H3 as depicted in SEQ ID NO: 35;
(i) CDR-L1 as depicted in SEQ ID NO: 9, CDR-L2 as depicted in SEQ ID NO: 10, CDR-L3 as depicted in SEQ ID NO: 11, CDR-H1 as depicted in SEQ ID NO: 36, CDR-H2 as depicted in SEQ ID NO: 37, and CDR-H3 as depicted in SEQ ID NO: 38; and
(j) CDR-L1 as depicted in SEQ ID NO: 63, CDR-L2 as depicted in SEQ ID NO: 64, CDR-L3 as depicted in SEQ ID NO: 65, CDR-H1 as depicted in SEQ ID NO: 39, CDR-H2 as depicted in SEQ ID NO: 40, and CDR-H3 as depicted in SEQ ID NO: 41.

It is envisaged for the antibody construct used in accordance with the present invention that the second domain which binds to CD3 on the surface of a T cell comprises a VL region selected from the group consisting of a VL region as depicted in any one of SEQ ID NO: 66, SEQ ID NO: 67, SEQ ID NO: 68, SEQ ID NO: 69, SEQ ID NO: 70 and SEQ ID NO: 71.

It is also envisaged that the second domain which binds to CD3 on the surface of a T cell comprises a VH region selected from the group consisting of a VH region as depicted in any one of SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, and SEQ ID NO: 92.

More preferably, the antibody construct used in accordance with the present invention is characterized by the second domain which binds to CD3 on the surface of a T cell comprising a VL region and a VH region selected from the group consisting of:
(a) a VL region as depicted in SEQ ID NO: 93 or 69 and a VH region as depicted in SEQ ID NO: 72 or 83;
(b) a VL region as depicted in SEQ ID NO: 66 or 69 and a VH region as depicted in SEQ ID NO: 73 or 84;
(c) a VL region as depicted in SEQ ID NO: 94 or 69 and a VH region as depicted in SEQ ID NO: 74 or 85;
(d) a VL region as depicted in SEQ ID NO: 95 or 69 and a VH region as depicted in SEQ ID NO: 75 or 86;
(e) a VL region as depicted in SEQ ID NO: 96 or 70 and a VH region as depicted in SEQ ID NO: 76 or 87;
(f) a VL region as depicted in SEQ ID NO: 97 or 69 and a VH region as depicted in SEQ ID NO: 77 or 88;
(g) a VL region as depicted in SEQ ID NO: 67 or 70 and a VH region as depicted in SEQ ID NO: 78 or 89;
(h) a VL region as depicted in SEQ ID NO: 98 or 69 and a VH region as depicted in SEQ ID NO: 79 or 90;
(i) a VL region as depicted in SEQ ID NO: 68 or 71 and a VH region as depicted in SEQ ID NO: 80 or 91;
(j) a VL region as depicted in SEQ ID NO: 99 or 71 and a VH region as depicted in SEQ ID NO: 81 or 92; and
(k) a VL region as depicted in SEQ ID NO: 100 and a VH region as depicted in SEQ ID NO: 82.

A preferred embodiment of the above described antibody construct used in accordance with the present invention is characterized by the second domain which binds to CD3 on the surface of a T cell comprising an amino acid sequence selected from the group consisting of SEQ ID NOs: 101, 102, 103, 104, 105, 106, 107, 108, 109, 110, 111, 112, 113, 114, 115, 116, 117, 118, 119, 120 and 121.

Amino acid sequence modifications of the antibody constructs described herein are also contemplated. For example, it may be desirable to improve the binding affinity and/or other biological properties of the antibody construct Amino acid sequence variants of the antibody constructs are prepared by peptide synthesis or by introducing appropriate nucleotide changes into the nucleic acid molecule encoding the antibody constructs. All below described amino acid sequence modifications should result in an antibody construct which retains the desired biological activity of the unmodified parental molecule (such as binding to the target antigen(s) and to CD3, inducing cytotoxicity against the target antigen(s)-positive target cells).

The term “amino acid” or “amino acid residue” typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (Ile or I): leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V), although modified, synthetic, or rare amino acids may be used as desired. There are basically four different classes of amino acids determined by different side chains:

(1) non-polar and neutral (uncharged): Ala, Gly, Ile, Leu, Met, Phe, Pro, Val
(2) polar and neutral (uncharged): Asn, Cys (being only slightly polar), Gln, Ser, Thr, Trp (being only slightly polar), Tyr
(3) acidic and polar (negatively charged): Asp and Glu
(4) basic and polar (positively charged): Arg, His, Lys

Hydrophobic amino acids can be divided according to whether they have aliphatic or aromatic side chains. Phe and Trp (being very hydrophobic), Tyr and His (being less hydrophobic) are classified as aromatic amino acids. Strictly speaking, aliphatic means that the side chain contains only hydrogen and carbon atoms. By this strict definition, the amino acids with aliphatic side chains are alanine, isoleucine, leucine (also norleucine), proline and valine. Alanine's side chain, being very short, means that it is not particularly hydrophobic, and proline has an unusual geometry that gives it special roles in proteins. It is often convenient to consider methionine in the same category as isoleucine, leucine and valine, although it also contains a sulphur atom. The unifying theme is that these amino acids contain largely non-reactive and flexible side chains. The amino acids alanine, cysteine, glycine, proline, serine and threonine are often grouped together because they are all small. Gly and Pro may influence chain orientation.

Amino acid modifications include, for example, deletions of residues from, insertions of residues into, and/or substitutions of residues within the amino acid sequences of the antibody constructs. Any combination of deletion, insertion, and/or substitution is made to arrive at a final antibody construct, provided that the final construct possesses the desired characteristics, e.g. the biological activity of the unmodified parental molecule (such as binding to the target antigen(s) and to CD3, inducing cytotoxicity against the target antigen(s) positive target cells). The amino acid changes may also alter post-translational processes of the antibody constructs, such as changing the number or position of glycosylation sites.

For example, 1, 2, 3, 4, 5, or 6 amino acids may be inserted, deleted and/or substituted in each of the CDRs (of course, dependent on their respective length), while 1, 2, 3, 4, 5, 6, 7, 8, 9, 10, 11, 12, 13, 14, 15, 16, 17, 18, 19, 20, or 25 amino acids may be inserted, deleted and/or substituted in each of the FRs Amino acid sequence insertions also include N-terminal and/or C-terminal additions of amino acids ranging in length from e.g. 1, 2, 3, 4, 5, 6, 7, 8, 9 or 10 residues to polypeptides containing more than 10, e.g. one hundred or more residues, as well as intra-sequence insertions of single or multiple amino acid residues. An insertional variant of the antibody construct used in accordance with the invention includes the fusion of a polypeptide which increases or extends the serum half-life of the antibody construct to the N-terminus or to the C-terminus of the antibody construct. It is also conceivable that such insertion occurs within the antibody construct, e.g. between the first and the second domain

The sites of greatest interest for amino acid modifications, particularly for amino acid substitutions, include the hypervariable regions, particularly the individual CDRs of the heavy and/or light chain, but FR alterations in the heavy and/or light chain are also contemplated. The substitutions can be conservative substitutions as described herein. Preferably, 1, 2, 3, 4, 5, 6, 7, 8, 9, or 10 amino acids may be substituted in a CDR, while 1, 2, 3, 4, 5, 6, 7, 8, 9, 10, 11, 12, 13, 14, 15, 16, 17, 18, 19, 20, or 25 amino acids may be substituted in the framework regions (FRs), depending on the length of the CDR or FR, respectively. For example, if a CDR sequence encompasses 6 amino acids, it is envisaged that one, two or three of these amino acids are substituted. Similarly, if a CDR sequence encompasses 15 amino acids it is envisaged that one, two, three, four, five or six of these amino acids are substituted.

A useful method for the identification of certain residues or regions within the antibody constructs that are preferred locations for mutagenesis is called “alanine scanning mutagenesis” and is described e.g. in Cunningham B. C. and Wells J. A. (Science. 1989 Jun. 2; 244(4908):1081-5). Here, a residue or group of residues within the antibody construct is/are identified (e.g. charged residues such as Arg, His, Lys, Asp, and Glu) and replaced by a neutral or non-polar amino acid (most preferably alanine or polyalanine) to affect the interaction of the respective amino acid(s) with the epitope of the target protein. Alanine scanning is a technique used to determine the contribution of a specific residue to the stability or function of given protein. Alanine is used because of its non-bulky, chemically inert, methyl functional group that nevertheless mimics the secondary structure preferences that many of the other amino acids possess. Sometimes bulky amino acids such as valine or leucine can be used in cases where conservation of the size of mutated residues is needed. This technique can also be useful to determine whether the side chain of a specific residue plays a significant role in bioactivity. Alanine scanning is usually accomplished by site-directed mutagenesis or randomly by creating a PCR library. Furthermore, computational methods to estimate thermodynamic parameters based on theoretical alanine substitutions have been developed. The data can be tested by IR, NMR Spectroscopy, mathematical methods, bioassays, etc.

Those amino acid locations demonstrating functional sensitivity to the substitutions (as determined e.g. by alanine scanning) can then be refined by introducing further or other variants at, or for, the sites of substitution. Thus, while the site or region for introducing an amino acid sequence variation is predetermined, the nature of the mutation per se needs not to be predetermined. For example, to analyze or optimize the performance of a mutation at a given site, alanine scanning, or random mutagenesis may be conducted at a target codon or region, and the expressed antibody construct variants are screened for the optimal combination of desired activity. Techniques for making substitution mutations at predetermined sites in the DNA having a known sequence are well known, for example, M13 primer mutagenesis and PCR mutagenesis. Screening of the mutants is done e.g. using assays of antigen-binding activity and/or of cytotoxic activity.

Generally, if amino acids are substituted in one or more or all the CDRs of the heavy and/or light chain, it is envisaged that the then-obtained “substituted” sequence is at least 60% or 65%, more preferably 70% or 75%, even more preferably 80% or 85%, and particularly preferably 90% or 95% identical/homologous to the “original” or “parental” CDR sequence. This means that the degree of identity/homology between the original and the substituted sequence depends on the length of the CDR. For example, a CDR having 5 amino acids in total and comprising one amino acid substitution is 80% identical to the “original” or “parental” CDR sequence, while a CDR having 10 amino acids in total and comprising one amino acid substitution is 90% identical to the “original” or “parental” CDR sequence. Accordingly, the substituted CDRs of the antibody construct used in accordance with the invention may have different degrees of identity to their original sequences, e.g., CDRL1 may have 80%, while CDRL3 may have 90% of homology. The same considerations apply to the framework regions and to the entire VH and VL regions.

A “variant CDR” is a CDR with a specific sequence homology, similarity, or identity to the parent CDR used in accordance with the invention, and shares biological function with the parent CDR, including, but not limited to, at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR. Generally, the amino acid homology, similarity, or identity between individual variant CDRs is at least 60% to the parent sequences depicted herein, and more typically with increasing homologies, similarities or identities of at least 65% or 70%, preferably at least 75% or 80%, more preferably at least 85%, 90%, 91%, 92%, 93%, 94%, and most preferably 95%, 96%, 97%, 98%, 99%, and almost 100%. The same applies to “variant VH” and “variant VL”. According to one embodiment, the sequence variations within a “variant VH” and/or a “variant VL” do not extend to the CDRs. The present invention is hence directed to an antibody construct used in accordance with the disclosure herein, comprising VH and VL sequences having a certain sequence homology (see above) to the specific sequences as defined herein (the “parental” VH and VL), wherein the CDR sequences are 100% identical to the specific CDR sequences as defined herein (the “parental” CDRs).

Preferred substitutions (or replacements) are conservative substitutions. However, any substitution (including non-conservative substitutions or one or more from the “exemplary substitutions” listed in Table 1, below) is envisaged, as long as the antibody construct retains its capacity to bind to the target antigen(s) via the first domain and to CD3 or CD3 epsilon via the second domain, and/or provided its CDRs, FRs, VH and/or VL sequences have a degree of identity to the original or parental sequence of at least 60% or 65%, more preferably at least 70% or 75%, even more preferably at least 80% or 85%, and particularly preferably at least 90% or 95%.

A conservative replacement (also called a conservative mutation or a conservative substitution) is an amino acid replacement that changes a given amino acid to a different amino acid with similar biochemical properties (e.g. charge, hydrophobicity, size). Conservative replacements in proteins often have a smaller effect on protein function than non-conservative replacements. Conservative substitutions are shown in Table 1. Exemplary conservative substitutions are shown as “exemplary substitutions”. If such substitutions result in a change in biological activity, then more substantial changes, as further described herein with reference to amino acid classes, may be introduced and the products screened for a desired characteristic.

TABLE 1
Amino acid substitutions (aa = amino acid)

| | Conservative | Exemplary |
|---|---|---|
| Original aa | substitutions | Substitutions |
| Ala (A) | Small aa | Gly, Ser, Thr |
| Arg (R) | Polar aa, particularly Lys | Lys, Gln, Asn |
| Asn (N) | Polar aa, particularly Asp | Asp, Gln, His, |
| | | Lys, Arg |
| Asp (D) | Glu or other polar aa, | Glu, Asn |
| | particularly Asn | |
| Cys (C) | Small aa | Ser, Ala |
| Gln (Q) | Polar aa, particularly Glu | Glu, Asn |
| Glu (E) | Asp or other polar aa, | Asp, Gln |
| | particularly Gln | |
| Gly(G) | Small aa, such as Ala | Ala |
| His (H) | | Asn, Gln, Arg, |
| | | Lys, Tyr |
| Ile (I) | Hydrophobic, particularly | Ala, Val, Met, |
| | aliphatic aa | Leu, Phe |
| Leu (L) | Hydrophobic, particularly | Norleucine, Ile, |
| | aliphatic aa | Ala, Val, Met |
| Lys (K) | Polar aa, particularly Arg | Arg, Gln, Asn |
| Met (M) | Hydrophobic, particularly | Leu, Ala, Ile, |
| | aliphatic aa | Val, Phe |
| Phe (F) | Aromatic or hydrophobic aa, | Tyr, Trp, Leu, |
| | particularly Tyr | Val, Ile, Ala |
| Pro (P) | Small aa | Ala |
| Ser (S) | Polar or small aa, | Thr |
| | particularly Thr | |
| Thr (T) | Polar aa, particularly Ser | Ser |
| Trp (W) | Aromatic aa | Tyr, Phe |
| Tyr (Y) | Aromatic aa, particularly | Phe, Trp, Thr, |
| | Phe | Ser |
| Val (V) | Hydrophobic, particularly | Leu, Ile, Ala, |
| | aliphatic aa | Met, Phe |

Substantial modifications in the biological properties of the antibody construct used in accordance with the present invention are accomplished by selecting substitutions that differ significantly in their effect on maintaining (a) the structure of the polypeptide backbone in the region of the substitution, for example, as a sheet or helical conformation, (b) the charge or hydrophobicity of the molecule at the target site, or (c) the bulk of the side chain. Non-conservative substitutions will usually entail exchanging a member of one of the above defined amino acid classes (such as polar, neutral, acidic, basic, aliphatic, aromatic, small) for another class. Any cysteine residue not involved in maintaining the proper conformation of the antibody construct may be substituted, generally with serine, to improve the oxidative stability of the antibody construct.

Sequence identity, homology and/or similarity of amino acid sequences is determined by using standard techniques known in the art, including, but not limited to, the local sequence identity algorithm of Smith and Waterman, 1981, Adv. Appl. Math. 2:482, the sequence identity alignment algorithm of Needleman and Wunsch (J Mol Biol. 1970 March; 48(3):443-53), the search for similarity method of Pearson and Lipman (Proc Natl Acad Sci USA. 1988 April; 85(8):2444-8), computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al. (Nucleic Acids Res. 1984 Jan. 11; 12(1 Pt 1):387-95), preferably using the default settings, or by inspection. It is envisaged that percent identity is calculated by FastDB based upon the following parameters: mismatch penalty of 1; gap penalty of 1; gap size penalty of 0.33; and joining penalty of 30. See also “Current Methods in Sequence Comparison and Analysis,” Macromolecule Sequencing and Synthesis, Selected Methods and Applications, pp 127-149 (1988), Alan R. Liss, Inc.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng and Doolittle (J Mol Evol. 1987; 25(4):351-60); the method is similar to that described by Higgins and Sharp (Comput Appl Biosci. 1989 April; 5(2):151-3). Useful PILEUP parameters include a default gap weight of 3.00, a default gap length weight of 0.10, and weighted end gaps.

Another example of a useful algorithm is the BLAST algorithm, described in: Altschul et al. (J Mol Biol. 1990 Oct. 5; 215(3):403-10.); Altschul et al., (Nucleic Acids Res. 1997 Sep. 1; 25(17):3389-402); and Karlin and Altschul (Proc Natl Acad Sci USA. 1993 Jun. 15; 90(12):5873-7). A particularly useful BLAST program is the WU-Blast-2 program which was obtained from Altschul et al., (Methods Enzymol. 1996; 266:460-80). WU-Blast-2 uses several search parameters, most of which are set to the default values. The adjustable parameters are set with the following values: overlap span=1, overlap fraction=0.125, word threshold (T)=II. The HSP S and HSP S2 parameters are dynamic values and are established by the program itself depending upon the composition of the specific sequence and composition of the respective database against which the sequence of interest is being searched; however, the values may be adjusted to increase sensitivity.

An additional useful algorithm is gapped BLAST as reported by Altschul et al. (Nucleic Acids Res. 1997 Sep. 1; 25(17):3389-402). Gapped BLAST uses BLOSUM-62 substitution scores; threshold T parameter set to 9; the two-hit method to trigger ungapped extensions, charges gap lengths of k a cost of 10+k; Xu set to 16, and Xg set to 40 for database search stage and to 67 for the output stage of the algorithms. Gapped alignments are triggered by a score corresponding to about 22 bits.

In line herewith, the term “percent (%) nucleic acid sequence identity/homology/similarity” with respect to the nucleic acid sequence encoding the antibody constructs identified herein is defined as the percentage of nucleotide residues in a candidate sequence that are identical with the nucleotide residues in the coding sequence of the antibody construct. One method to align two sequences and thereby determine their homology uses the BLASTN module of WU-Blast2 set to the default parameters, with overlap span and overlap fraction set to 1 and 0.125, respectively. Generally, the nucleic acid sequence homology, similarity, or identity between the nucleotide sequences encoding individual variant CDRs and the nucleotide sequences depicted herein are at least 60%, and more typically with increasing homologies, similarities or identities of at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%, and almost 100%. Again, the same applies to nucleic acid sequence encoding the “variant VH” and/or “variant VL”.

In one embodiment, the percentage of identity to human germline of the antibody constructs used in accordance with the invention, or of the first and second domain (binding domains) of these antibody constructs, is ≥70% or ≥75%, more preferably ≥80% or ≥85%, even more preferably ≥90%, and most preferably ≥91%, ≥92%, ≥93%, ≥94%, ≥95% or even ≥96%. Identity to human antibody germline gene products is thought to be an important feature to reduce the risk of therapeutic proteins to elicit an immune response against the drug in the patient during treatment. Hwang W. Y. and Foote J. (Methods. 2005 May; 36(1):3-10) demonstrate that the reduction of non-human portions of drug antibody constructs leads to a decrease of risk of inducing anti-drug antibodies in the patients during treatment. By comparing an exhaustive number of clinically evaluated antibody drugs and the respective immunogenicity data, the trend is shown that humanization of the variable regions of antibodies/antibody constructs makes the protein less immunogenic (average 5.1% of patients) than antibodies/antibody constructs carrying unaltered non-human variable regions (average 23.59% of patients). Higher degrees of identity to human sequences are hence desirable for protein therapeutics based on variable regions and in the form of antibody constructs. To determine the germline identity, the V-regions of VL can be aligned with the amino acid sequences of human germline V segments and J segments (http://www2.mrc-lmb.cam.ac.uk/vbase/) using Vector NTI software and the amino acid sequence calculated by dividing the identical amino acid residues by the total number of amino acid residues of the VL in percent. The same can be done for the VH segments (http://www2.mrc-lmb.cam.ac.uk/vbase/) with the exception that the VH CDR3 may be excluded due to its high diversity and a lack of existing human germline VH CDR3 alignment partners. Recombinant techniques can then be used to increase sequence identity to human antibody germline genes.

In a further embodiment, the antibody constructs used in accordance with the present invention exhibit high monomer yields under standard research scale conditions, e.g., in a standard two-step purification process. It is envisaged that the monomer yield of the antibody constructs used in accordance with the invention is ≥0.25 mg/L supernatant (SN), preferably ≥0.5 mg/L SN, more preferably ≥1 mg/L SN, even more preferably ≥2 mg/L SN and most preferably ≥3 mg/L SN. The yield of the antibody construct denominated “CL-1×I2C-6His” was shown to be 4.1 mg/L supernatant, and the yield of the antibody construct denominated “CL-1×I2C-scFc” was shown to be 36.5 mg/L supernatant.

Likewise, the yield of the dimeric antibody construct isoforms and hence the monomer percentage (i.e., monomer:(monomer+dimer)) of the antibody constructs can be determined. The productivity of monomeric and dimeric antibody constructs and the calculated monomer percentage can e.g. be obtained in the SEC purification step of culture supernatant from standardized research-scale production in roller bottles. According to one embodiment, the monomer percentage of the antibody constructs used in accordance with the invention is ≥80%, more preferably ≥85%, even more preferably ≥90%, and most preferably ≥95%.

According to one embodiment, the antibody constructs used in accordance with the invention have a plasma stability (ratio of EC₅₀ with plasma to EC₅₀ w/o plasma) of ≤5 or ≤4, more preferably ≤3.5 or ≤3, even more preferably ≤2.5 or ≤2, and most preferably ≤1.5 or ≤1. The plasma stability of an antibody construct can be tested by incubation of the purified construct in human plasma at 37° C. for 24 to 96 hours, e.g. at a concentration of 2-20 μg/ml, followed by EC₅₀ determination in an 18 h 51-chromium release or in a 48 h FACS cytotoxicity assay. The effector cells in the cytotoxicity assay can be stimulated enriched human CD8 positive T cells (preferred) or unstimulated human PBMC. Target cells can e.g. be CHO cells transfected with human the target antigen(s). The effector to target cell (E:T) ratio can be 10:1. The starting concentration of the antibody constructs in the cytotoxicity assay can be 0.01-0.1 μg/ml. The human plasma pool used for this purpose is derived from the blood of healthy donors collected by EDTA coated syringes. Cellular components are removed by centrifugation and the upper plasma phase is collected and subsequently pooled. As control, non-incubated antibody constructs are diluted immediately prior to the cytotoxicity assay in appropriate medium such as RPMI-1640. The plasma stability is calculated as ratio of EC₅₀ (after plasma incubation) to EC₅₀ (control/no incubation).

It is furthermore envisaged that the monomer to dimer conversion of the antibody constructs used in accordance with the invention is low. The conversion can be measured under different conditions and analyzed by high performance size exclusion chromatography. For example, incubation of the monomeric isoforms of the antibody constructs can be carried out for 7 days at 37° C. in generic formulation buffer and at concentrations of e.g. 100 μg/ml or 250 μg/ml in an incubator, followed by high performance SEC to determine the percentage of initially monomeric antibody construct which had been converted into dimeric antibody construct. Under these conditions, it is envisaged that the antibody constructs used in accordance with the invention show a dimer percentage that is ≤8%, preferably ≤6%, more preferably ≤5%, more preferably ≤4%, even more preferably ≤3%, even more preferably ≤2.5%, even more preferably ≤2%, even more preferably ≤1.5%, and most preferably ≤1% or ≤0.5% or even 0%.

It is likewise envisaged that the antibody constructs used in accordance with the present invention present with very low dimer conversion after several freeze/thaw cycles. For example, the antibody construct monomer is adjusted to a concentration of 250 μg/ml e.g. in generic formulation buffer and subjected to three freeze/thaw cycles (freezing at −80° C. for 30 min followed by thawing for 30 min at room temperature), followed by high performance SEC to determine the percentage of initially monomeric antibody construct which had been converted into dimeric antibody construct. It is envisaged that the dimer percentages of the antibody constructs are ≤8%, preferably ≤6%, more preferably ≤5%, more preferably ≤4%, even more preferably ≤3%, even more preferably ≤2.5%, even more preferably ≤2%, even more preferably ≤1.5%, and most preferably ≤1% or ≤0.5% or even 0%, for example after three freeze/thaw cycles.

According to one embodiment, the antibody constructs used in accordance with the present invention show a favorable thermostability with aggregation temperatures ≥45° C. or ≥46° C., more preferably ≥47° C. or ≥48° C., even more preferably ≥49° C. or ≥50° C., and most preferably ≥51° C. The thermostability parameter can be determined in terms of antibody aggregation temperature as follows: Antibody solution at a concentration 250 μg/ml is transferred into a single use cuvette and placed in a dynamic light scattering (DLS) device. The sample is heated from 40° C. to 70° C. at a heating rate of 0.5° C./min with constant acquisition of the measured radius. Increase of radius indicating melting of the protein and aggregation is used to calculate the aggregation temperature of the antibody.

Alternatively, temperature melting curves can be determined by differential scanning calorimetry (DSC) to determine intrinsic biophysical protein stabilities of the antibody constructs. These experiments can be performed using a MicroCal LLC VP-DSC device. The energy uptake of a sample containing an antibody construct is recorded from 20° C. to 90° C. compared to a sample containing only the formulation buffer. The antibody constructs are adjusted to a final concentration of 250 μg/ml e.g. in SEC running buffer. For recording of the respective melting curve, the overall sample temperature is increased stepwise. Energy uptake of the sample and the formulation buffer reference is recorded at each temperature. The difference in energy uptake Cp (kcal/mole/° C.) of the sample minus the reference is plotted against the respective temperature. The melting temperature is defined as the temperature at the first maximum of energy uptake.

The antibody constructs used in accordance with the invention are also envisaged to have a turbidity of ≤0.2 or ≤0.15, preferably of ≤0.10 or ≤0.08, more preferably of ≤0.06 or ≤0.05, and most preferably of ≤0.04 or ≤0.03. The turbidity can be measured by OD340 at a concentration of the antibody construct of 2.5 mg/ml and 16 h incubation at 5° C.

Changes in the potency of a target×CD3 antibody construct as a function of preincubation of the construct on the target cells in the absence of T cells can be measured. If an antibody construct is internalized, it is expected to undergo lysosomal degradation. The effective concentration is hence expected to decrease over time, and thus the apparent potency should decrease as well. The effect has been observed with some targets, for which this is a known phenomenon. Antibody constructs used in accordance with the invention are envisaged to not be internalized or to not undergo significant internalization by the target cell. The rate of internalization can be assayed e.g. as described in the following: T cells are counted and diluted to a concentration of 1×10⁵/ml in assay media. Target positive target cells are counted and plated e.g. at 2500 cells per well (cpw). The antibody construct is diluted serially 1:2, e.g. at a starting concentration of 100 nM. The antibody construct is added to the culture assay plates to allow for 0 hours, 1 hour or 2 hours of incubation prior to addition of the T cells. Then the T cells are plated at 25000 cpw (E:T=10:1), and the assay is incubated for 48 hours at 37° C. Target cell survival is analyzed e.g. with the Steady-Glo® system (25 μl/well). Preferably, the internalization rate (e.g. measured as a decrease in cytotoxicity) is ≤20% after a 2-hour (pre-)incubation of the antibody construct with the target cell, more preferably ≤15%, even more preferably ≤10%, and most preferably ≤5%.

It is furthermore envisaged for an antibody construct used in accordance with the invention that shed or soluble target does not significantly impair its efficacy or biologic activity. This can be measured e.g. in a cytotoxicity assay where soluble target is added at increasing concentrations to the assay, e.g. at 0 nM-0.3 nM-0.7 nM-1 nM-3 nM-7 nM-12 nM. An exemplary E:T value is 10:1. The EC50 value of the tested antibody construct should not be significantly increased in the presence of soluble target.

In a further embodiment, the antibody construct used in accordance with the invention is stable at acidic pH. The more tolerant the antibody construct behaves at unphysiologic pH such as pH 5.5 (a pH which is required to run e.g. a cation exchange chromatography), the higher is the recovery of the antibody construct eluted from an ion exchange column relative to the total amount of loaded protein. Recovery of the antibody construct from an ion (e.g., cation) exchange column at pH 5.5 is preferably ≥30%, more preferably ≥40%, more preferably ≥50%, even more preferably ≥60%, even more preferably ≥70%, even more preferably ≥80%, and most preferably ≥95%. The percentage represents the area under the curve (=AUC) of the main peak.

It is furthermore envisaged that the antibody constructs used in accordance with the present invention exhibit therapeutic efficacy, which manifests as anti-tumor activity or tumor growth inhibition. In one embodiment, the tumor growth inhibition of the antibody construct used in accordance with the invention T/C [%] is ≤70, ≤60, ≤50, ≤40, ≤30, ≤20, ≤10, ≤5, ≤4, ≤3, or ≤2. Modification or adjustment of certain parameters of these studies (such as the number of injected tumor cells, the site of injection, the number of transplanted human T cells, the amount of antibody constructs to be administered, and the timelines) is also envisaged, while still arriving at a meaningful and reproducible result.

The invention further provides a polynucleotide/nucleic acid molecule encoding an antibody construct used in accordance with the invention. Nucleic acid molecules are biopolymers composed of nucleotides. A polynucleotide is a biopolymer composed of 13 or more nucleotide monomers covalently bonded in a chain. DNA (such as cDNA) and RNA (such as mRNA) are examples of polynucleotides/nucleic acid molecules with distinct biological function. Nucleotides are organic molecules that serve as the monomers or subunits of nucleic acid molecules like DNA or RNA. The nucleic acid molecule or polynucleotide can be double stranded or single stranded, linear or circular. It is envisaged that the nucleic acid molecule or polynucleotide is comprised in a vector. It is furthermore envisaged that such vector is comprised in a host cell. Said host cell is, e.g. after transformation or transfection with the vector or the polynucleotide/nucleic acid molecule, capable of expressing the antibody construct. For this purpose, the polynucleotide or nucleic acid molecule is operatively linked with control sequences.

The genetic code is the set of rules by which information encoded within genetic material (nucleic acids) is translated into proteins. Biological decoding in living cells is accomplished by the ribosome which links amino acids in an order specified by mRNA, using tRNA molecules to carry amino acids and to read the mRNA three nucleotides at a time. The code defines how sequences of these nucleotide triplets, called codons, specify which amino acid will be added next during protein synthesis. With some exceptions, a three-nucleotide codon in a nucleic acid sequence specifies a single amino acid. Because the majority of genes are encoded with exactly the same code, this particular code is often referred to as the canonical or standard genetic code.

Degeneracy of codons is the redundancy of the genetic code, exhibited as the multiplicity of three-base pair codon combinations that specify an amino acid. Degeneracy results because there are more codons than encodable amino acids. The codons encoding one amino acid may differ in any of their three positions; however, often this difference is in the second or third position. For instance, codons GAA and GAG both specify glutamic acid and exhibit redundancy; but, neither specifies any other amino acid and thus demonstrate no ambiguity. The genetic codes of different organisms can be biased towards using one of the several codons that encode the same amino acid over the others—that is, a greater frequency of one will be found than expected by chance. For example, leucine is specified by six distinct codons, some of which are rarely used. Codon usage tables detailing genomic codon usage frequencies for most organisms are available. Recombinant gene technologies commonly take advantage of this effect by implementing a technique termed codon optimization, in which those codons are used to design a polynucleotide which are preferred by the respective host cell (such as a cell of human hamster origin, an Escherichia coli cell, or a Saccharomyces cerevisiae cell), e.g. to increase protein expression. It is hence envisaged that the polynucleotides/nucleic acid molecules of the present disclosure are codon optimized. Nevertheless, the polynucleotide/nucleic acid molecule encoding an antibody construct used in accordance with the invention may be designed using any codon that encodes the desired amino acid.

According to one embodiment, the polynucleotide/nucleic acid molecule encoding the antibody construct used in accordance with the invention is in the form of one single molecule or in the form of two or more separate molecules. If the antibody construct used in accordance with the present invention is a single chain antibody construct, the polynucleotide/nucleic acid molecule encoding such construct will most likely also be in the form of one single molecule. However, it is also envisaged that different components of the antibody construct (such as the different domains, e.g. the domain which binds to the target antigen(s), the domain which binds to CD3, and/or further domains such as antibody constant domains) are located on separate polypeptide chains, in which case the polynucleotide/nucleic acid molecule is most likely in the form of two or more separate molecules.

The same applies for the vector comprising a polynucleotide/nucleic acid molecule. If the antibody construct used in accordance with the present invention is a single chain antibody construct, one vector may comprise the polynucleotide which encodes the antibody construct in one single location (as one single open reading frame, ORF). One vector may also comprise two or more polynucleotides/nucleic acid molecules at separate locations (with individual ORFs), each one of them encoding a different component of the antibody construct used in accordance with the invention. It is envisaged that the vector comprising the polynucleotide/nucleic acid molecule of the present invention is in the form of one single vector or two or more separate vectors. In one embodiment, and for the purpose of expressing the antibody construct in a host cell, the host cell should comprise the polynucleotide/nucleic acid molecule encoding the antibody construct or the vector comprising such polynucleotide/nucleic acid molecule in their entirety, meaning that all components of the antibody construct—whether encoded as one single molecule or in separate molecules/locations—will assemble after translation and form together the biologically active antibody construct used in accordance with the invention.

Also disclosed herein is a vector comprising a polynucleotide/nucleic acid molecule used in accordance with the invention. A vector is a nucleic acid molecule used as a vehicle to transfer (foreign) genetic material into a cell, usually to ensure the replication and/or expression of the genetic material. The term “vector” encompasses—but is not restricted to—plasmids, viruses, cosmids, and artificial chromosomes. Some vectors are designed specifically for cloning (cloning vectors), others for protein expression (expression vectors). So-called transcription vectors are mainly used to amplify their insert. The manipulation of DNA is normally conducted on E. coli vectors, which contain elements necessary for their maintenance in E. coli. However, vectors may also have elements that allow them to be maintained in another organism such as yeast, plant or mammalian cells, and these vectors are called shuttle vectors. Insertion of a vector into the target or host cell is usually called transformation for bacterial cells and transfection for eukaryotic cells, while insertion of a viral vector is often called transduction.

In general, engineered vectors comprise an origin of replication, a multicloning site and a selectable marker. The vector itself is generally a nucleotide sequence, commonly a DNA sequence, that comprises an insert (transgene) and a larger sequence that serves as the “backbone” of the vector. While the genetic code determines the polypeptide sequence for a given coding region, other genomic regions can influence when and where these polypeptides are produced. Modern vectors may therefore encompass additional features besides the transgene insert and a backbone: promoter, genetic marker, antibiotic resistance, reporter gene, targeting sequence, protein purification tag. Vectors called expression vectors (expression constructs) specifically are for the expression of the transgene in the target cell, and generally have control sequences.

The term “control sequences” refers to DNA sequences necessary for the expression of an operably linked coding sequence in a specific host organism. The control sequences that are suitable for prokaryotes, for example, include a promoter, optionally an operator sequence, and a ribosome binding site. Eukaryotic cells are known to utilize promoters, polyadenylation signals, a Kozak sequence and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned to facilitate translation. Generally, “operably linked” means that the nucleotide sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Transfection” is the process of deliberately introducing nucleic acid molecules or polynucleotides (including vectors) into target cells. The term is mostly used for non-viral methods in eukaryotic cells. Transduction is often used to describe virus-mediated transfer of nucleic acid molecules or polynucleotides. Transfection of animal cells typically involves opening transient pores or “holes” in the cell membrane, to allow the uptake of material. Transfection can be carried out using biological particles (such as viral transfection, also called viral transduction), chemical-based methods (such as using calcium phosphate, lipofection, Fugene, cationic polymers, nanoparticles) or physical treatment (such as electroporation, microinjection, gene gun, cell squeezing, magnetofection, hydrostatic pressure, impalefection, sonication, optical transfection, heat shock).

The term “transformation” is used to describe non-viral transfer of nucleic acid molecules or polynucleotides (including vectors) into bacteria, and into non-animal eukaryotic cells, including plant cells. Transformation is hence the genetic alteration of a bacterial or non-animal eukaryotic cell resulting from the direct uptake through the cell membrane(s) from its surroundings and subsequent incorporation of exogenous genetic material (nucleic acid molecules). Transformation can be achieved by artificial means. For transformation to happen, cells or bacteria must be in a state of competence, which might occur as a time-limited response to environmental conditions such as starvation and cell density and can also be artificially induced.

Moreover, disclosed is a host cell transformed or transfected with the polynucleotide/nucleic acid molecule used in accordance with the invention or with the vector used in accordance with the invention.

As used herein, the terms “host cell” or “recipient cell” are intended to include any individual cell or cell culture that can be or has been recipient of vectors, exogenous nucleic acid molecules and/or polynucleotides encoding the antibody construct used in accordance with the present invention; and/or recipients of the antibody construct itself. The introduction of the respective material into the cell is carried out by way of transformation, transfection and the like (vide supra). The term “host cell” is also intended to include progeny or potential progeny of a single cell. Because certain modifications may occur in succeeding generations due to either natural, accidental, or deliberate mutation or due to environmental influences, such progeny may not, in fact, be completely identical (in morphology or in genomic or total DNA complement) to the parent cell but is still included within the scope of the term as used herein. Suitable host cells include prokaryotic or eukaryotic cells and include—but are not limited to—bacteria (such as E. coli), yeast cells, fungi cells, plant cells, and animal cells such as insect cells and mammalian cells, e.g., hamster, murine, rat, macaque or human

In addition to prokaryotes, eukaryotic microbes such as filamentous fungi or yeast are suitable cloning or expression hosts for the antibody construct used in accordance with the invention. Saccharomyces cerevisiae, or common baker's yeast, is the most commonly used among lower eukaryotic host microorganisms. However, a number of other genera, species, and strains are commonly available and useful herein, such as Schizosaccharomyces pombe, Kluyveromyces hosts such as K. lactis, K fragilis (ATCC 12424), K. bulgaricus (ATCC 16045), K. wickeramii (ATCC 24178), K. waltii (ATCC 56500), K. drosophilarum (ATCC 36906), K. thermotolerans, and K. marxianus; Yarrowia (EP 402 226); Pichia pastoris (EP 183 070); Candida; Trichoderma reesia (EP 244 234); Neurospora crassa; Schwanniomyces such as Schwanniomyces occidentalis; and filamentous fungi such as Neurospora, Penicillium, Tolypocladium, and Aspergillus hosts such as A. nidulans and A. niger.

Suitable host cells for the expression of a glycosylated antibody construct are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruit fly), and Bombyx mori (silkmoth) have been identified. A variety of viral strains for transfection are publicly available, e.g., the L-1 variant of Autographa californica NPV and the Bm-5 strain of Bombyx mori NPV, and such viruses may be used as the virus, particularly for transfection of Spodoptera frugiperda cells.

Plant cell cultures of cotton, corn, potato, soybean, petunia, tomato, Arabidopsis and tobacco can also be used as hosts. Cloning and expression vectors useful in the production of proteins in plant cell culture are known to those of skill in the art. See e.g. Hiatt et al., Nature (1989) 342: 76-78, Owen et al. (1992) Bio/Technology 10: 790-794, Artsaenko et al. (1995) The Plant J 8: 745-750, and Fecker et al. (1996) Plant Mol Biol 32: 979-986.

However, interest has been greatest in vertebrate cells, and propagation of vertebrate cells in culture (cell culture) has become a routine procedure. Examples of useful mammalian host cell lines are monkey kidney CV1 line transformed by SV40 (such as COS-7, ATCC CRL 1651); human embryonic kidney line (such as 293 or 293 cells subcloned for growth in suspension culture, Graham et al., J. Gen Virol. 36: 59 (1977)); baby hamster kidney cells (such as BHK, ATCC CCL 10); Chinese hamster ovary cells/-DHFR (such as CHO, Urlaub et al., Proc. Natl. Acad. Sci. USA 77: 4216 (1980)); mouse sertoli cells (such as TM4, Mather, Biol. Reprod. 23: 243-251 (1980)); monkey kidney cells (such as CVI ATCC CCL 70); African green monkey kidney cells (such as VERO-76, ATCC CRL1587); human cervical carcinoma cells (such as HELA, ATCC CCL 2); canine kidney cells (such as MDCK, ATCC CCL 34); buffalo rat liver cells (such as BRL 3A, ATCC CRL 1442); human lung cells (such as W138, ATCC CCL 75); human liver cells (such as Hep G2,1413 8065); mouse mammary tumor (such as MMT 060562, ATCC CCL-51); TRI cells (Mather et al., Annals N. Y Acad. Sci. (1982) 383: 44-68); MRC 5 cells; FS4 cells; and a human hepatoma line (such as Hep G2).

Also disclosed herein is a process for producing an antibody construct used in accordance with the invention, said process comprising culturing a host cell n under conditions allowing the expression of the antibody construct used in accordance with the invention and recovering the produced antibody construct from the culture.

As used herein, the term “culturing” refers to the in vitro maintenance, differentiation, growth, proliferation and/or propagation of cells under suitable conditions in a medium. Cells are grown and maintained in a cell growth medium at an appropriate temperature and gas mixture. Culture conditions vary widely for each cell type. Typical growth conditions are a temperature of about 37° C., a CO2 concentration of about 5% and a humidity of about 95%. Recipes for growth media can vary e.g. in pH, concentration of the carbon source (such as glucose), nature and concentration of growth factors, and the presence of other nutrients (such as amino acids or vitamins). The growth factors used to supplement media are often derived from the serum of animal blood, such as fetal bovine serum (FBS), bovine calf serum (FCS), equine serum, and porcine serum. Cells can be grown either in suspension or as adherent cultures. There are also cell lines that have been modified to be able to survive in suspension cultures, so they can be grown to a higher density than adherent conditions would allow.

The term “expression” includes any step involved in the production of an antibody construct used in accordance with the invention including, but not limited to, transcription, post-transcriptional modification, translation, folding, post-translational modification, targeting to specific subcellular or extracellular locations, and secretion. The term “recovering” refers to a series of processes intended to isolate the antibody construct from the cell culture. The “recovering” or “purification” process may separate the protein and non-protein parts of the cell culture, and finally separate the desired antibody construct from all other polypeptides and proteins. Separation steps usually exploit differences in protein size, physico-chemical properties, binding affinity and biological activity. Preparative purifications aim to produce a relatively large quantity of purified proteins for subsequent use, while analytical purification produces a relatively small amount of a protein for a variety of research or analytical purposes.

When using recombinant techniques, the antibody construct can be produced intracellularly, in the periplasmic space, or directly secreted into the medium. If the antibody construct is produced intracellularly, as a first step, the particulate debris, either host cells or lysed fragments, are removed, for example, by centrifugation or ultrafiltration. The antibody construct used in accordance with the invention may e.g. be produced in bacteria such as E. coli. After expression, the construct is isolated from the bacterial cell paste in a soluble fraction and can be purified e.g. via affinity chromatography and/or size exclusion. Final purification can be carried out in a manner that is similar to the process for purifying an antibody construct expressed in mammalian cells and secreted into the medium. Carter et al. (Biotechnology (NY) 1992 February; 10(2):163-7) describe a procedure for isolating antibodies which are secreted to the periplasmic space of E. coli.

Where the antibody is secreted into the medium, supernatants from such expression systems are generally first concentrated using a commercially available protein concentration filter, for example, an ultrafiltration unit.

The antibody construct used in accordance with the invention prepared from the host cells can be recovered or purified using, for example, hydroxylapatite chromatography, gel electrophoresis, dialysis, and affinity chromatography. Other techniques for protein purification such as fractionation on an ion-exchange column, mixed mode ion exchange, HIC, ethanol precipitation, size exclusion chromatography, reverse phase HPLC, chromatography on silica, chromatography on heparin sepharose, chromatography on an anion or cation exchange resin (such as a polyaspartic acid column), immunoaffinity (such as Protein A/G/L) chromatography, chromato-focusing, SDS-PAGE, ultracentrifugation, and ammonium sulfate precipitation are also available depending on the antibody construct to be recovered.

A protease inhibitor may be included in any of the foregoing steps to inhibit proteolysis, and antibiotics may be included to prevent the growth of contaminants

According to the invention, the combination therapy or combination products/compositions comprise an inhibitor/antagonist of TNF/TNFR. TNF is a cytokine that is involved in inflammation and regulation of the immune system. It is mainly produced by activated macrophages and well-known in the art (Holbrook, J. et al., F1000Research 2019, 8(F1000 Faculty Rev):111). Human TNF binds to two receptors, TNFR1 and TNFR2, respectively. While TNFR1 is expressed on essentially all human tissues, expression of TNFR2 is primarily confined to cells of the immune system, neurons, and endothelial cells. Interaction of TNF with its receptors results in conformational changes and binding to its receptor(s) induces, depending on the type of receptor (TNFR1 or TNFR2) and different signalling cascades, e.g., cell death by apoptosis or necrosis, but also cell proliferation, tissue regeneration and inflammation. Agonists/inhibitors of TNF, e.g., infliximab, etanercept, etc., are used as drugs in the treatment of various diseases such as autoimmune diseases (Rheumatoid Arthritis, Crohn's Disease, Ankylosing Spondylitis, etc.; Sedger and McDermott, Cytokine & Growth Factor Reviews 25 (2014) 453-472). In one particular embodiment of the invention, the antagonist/inhibitor of TNF/TNFR-mediated signalling is etanercept that is used in combination with an antibody construct as defined in any one of the preceding paragraphs.

Moreover, the invention provides a pharmaceutical composition or formulation comprising an antibody construct used in accordance with the invention or an antibody construct produced according to the process disclosed herein in combination with an inhibitor/antagonist of TNF/TNFR.

As used herein, an “inhibitor” or “antagonist” of TNF/TNFR is capable of completely or partially blocking or reducing TNF/TNFR-signalling. In embodiments of the invention, the “inhibitor” or “antagonist” of TNF/TNFR blocks signalling by at least 10%, at least 20%, at least 30%, at least 40%, at least 50%, at least 60%, at least 70%, at least 80%, at least 90%, at least 95%, or completely, i.e. the effects based on TNF/TNFR-signalling that can be measured using suitable assays are no longer or only partially detectable. Suitable assays for the measurement of TNF/TNFR-signalling are, for example, based on the detection of TNFR-induced activation of NF-κB, but any other reliable and art-recognized test in vitro can be used (see also Chapter 4 of Žigon-Branč, Barlič and Jeras' publication in IntechOpen entitled: In vitro Cell-Based Assays for Potency Testing of Anti-TNF-α Biological Drugs; Mar. 25, 2019 and references cited therein). “Reducing” has its commonly accepted meaning, i.e. the amount of a given parameter (number, signalling activity, etc.) is lowered compared to the status without exposure to a herein described inhibitor/antagonist.

Examples of “inhibitors” or “antagonists” of TNF/TNFR signalling are FDA)- and EMA-approved originator and biosimilar anti-TNF drugs, i.e. full-length monoclonal antibodies (mAbs) infliximab (IFX), a chimeric mouse/human mAb (Remicade® and its biosimilars: Remsima®, Inflectra®, Flixabi®, Ixifi®, Renflexis®, and Zessly®), adalimumab (ADA), a fully humanized mAb (Humira® and its biosimilars: Cyltezo®, Imraldi®, Amgevita®, Solymbic®, Hyrimoz®, Hulio®, Halimatoz®, and Heyifa®), and golimumab, another fully humanized mAb (Simponi®). The additional two anti-TNF biological drugs, which are not mAbs, are etanercept (ETA) (Enbrel® and its biosimilars: Erelzi® and Benepali®), a fusion protein consisting of two extracellular parts of the human TNFR2 and the Fc portion of human IgG1, and certolizumab pegol (Cimzia®) composed of a human Fab′ fragment, covalently attached to two cross-linked 20 kDa polyethylene glycol chains.

According to one embodiment of the present invention, the pharmaceutical compositions comprises an antagonist/inhibitor of TNF/TNFR-mediated signalling, which is preferably etanercept in combination with an antibody construct as defined in any one of the preceding paragraphs.

As used herein, the term “pharmaceutical composition” relates to a composition which is suitable for administration to a patient, preferably a human patient. The particularly preferred pharmaceutical composition of this invention comprises one or a plurality of the antibody construct(s) used in accordance with the invention, preferably in a therapeutically effective amount, in combination with an inhibitor/antagonist of TNF/TNFR, preferably in a therapeutically effective amount. Preferably, the pharmaceutical composition further comprises suitable formulations of one or more (pharmaceutically effective) carriers, stabilizers, excipients, diluents, solubilizers, surfactants, emulsifiers, preservatives and/or adjuvants.

Acceptable constituents of the composition are preferably nontoxic to recipients at the dosages and concentrations employed. Pharmaceutical compositions of the invention include, but are not limited to, liquid, frozen, and lyophilized compositions. The pharmaceutical compositions according to the present invention are combination products that may be combined to form a single pharmaceutical composition immediately prior to their administration to a patient in need thereof. Alternatively, the combination products may be prepared as pharmaceutical compostions by the manufacturer and may be ready used. This includes also combination products that must be reconstituted, diluted, or otherwise handled prior to use, but which contain already the active ingredients disclosed herein, i.e. one or a plurality of the antibody construct(s) used in accordance with the invention, preferably in a therapeutically effective amount, in combination with an inhibitor/antagonist of TNF/TNFR, preferably in a therapeutically effective amount.

The compositions may comprise a pharmaceutically acceptable carrier. In general, as used herein, “pharmaceutically acceptable carrier” means all aqueous and non-aqueous solutions, sterile solutions, solvents, buffers, e.g. phosphate buffered saline (PBS) solutions, water, suspensions, emulsions, such as oil/water emulsions, various types of wetting agents, liposomes, dispersion media and coatings, which are compatible with pharmaceutical administration, in particular with parenteral administration. The use of such media and agents in pharmaceutical compositions is well known in the art, and the compositions comprising such carriers can be formulated by well-known conventional methods.

Certain embodiments provide pharmaceutical compositions comprising the antibody construct used in accordance with the invention and further one or more excipients such as those illustratively described in this section and elsewhere herein. Excipients can be used in the invention for a wide variety of purposes, such as adjusting physical, chemical, or biological properties of formulations, such as adjustment of viscosity, and or processes to improve effectiveness and/or to stabilize such formulations and processes against degradation and spoilage e.g. due to stresses that occur during manufacturing, shipping, storage, pre-use preparation, administration, and thereafter. Excipients should in general be used in their lowest effective concentrations.

In certain embodiments, the pharmaceutical composition may contain formulation materials for modifying, maintaining or preserving certain characteristics of the composition such as the pH, osmolarity, viscosity, clarity, color, isotonicity, odor, sterility, stability, rate of dissolution or release, adsorption or penetration (see, Remington's Pharmaceutical Sciences, 18″ Edition, 1990, Mack Publishing Company). In such embodiments, suitable formulation materials may include, but are not limited to:
amino acids
antimicrobials such as antibacterial and antifungal agents
antioxidants
buffers, buffer systems and buffering agents which are used to maintain the composition at physiological pH or at a slightly lower pH, typically within a pH range of from about 5 to about 8 or 9
non-aqueous solvents, vegetable oils, and injectable organic esters
aqueous carriers including water, alcoholic/aqueous solutions, emulsions or suspensions, including saline and buffered media
biodegradable polymers such as polyesters
bulking agents
chelating agents
isotonic and absorption delaying agents
complexing agents
fillers
carbohydrates
(low molecular weight) proteins, polypeptides or proteinaceous carriers, preferably of human origin
coloring and flavouring agents
sulfur containing reducing agents
diluting agents
emulsifying agents
hydrophilic polymers
salt-forming counter-ions
preservatives
metal complexes
solvents and co-solvents
sugars and sugar alcohols
suspending agents
surfactants or wetting agents
stability enhancing agents
tonicity enhancing agents
parenteral delivery vehicles
intravenous delivery vehicles

It is common knowledge that the different constituents of the pharmaceutical composition can have different effects, for example, and amino acid can act as a buffer, a stabilizer and/or an antioxidant; mannitol can act as a bulking agent and/or a tonicity enhancing agent; sodium chloride can act as delivery vehicle and/or tonicity enhancing agent; etc.

In the context of the present invention, a pharmaceutical composition may comprise:

(a) an antibody construct as described herein,
(b) at least one buffer agent,
(c) at least one saccharide, and
(d) at least one surfactant;
wherein the pH of the pharmaceutical composition is in the range of 3.5 to 6.0.

In the composition described above, the first domain preferably has an isoelectric point (pI) in the range of 4 to 9.5; the second domain has a pI in the range of 8 to 10, preferably 8.5 to 9.0; and the antibody construct optionally comprises a third domain comprising two polypeptide monomers, each comprising a hinge, a CH2 domain and a CH3 domain, wherein said two polypeptide monomers are fused to each other via a peptide linker;

In the composition described above, it is further envisaged that the at least one buffer agent is present at a concentration range of 5 to 200 mM, more preferably at a concentration range of 10 to 50 mM. It is also envisaged that the at least one saccharide is selected from the group consisting of monosaccharide, disaccharide, cyclic polysaccharide, sugar alcohol, linear branched dextran or linear non-branched dextran. It is also envisaged that the disaccharide is selected from the group consisting of sucrose, trehalose and mannitol, sorbitol, and combinations thereof. It is further envisaged that the sugar alcohol is sorbitol. It is also envisaged that the at least one saccharide is present at a concentration in the range of 1 to 15% (m/V), preferably in a concentration range of 9 to 12% (m/V). It is further envisaged that the antibody construct is present in a concentration range of 0.1 to 8 mg/ml, preferably of 0.2-2.5 mg/ml, more preferably of 0.25-1.0 mg/ml.

According to one embodiment of the composition described above, the at least one surfactant is selected from the group consisting of polysorbate 20, polysorbate 40, polysorbate 60, polysorbate 80, poloxamer 188, pluronic F68, triton X-100, polyoxyethylen, PEG 3350, PEG 4000 and combinations thereof. It is further envisaged that the at least one surfactant is present at a concentration in the range of 0.004 to 0.5% (m/V), preferably in the range of 0.001 to 0.01% (m/V). It is envisaged that the pH of the composition is in the range of 4.0 to 5.0, preferably 4.2. It is also envisaged that the pharmaceutical composition has an osmolarity in the range of 150 to 500 mOsm. It is further envisaged that the pharmaceutical composition further comprises an excipient selected from the group consisting of one or more polyol(s) and one or more amino acid(s). It is envisaged in the context of the present invention that said one or more excipient is present in the concentration range of 0.1 to 15 (w/V).

The present invention also provides a pharmaceutical composition comprising

(a) the antibody construct as described herein, preferably in a concentration range of 0.1 to 8 mg/ml, preferably of 0.2-2.5 mg/ml, more preferably of 0.25-1.0 mg/ml;
(b) 10 mM glutamate or acetate;
(c) 9% (m/V) sucrose or 6% (m/V) sucrose and 6% (m/V) hydroxypropyl-β-cyclodextrin;
(d) 0.01% (m/V) polysorbate 80;
wherein the pH of the liquid pharmaceutical composition is 4.2.

It is envisaged that the composition of the invention might comprise, in addition to the antibody construct and the inhibitor or antagonist of TNF/TNFR-mediated signalling used in accordance with the invention defined herein, further biologically active agents, depending on the intended use of the composition. Such agents might be drugs acting on the gastro-intestinal system, drugs acting as cytostatica, drugs preventing hyperurikemia, drugs inhibiting immunoreactions, drugs modulating the inflammatory response, drugs acting on the circulatory system and/or agents such as cytokines known in the art. It is also envisaged that the antibody construct used in accordance with the present invention is applied in a co-therapy, i.e., in combination with another anti-cancer medicament.

In this context, it is envisaged that the pharmaceutical composition of the invention (which comprises an antibody construct comprising a first domain which binds to the target antigen(s) on the surface of a target cell and a second domain which binds to CD3 on the surface of a T cell, and an inhibitor/antagonist of TNF/TNFR signalling, as described in more detail herein above) furthermore comprises an agent, preferably an antibody or antibody construct, which binds to a protein of the immune checkpoint pathway (such as PD-1 or CTLA-4) or to a co-stimulatory immune checkpoint receptor (such as 4-1BB). The present invention also refers to a combination of an antibody construct according used in accordance with the invention (which comprises an antibody construct comprising a first domain which binds to the target antigen(s) on the surface of a target cell and a second domain which binds to CD3 on the surface of a T cell, and an inhibitor/antagonist of TNF/TNFR signalling, as described in more detail herein above) and an agent, preferably an antibody or antibody construct, which binds to a protein of the immune checkpoint pathway (such as PD-1 or CTLA-4) or to a co-stimulatory immune checkpoint receptor (such as 4-1BB). Due to the nature of the at least three ingredients of the combination, namely their pharmaceutical activity, the combination can also be referred to as a therapeutic combination. In some embodiments, the combination can be in the form of a pharmaceutical composition or of a kit. According to one embodiment, the pharmaceutical composition or the combination comprises an antibody construct used in accordance with the invention, an inhibitor/antagonist of TNF/TNFR signalling, and an antibody or antibody construct which binds to PD-1. Anti-PD-1 binding proteins useful for this purpose are e.g. described in detail in PCT/US2019/013205.

Hence, in a further aspect, the present invention is directed to a pharmaceutical composition or to a combination comprising:
an antibody construct comprising a first domain which binds to the target antigen(s) on the surface of a target cell and a second domain which binds to CD3 on the surface of a T cell and at least one inhibitor/antagonist of TNF/TNFR, as described in more detail herein above, and
an antibody or antibody construct which binds to PD-1 and comprises:
   a) a VH region comprising CDR-H1 as depicted in SEQ ID NO: 122, CDR-H2 as depicted in SEQ ID NO: 123, and CDR-H3 as depicted in SEQ ID NO: 124, and/or a VL region comprising CDR-L1 as depicted in SEQ ID NO: 125, CDR-L2 as depicted in SEQ ID NO: 126 and CDR-L3 as depicted in SEQ ID NO: 127;
   b) a VH region comprising CDR-H1 as depicted in SEQ ID NO: 128, CDR-H2 as depicted in SEQ ID NO: 129, and CDR-H3 as depicted in SEQ ID NO: 130, and/or a VL region comprising CDR-L1 as depicted in SEQ ID NO: 131, CDR-L2 as depicted in SEQ ID NO: 132 and CDR-L3 as depicted in SEQ ID NO: 133;
   c) a VH region comprising CDR-H1 as depicted in SEQ ID NO: 134, CDR-H2 as depicted in SEQ ID NO: 135, and CDR-H3 as depicted in SEQ ID NO: 136, and/or a VL region comprising CDR-L1 as depicted in SEQ ID NO: 137, CDR-L2 as depicted in SEQ ID NO: 138 and CDR-L3 as depicted in SEQ ID NO: 139;
   d) a VH region comprising CDR-H1 as depicted in SEQ ID NO: 140, CDR-H2 as depicted in SEQ ID NO: 141, and CDR-H3 as depicted in SEQ ID NO: 142, and/or a VL region comprising CDR-L1 as depicted in SEQ ID NO: 143, CDR-L2 as depicted in SEQ ID NO: 144 and CDR-L3 as depicted in SEQ ID NO: 145;
   e) a VH region comprising CDR-H1 as depicted in SEQ ID NO: 146, CDR-H2 as depicted in SEQ ID NO: 147, and CDR-H3 as depicted in SEQ ID NO: 148, and/or a VL region comprising CDR-L1 as depicted in SEQ ID NO: 149, CDR-L2 as depicted in SEQ ID NO: 150 and CDR-L3 as depicted in SEQ ID NO: 151;
   f) a VH region comprising CDR-H1 as depicted in SEQ ID NO: 152, CDR-H2 as depicted in SEQ ID NO: 153, and CDR-H3 as depicted in SEQ ID NO: 154, and/or a VL region comprising CDR-L1 as depicted in SEQ ID NO: 155, CDR-L2 as depicted in SEQ ID NO: 156 and CDR-L3 as depicted in SEQ ID NO: 157;
   g) a VH region comprising CDR-H1 as depicted in SEQ ID NO: 158, CDR-H2 as depicted in SEQ ID NO: 159, and CDR-H3 as depicted in SEQ ID NO: 160, and/or a VL region comprising CDR-L1 as depicted in SEQ ID NO: 161, CDR-L2 as depicted in SEQ ID NO: 162 and CDR-L3 as depicted in SEQ ID NO: 163; or
   h) a VH region comprising CDR-H1 as depicted in SEQ ID NO: 164, CDR-H2 as depicted in SEQ ID NO: 165, and CDR-H3 as depicted in SEQ ID NO: 166, and/or a VL region comprising CDR-L1 as depicted in SEQ ID NO: 167, CDR-L2 as depicted in SEQ ID NO: 168 and CDR-L3 as depicted in SEQ ID NO: 169.

In one embodiment, the above described antibody or antibody construct which binds to PD-1 comprises
a) a VH region as depicted in SEQ ID NO: 170, and a VL region as depicted in SEQ ID NO: 171;
b) a VH region as depicted in SEQ ID NO: 172, and a VL region as depicted in SEQ ID NO: 173;
c) a VH region as depicted in SEQ ID NO: 174, and a VL region as depicted in SEQ ID NO: 175;
d) a VH region as depicted in SEQ ID NO: 176, and a VL region as depicted in SEQ ID NO: 177;
e) a VH region as depicted in SEQ ID NO: 178, and a VL region as depicted in SEQ ID NO: 179;
f) a VH region as depicted in SEQ ID NO: 180, and a VL region as depicted in SEQ ID NO: 181;
g) a VH region as depicted in SEQ ID NO: 182, and a VL region as depicted in SEQ ID NO: 183; or
h) a VH region as depicted in SEQ ID NO: 184, and a VL region as depicted in SEQ ID NO: 185.

In one embodiment, the above antibody or antibody construct which binds to PD-1 comprises:
a) a heavy chain as depicted in SEQ ID NO: 186, and a light chain as depicted in SEQ ID NO: 187;
b) a heavy chain as depicted in SEQ ID NO: 188, and a light chain as depicted in SEQ ID NO: 189;
c) a heavy chain as depicted in SEQ ID NO: 190, and a light chain as depicted in SEQ ID NO: 191;
d) a heavy chain as depicted in SEQ ID NO: 192, and a light chain as depicted in SEQ ID NO: 193;
e) a heavy chain as depicted in SEQ ID NO: 194, and a light chain as depicted in SEQ ID NO: 195;
f) a heavy chain as depicted in SEQ ID NO: 196, and a light chain as depicted in SEQ ID NO: 197;
g) a heavy chain as depicted in SEQ ID NO: 198, and a light chain as depicted in SEQ ID NO: 199; or
h) a heavy chain as depicted in SEQ ID NO: 200, and a light chain as depicted in SEQ ID NO: 201.

In certain embodiments, the optimal pharmaceutical composition is determined depending upon, for example, the intended route of administration, delivery format and desired dosage. See, for example, Remington's Pharmaceutical Sciences, supra. In certain embodiments, such compositions may influence the physical state, stability, rate of in vivo release and rate of in vivo clearance of the antibody construct used in accordance with the invention. In certain embodiments, the primary vehicle or carrier in a pharmaceutical composition may be either aqueous or non-aqueous in nature. For example, a suitable vehicle or carrier may be water for injection or physiological saline solution, possibly supplemented with other materials common in compositions for parenteral administration. In certain embodiments, the compositions comprising the antibody construct used in accordance with the invention may be prepared for storage by mixing the selected composition having the desired degree of purity with optional formulation agents (Remington's Pharmaceutical Sciences, supra) in the form of a lyophilized cake or an aqueous solution. Further, in certain embodiments, the antibody construct used in accordance with the invention may be formulated as a lyophilizate using appropriate excipients.

When parenteral administration is contemplated, the therapeutic compositions used in this invention may be provided in the form of a pyrogen-free, parenterally acceptable aqueous solution comprising the desired antibody construct used in accordance with the invention in a pharmaceutically acceptable vehicle. A particularly suitable vehicle for parenteral injection is sterile distilled water in which the antibody construct used in accordance with the invention is formulated as a sterile, isotonic solution, properly preserved. In certain embodiments, the preparation can involve the formulation of the desired molecule with an agent that may provide controlled or sustained release of the product which can be delivered via depot injection, or that may promote sustained duration in the circulation. In certain embodiments, implantable drug delivery devices may be used to introduce the desired antibody construct.

Additional pharmaceutical compositions will be evident to those skilled in the art, including formulations including the antibody construct used in accordance with the invention in sustained or controlled delivery formulations. Techniques for formulating a variety of sustained- or controlled-delivery means are known to those skilled in the art. The antibody construct may also be entrapped in microcapsules prepared, for example, by coacervation techniques or by interfacial polymerization, in colloidal drug delivery systems, or in macroemulsions. Such techniques are disclosed in Remington's Pharmaceutical Sciences, supra.

Pharmaceutical compositions used for in vivo administration are typically provided as sterile preparations. Sterilization can be accomplished by filtration through sterile filtration membranes. When the composition is lyophilized, sterilization using this method may be conducted either prior to or following lyophilization and reconstitution. Compositions for parenteral administration can be stored in lyophilized form or in a solution. Parenteral compositions are generally placed into a container having a sterile access port, for example, an intravenous solution bag or vial having a stopper pierceable by a hypodermic injection needle.

Another aspect of the invention includes self-buffering formulations comprising the antibody construct used in accordance with the invention, which can be used as pharmaceutical compositions, as described in international patent application WO 2006/138181. A variety of publications are available on protein stabilization and formulation materials and methods useful in this regard, such as Arawaka T. et al., Pharm Res. 1991 March; 8(3):285-91; Kendrick et al., “Physical stabilization of proteins in aqueous solution” in: Rational Design of Stable Protein Formulations: Theory and Practice, Carpenter and Manning, eds. Pharmaceutical Biotechnology. 13: 61-84 (2002), and Randolph and Jones, Pharm Biotechnol. 2002; 13:159-75, see particularly the parts pertinent to excipients and processes for self-buffering protein formulations, especially as to protein pharmaceutical products and processes for veterinary and/or human medical uses.

Salts may be used in accordance with certain embodiments of the invention, e.g. to adjust the ionic strength and/or the isotonicity of a composition or formulation and/or to improve the solubility and/or physical stability of an antibody construct or other ingredient of a composition in accordance with the invention. Ions can stabilize the native state of proteins by binding to charged residues on the protein's surface and by shielding charged and polar groups in the protein and reducing the strength of their electrostatic interactions, attractive, and repulsive interactions. Ions also can stabilize the denatured state of a protein by binding to, particularly the denatured peptide linkages (—CONH) of the protein. Furthermore, ionic interaction with charged and polar groups in a protein also can reduce intermolecular electrostatic interactions and, thereby, prevent or reduce protein aggregation and insolubility.

Ionic species differ significantly in their effects on proteins. Several categorical rankings of ions and their effects on proteins have been developed that can be used in formulating pharmaceutical compositions in accordance with the invention. One example is the Hofmeister series, which ranks ionic and polar non-ionic solutes by their effect on the conformational stability of proteins in solution. Stabilizing solutes are referred to as “kosmotropic”. Destabilizing solutes are referred to as “chaotropic”. Kosmotropes are commonly used at high concentrations to precipitate proteins from solution (“salting-out”). Chaotropes are commonly used to denature and/or to solubilize proteins (“salting-in”). The relative effectiveness of ions to “salt-in” and “salt-out” defines their position in the Hofmeister series.

Free amino acids can be used in formulations or compositions comprising the antibody construct used in accordance with the invention in accordance with various embodiments of the invention as bulking agents, stabilizers, and antioxidants, as well as for other standard uses. Certain amino acids can be used for stabilizing proteins in a formulation, others are useful during lyophilization to ensure correct cake structure and properties of the active ingredient. Some amino acids may be useful to inhibit protein aggregation in both liquid and lyophilized formulations, and others are useful as antioxidants.

Polyols are kosmotropic and are useful as stabilizing agents in both liquid and lyophilized formulations to protect proteins from physical and chemical degradation processes. Polyols are also useful for adjusting the tonicity of formulations and for protecting against freeze-thaw stresses during transport or the preparation of bulks during the manufacturing process. Polyols can also serve as cryoprotectants in the context of the present invention.

Certain embodiments of the formulation or composition comprising the antibody construct used in accordance with the invention can comprise surfactants. Proteins may be susceptible to adsorption on surfaces and to denaturation and resulting aggregation at air-liquid, solid-liquid, and liquid-liquid interfaces. These deleterious interactions generally scale inversely with protein concentration and are typically exacerbated by physical agitation, such as that generated during the shipping and handling of a product. Surfactants are routinely used to prevent, minimize, or reduce surface adsorption. Surfactants also are commonly used to control protein conformational stability. The use of surfactants in this regard is protein specific, since one specific surfactant will typically stabilize some proteins and destabilize others.

Certain embodiments of the formulation or composition comprising the antibody construct used in accordance with the invention can comprise one or more antioxidants. To some extent deleterious oxidation of proteins can be prevented in pharmaceutical formulations by maintaining proper levels of ambient oxygen and temperature and by avoiding exposure to light. Antioxidant excipients can also be used to prevent oxidative degradation of proteins. It is envisaged that antioxidants used in therapeutic protein formulations in accordance with the present invention can be water-soluble and maintain their activity throughout the shelf life of the product (the composition comprising the antibody construct). Antioxidants can also damage proteins and should hence—among other things—be selected in a way to eliminate or sufficiently reduce the possibility of antioxidants damaging the antibody construct or other proteins in the formulation.

Certain embodiments of the formulation or composition comprising the antibody construct used in accordance with the invention can comprise one or more preservatives. Preservatives are necessary for example when developing multi-dose parenteral formulations that involve more than one extraction from the same container. Their primary function is to inhibit microbial growth and ensure product sterility throughout the shelf-life or term of use of the drug product. Although preservatives have a long history of use with small-molecule parenterals, the development of protein formulations that include preservatives can be challenging. Preservatives very often have a destabilizing effect (aggregation) on proteins, and this has become a major factor in limiting their use in multi-dose protein formulations. To date, most protein drugs have been formulated for single-use only. However, when multi-dose formulations are possible, they have the added advantage of enabling patient convenience, and increased marketability. A good example is that of human growth hormone (hGH) where the development of preserved formulations has led to commercialization of more convenient, multi-use injection pen presentations. Several aspects need to be considered during the formulation and development of preserved dosage forms. The effective preservative concentration in the drug product must be optimized. This requires testing a given preservative in the dosage form with concentration ranges that confer anti-microbial effectiveness without compromising protein stability.

As might be expected, development of liquid formulations containing preservatives are more challenging than lyophilized formulations. Freeze-dried products can be lyophilized without the preservative and reconstituted with a preservative containing diluent at the time of use. This shortens the time during which a preservative is in contact with the antibody construct, significantly minimizing the associated stability risks. With liquid formulations, preservative effectiveness and stability should be maintained over the entire product shelf-life. An important point to note is that preservative effectiveness should be demonstrated in the final formulation containing the active drug and all excipient components. Once the pharmaceutical composition has been formulated, it may be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration.

The biological activity of the pharmaceutical composition defined herein can be determined for instance by cytotoxicity assays, as described in the following examples, in WO 99/54440 or by Schlereth et al. (Cancer Immunol. Immunother. 20 (2005), 1-12). “Efficacy” or “in vivo efficacy” as used herein refers to the response to therapy by the pharmaceutical composition of formulation of the invention, using e.g. standardized NCI response criteria. The success or in vivo efficacy of the therapy using a pharmaceutical composition of the invention refers to the effectiveness of the composition for its intended purpose, i.e. the ability of the composition to cause its desired effect, i.e. depletion of pathologic cells, e.g. tumor cells. The in vivo efficacy may be monitored by established standard methods for the respective disease entities including, but not limited to, white blood cell counts, differentials, fluorescence activated cell sorting, bone marrow aspiration. In addition, various disease specific clinical chemistry parameters and other established standard methods may be used. Furthermore, computer-aided tomography, X-ray, nuclear magnetic resonance tomography, positron-emission tomography scanning, lymph node biopsies/histologies and other established standard methods may be used.

Another major challenge in the development of drugs such as the pharmaceutical composition of the invention is the predictable modulation of pharmacokinetic properties. To this end, a pharmacokinetic profile of the drug candidate, i.e. a profile of the pharmacokinetic parameters that affect the ability of a specific drug to treat a given condition, can be established. Pharmacokinetic parameters of the drug influencing the ability of a drug for treating a certain disease entity include, but are not limited to: half-life, volume of distribution, hepatic first-pass metabolism and the degree of blood serum binding. The efficacy of a given drug agent can be influenced by each of the parameters mentioned above.

“Half-life” is the time required for a quantity to reduce to half its initial value. The medical sciences refer to the half-life of substances or drugs in the human body. In a medical context, half-life may refer to the time it takes for a substance/drug to lose one-half of its activity, e.g. pharmacologic, physiologic, or radiological activity. The half-life may also describe the time that it takes for the concentration of a drug or substance (e.g., an antibody construct used in accordance with the invention) in blood plasma/serum to reach one-half of its steady-state value (“serum half-life”). Typically, the elimination or removal of an administered substance/drug refers to the body's cleansing through biological processes such as metabolism, excretion, also involving the function of kidneys and liver. The “first-pass metabolism” is a phenomenon of drug metabolism whereby the concentration of a drug is reduced before it reaches the circulation. It is the fraction of drug lost during the process of absorption. Accordingly, by “hepatic first-pass metabolism” is meant the propensity of a drug to be metabolized upon first contact with the liver, i.e. during its first pass through the liver. “Volume of distribution” (V_{D}) means the degree to which a drug is distributed in body tissue rather than the blood plasma, a higher V_{D} indicating a greater amount of tissue distribution. The retention of a drug can occur throughout the various compartments of the body, such as intracellular and extracellular spaces, tissues and organs, etc. “Degree of blood serum binding” means the propensity of a drug to interact with and bind to blood serum proteins, such as albumin, leading to a reduction or loss of biological activity of the drug.

Pharmacokinetic parameters also include bioavailability, lag time (T lag), Tmax, absorption rates, and/or Cmax for a given amount of drug administered. “Bioavailability” refers to the fraction of an administered dose of a drug/substance that reaches the systemic circulation (the blood compartment). When a medication is administered intravenously, its bioavailability is considered to be 100%. However, when a medication is administered via other routes (such as orally), its bioavailability generally decreases. “Lag time” means the time delay between the administration of the drug and its detection and measurability in blood or plasma. Cmax is the maximum plasma concentration that a drug achieves after its administration (and before the administration of a second dose). Tmax is the time at which Cmax is reached. The time to reach a blood or tissue concentration of the drug which is required for its biological effect is influenced by all parameters. Pharmacokinetic parameters of antibody constructs exhibiting cross-species specificity may be determined in preclinical animal testing in non-chimpanzee primates as outlined above and set forth e.g. in Schlereth et al. (supra).

One embodiment provides the antibody construct used in accordance with the invention (or the antibody construct produced according to the process disclosed herein) used in the prevention, treatment or amelioration of a disease, preferably a neoplasm. Another embodiment provides the use of the antibody construct used in accordance with the invention in the manufacture of a medicament for the prevention, treatment or amelioration of a disease, preferably a neoplasm. It is also envisaged to provide a method for the prevention, treatment or amelioration of a disease, preferably a neoplasm, comprising the step of administering to a subject in need thereof the antibody construct. The terms “subject in need”, “patient” or those “in need of treatment” include those already with the disease, as well as those in which the disease is to be prevented. The terms also include human and other mammalian subjects that receive either prophylactic or therapeutic treatment unless the species or genus is specifically indicated. In particular embodiments of all other embodiments or claims herein, the term “patient” relates to human patients and/or non-human primates.

The antibody constructs used in accordance with the invention and the formulations/pharmaceutical compositions described herein are useful in the treatment, amelioration and/or prevention of the medical condition as described herein in a patient in need thereof. The term “treatment” refers to both therapeutic treatment and prophylactic or preventative measures. Treatment includes the application or administration of the antibody constructs/pharmaceutical composition to the body, to an isolated tissue, or to a cell from a patient or a subject in need who has a disease/disorder as described herein, a symptom of such disease/disorder, or a predisposition toward such disease/disorder, with the purpose to cure, heal, alleviate, relieve, alter, remedy, ameliorate, improve, or affect the disease, the symptom of the disease, or the predisposition toward the disease. The term “amelioration” as used herein refers to any improvement of the disease state of a patient, by the administration of an antibody construct according to the invention to such patient or subject in need thereof. Such an improvement may be a slowing down or stopping of the progression of the disease of the patient, and/or as a decrease in severity of disease symptoms, an increase in frequency or duration of disease symptom-free periods or a prevention of impairment or disability due to the disease. The term “prevention” as used herein means the avoidance of the occurrence or of the re-occurrence of a disease as specified herein, by the administration of an antibody construct used in accordance with the invention to a subject in need thereof. Further, a “patient at risk of developing an adverse event” or “patient with an intolerance to” a given substance/drug includes patients that are known to have previously reacted adversely to such given substance/drug. Also encompassed can be patients having a total B cell count of less than about 50 B cells/microliter of peripheral blood, which are known as susceptible to potential adverse effects also including the onset of an adverse effect to immunotherapy with CD3-binding antibody-based molecules, particularly with blinatumomab, while a patient who has a total B cell count of greater than about 50 B cells/microliter of peripheral blood does not have (is not at) or at least has (is at) a decreased risk of potential adverse effects. Also patients with a B:T cell ratio that is lower than 1:5 to 1:10, particularly lower than 1:5 are often at higher risk of developing adverse events to immunotherapy with CD3-binding antibody-based molecules. Such patients form are particular group that profits from the herein described methods of preventing adverse effects and is a particular group that is contemplated for a preventive method using the herein described TNF/TNFR signaling antagonists or inhibitors. To determine the B cell numbers or the B:T ratio, A sample which includes peripheral blood mononuclear cells (PBMCs), in particular B cells and T cells is preferably taken from peripheral blood of a patient. The B:T cell ratio of the patient population treated according to the present disclosure may be about 1:5 or lower including a B:T cell ratio of about 1:6, 1:7, 1:8, 1:9, 1:10, 1:11, 1:12, 1:13, 1:14, 1:15, 1:20, 1:100, 1:200, 1:400, 1:500, 1:1000, 1:2000, 1:3000, 1:4000, 1:5000 or even lower, with less than about 1:8, 1:9, 1:10, 1:50, 1:100, 1:500, 1:1000 being indicative for a risk of potential adverse effects for said patient.

“Determining the B:T cell ratio” includes

(a) determining the total B cell number in a sample from a patient, preferably in a peripheral blood sample of the patient;
(b) determining the total T cell number in sample from a patient, preferably in a peripheral blood sample of the patient; and
(c) calculating the ratio of the B cell number of step (a) and the T cell number of step (b) in order to obtain a B:T cell ratio.

Of note, a low B:T cell ratio can also be seen as high T:B ratio; and vice versa. Accordingly, the ratios provided herein for a low B:T cell ratio would then have to be reversed.

The term “disease” refers to any condition that would benefit from treatment with the antibody construct or the pharmaceutical composition described herein. This includes chronic and acute disorders or diseases including those pathological conditions that predispose the mammal to the disease in question. The disease is preferably a neoplasm, cancer or tumor. The disease, neoplasm, cancer or tumor is preferably the target antigen(s) positive, i.e. it is characterized by expression or overexpression of the target antigen(s).

A “neoplasm” is an abnormal growth of tissue, usually but not always forming a mass. When also forming a mass, it is commonly referred to as a “tumor”. Neoplasms or tumors can be benign, potentially malignant (pre-cancerous), or malignant (cancerous). Malignant neoplasms/tumors are commonly called cancer. They usually invade and destroy the surrounding tissue and may form metastases, i.e., they spread to other parts, tissues or organs of the body. A “primary tumor” is a tumor growing at the anatomical site where tumor progression began and proceeded to yield a cancerous mass. Most cancers develop at their primary site but then go on to metastasize or spread to other parts (e.g. tissues and organs) of the body. These further tumors are “secondary tumors”. Most cancers continue to be called after their primary site, even after they have spread to other parts of the body.

Lymphomas and leukemias are lymphoid neoplasms. For the purposes of the present invention, they are also encompassed by the terms “tumor” or “cancer”. For the purposes of the present invention, the terms “neoplasm”, “tumor” and “cancer” may be used interchangeably, and they comprise both primary tumors/cancers and secondary tumors/cancers (or “metastases”) as well as mass-forming neoplasms (tumors) and lymphoid neoplasms (such as lymphomas and leukemias), and minimal residual disease (MRD).

The term “minimal residual disease” (MRD) refers to the evidence for the presence of small numbers of residual cancer cells that remain in the patient after cancer treatment, e.g. when the patient is in remission (no symptoms or signs of disease). A very small number of remaining cancer cells usually cannot be detected by routine means because the standard tests used to assess or detect cancer are not sensitive enough to detect MRD. Nowadays, very sensitive molecular biology tests for MRD are available, such as flow cytometry, PCR and next-generation sequencing. These tests can measure minimal levels of cancer cells in tissue samples, sometimes as low as one cancer cell in a million normal cells. In the context of the present invention, the terms “prevention”, “treatment” or “amelioration” of a cancer are envisaged to also encompass “prevention, treatment or amelioration of MRD”, whether the MRD was detected or not.

In one embodiment of the invention, the neoplasm, cancer or tumor is selected from the group comprising: carcinoma, sarcoma, myeloma, leukemia, or lymphoma, including, but not limited to, (or consisting of) ovarian cancer, uterine cancer, germinal cancer, breast cancer, brain cancer, prostate cancer, pancreas cancer, liver cancer, colon cancer, intestinal cancer, bone cancer, mouth, gastric cancer, bone cancer, mouth cancer, esophagus cancer, leukemia, melanoma, renal carcinoma, bladder carcinoma, small cell carcinoma, head and neck cancer, and lung cancer.

The antibody construct used in accordance with the invention will generally be designed for specific routes and methods of administration, for specific dosages and frequencies of administration, for specific treatments of specific diseases, with ranges of bio-availability and persistence, among other things. The materials of the composition are preferably formulated in concentrations that are acceptable for the site of administration. Formulations and compositions thus may be designed in accordance with the invention for delivery by any suitable route of administration. In the context of the present invention, the routes of administration include, but are not limited to topical routes, enteral routes and parenteral routes.

If the pharmaceutical composition has been lyophilized, the lyophilized material is first reconstituted in an appropriate liquid prior to administration. The lyophilized material may be reconstituted in, e.g., bacteriostatic water for injection (BWFI), physiological saline, phosphate buffered saline (PBS), or the same formulation the protein had been in prior to lyophilization. The pharmaceutical compositions and the antibody construct used in accordance with this invention are particularly useful for parenteral administration, e.g., intravenous delivery, for example by injection or infusion. Pharmaceutical compositions may be administered using a medical device. Examples of medical devices for administering pharmaceutical compositions are described in U.S. Pat. Nos. 4,475,196; 4,439,196; 4,447,224; 4,447, 233; 4,486,194; 4,487,603; 4,596,556; 4,790,824; 4,941,880; 5,064,413; 5,312,335; 5,312,335; 5,383,851; and 5,399,163.

The compositions of the present invention can be administered to the subject at a suitable dose which can be determined e.g. in dose escalating studies. As set forth above, the antibody construct used in accordance with the invention exhibiting cross-species specificity as described herein can also be advantageously used in in preclinical testing in non-chimpanzee primates. The dosage regimen will be determined by the attending physician and clinical factors. As is well known in the medical art, dosages for any one patient depend upon many factors, including the patient's size, body surface area, age, the specific compound to be administered, sex, time and route of administration, general health, and other drugs being administered concurrently.

An “effective dose” is an amount of a therapeutic agent that is sufficient to achieve or at least partially achieve a desired effect. A “therapeutically effective dose” is an amount that is sufficient to cure or at least partially arrest the disease and its complications, signs and symptoms in a patient suffering from the disease. Amounts or doses effective for this use will depend on the disease to be treated (the indication), the delivered antibody construct, the therapeutic context and objectives, the severity of the disease, prior therapy, the patient's clinical history and response to the therapeutic agent, the route of administration, the size (body weight, body surface) and/or condition (the age and general health) of the patient, and the general state of the patient's own immune system. The proper dose can be adjusted according to the judgment of the attending physician, to obtain the optimal therapeutic effect.

A therapeutically effective amount of an antibody construct used in accordance with the invention preferably results in a decrease in severity of disease symptoms, an increase in frequency or duration of disease symptom-free periods or a prevention of impairment or disability due to the disease. In the treatment of the target antigen(s)-expressing tumors, a therapeutically effective amount of the antibody construct of the invention preferably inhibits tumor cell growth by at least about 20%, at least about 40%, at least about 50%, at least about 60%, at least about 70%, at least about 80%, or at least about 90% relative to untreated patients. The ability of a compound to inhibit tumor growth may also be evaluated in an animal model predictive of efficacy in human tumors.

In a further embodiment, the invention provides a kit comprising an antibody construct used in accordance with the invention, an antibody construct produced according to the process disclosed herein, a polynucleotide disclosed herein, a vector disclosed herein, and/or a host cell disclosed herein. In the context of the present invention, the term “kit” means two or more components—one of which corresponding to the antibody construct, the pharmaceutical composition, the polynucleotide, the vector or the host cell of the invention—packaged together in a container, recipient or otherwise. A kit can hence be described as a set of products and/or utensils that are sufficient to achieve a certain goal, which can be marketed as a single unit. In a preferred embodiment of the present invention, the kit comprises an antibody construct as defined above as well as an inhibitor/antagonist of TNFa/TNFaR-signalling (for example, etanercept).

It is envisaged that a further component of the kit of the invention is an agent, preferably an antibody or antibody construct, which binds to a protein of the immune checkpoint pathway (such as PD-1 or CTLA-4) or to a co-stimulatory immune checkpoint receptor (such as 4-1BB). These agents are described in more detail herein above. According to one embodiment, the kit comprises an antibody construct as defined herein, an inhibitor/antagonist of TNFa/TNFaR-signalling (for example, etanercept), and an antibody or antibody construct which binds to PD-1. Anti-PD-1 binding proteins useful for this purpose are e.g. described in detail in PCT/US2019/013205. In certain embodiment, the kit allows for the simultaneous and/or sequential administration of the components.

The kit may comprise one or more recipients (such as vials, ampoules, containers, syringes, bottles, bags) of any appropriate shape, size and material (preferably waterproof, e.g. plastic or glass) containing the antibody construct or the pharmaceutical composition of the present invention in an appropriate dosage for administration (see above). The kit may additionally contain directions used (e.g. in the form of a leaflet or instruction manual), means for administering the antibody construct or the pharmaceutical composition of the present invention such as a syringe, pump, infuser or the like, means for reconstituting the antibody construct as defined above, the inhibitor/antagonist of TNFa/TNFaR-signalling (for example, etanercept) and/or means for diluting the antibody construct used in accordance with the invention.

The invention also provides kits for a single-dose administration unit. The kit of the invention may also contain a first recipient comprising a dried/lyophilized antibody construct or pharmaceutical composition, a second recipient comprising an inhibitor/antagonist of TNFa/TNFaR-signalling (for example, etanercept) and a third recipient comprising an aqueous formulation. In certain embodiments of this invention, kits containing single-chambered and multi-chambered pre-filled syringes are provided.

### Further General Definitions

As used herein, the singular forms “a”, “an”, and “the” include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within ±20%, preferably within ±15%, more preferably within ±10%, and most preferably within ±5% of a given value or range. It also includes the concrete value, e.g., “about 50” includes the value “50”.

Throughout this specification and the claims, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein, any of the terms “comprising”, “consisting essentially of” and “consisting of” may be replaced with either of the other two terms.

The above description and the below examples provide exemplary arrangements, but the present invention is not limited to the specific methodologies, techniques, protocols, material, reagents, substances, etc., described herein and as such can vary. The terminology used herein serves to describe specific embodiments only. The terminology used herein does not intend to limit the scope of the present invention, which is defined solely by the claims. Aspects of the invention are provided in the independent claims. Some optional features of the invention are provided in the dependent claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

A better understanding of the present invention and of its advantages will be obtained from the following examples, offered for illustrative purposes only. The examples are not intended and should not be construed as to limit the scope of the present invention in any way.

### DESCRIPTION OF THE FIGURES

FIG. 1—Pretreatment with TNFα blockade did not affect BiTE® molecule efficacy: Human CD3c knock-in mice were pretreated with either a control antibody, TNFα blockade agents (anti-TNFα antibody or a TNFRII-Fc) administered once per day for two days prior to dosing either a negative control BiTE® molecule (1000 μg/kg) or a mouse CD19 BiTE® molecule (100 μg/kg or 1000 μg/kg) that recognizes CD19 on B cells (n=5 animals per group). At 72 hours post-BiTE® dose, animals were euthanized, and spleens were harvested for analysis. B cells were enumerated by flow cytometry as described in Methods. The engagement of T cells in vivo using a murine CD19 targeted BiTE® molecule resulted in depletion of B cells in the spleen, and administration of TNFα blocking reagents prior to BiTE® administration did not affect the ability of the BiTE® molecule to mediate depletion of splenic B cells. Significance values: ns, p>0.05; * p≤0.05, ** p≤0.01, *** p≤0.001, **** p≤0.0001 (one-way ANOVA with Tukey's test); TNFα=tumor necrosis factor alpha, BiTE®=bispecific T cell engager, anti-TNFα Ab=TNFα blocking antibody, TNFRII-Fc=tumor necrosis factor receptor II-Fc fusion protein.

FIG. 1. BiTE® molecule-induced serum IFNγ and IL-2 were not significantly reduced by TNFα blockade prior to BiTE® molecule administration: Human CD3c knock-in mice were pretreated with either a control antibody, TNFα blockade agents (anti-TNFα antibody or a TNFRII-Fc) for two days prior to dosing with either a negative control BiTE® molecule (1000 μg/kg) or a mouse CD19 BiTE® molecule (100 μg/kg and 1000 μg/kg) that recognizes CD19 on B cells (n=5 animals per group). Serum IFNγ and IL-2 were measured four hours after BiTE® treatment. Mean (A) IFNγ, (B) IL-2 levels in groups pretreated with both TNFα blocking agents were not significantly lower after administration of CD19 BiTE® at both doses compared to control antibody pretreatment groups (one-way ANOVA with Tukey's test). Significance values: ns, p>0.05; * p≤0.05, ** p≤0.01, *** p≤0.001, **** p≤0.0001; IFNγ=interferon gamma, IL-2=interleukin 2, BiTE®=bispecific T cell engager, TNFα=tumor necrosis factor alpha, anti-TNFα Ab=TNFα blocking antibody, TNFRII-Fc=TNF receptor II Fc-fusion protein, ULOD=upper limit of detection.

FIG. 3. BiTE® molecule-induced Serum IL-6 was significantly reduced by TNFα blockade prior to BiTE® administration: Human CD3c knock-in mice were pretreated with either a control antibody, TNFα blockade agents (anti-TNFα antibody or a TNFRII-Fc) for two days prior to dosing with either a negative control BiTE® molecule (1000 μg/kg) or a mouse CD19 BiTE® molecule (100 μg/kg and 1000 μg/kg) that recognizes CD19 on B cells (n=5 animals per group). Serum IL-6 was measured four hours after BiTE® treatment. Mean IL-6 levels in groups pretreated with both TNFα blocking agents were significantly lower after administration of CD19 BiTE® at both doses compared to control antibody pretreatment groups (one-way ANOVA with Tukey's test). Significance values: ns, p>0.05; * p≤0.05, ** p≤0.01, *** p≤0.001, **** p≤0.0001; IL-6=interleukin 6, BiTE®=bispecific T cell engager, TNFα=tumor necrosis factor alpha, anti-TNFα Ab=TNFα blocking antibody, TNFRII-Fc=TNF receptor II Fc-fusion protein, ULOD=upper limit of detection.

FIG. 4. Cytokine measurement in Example 2: Blood samples of 5 animals were collected 4 and 24 hours after each vehicle and muS110 administration, respectively and TNF, IL-6 and MCP1 serum concentrations were determined using the BD™ CBA Mouse Flex Set System (BD) in accordance to the manufactures instructions.

FIG. 5. Impact of TNF Blockage on AMG 110 Bioactivity

FIG. 6. AMG 103-mediated MCP-1 Secretion: MCP-1 concentrations in cell culture supernatants were quantified by the BD Cytometric CBA human MCP-1 Flex Set. Error bars in both graphs indicate the standard error of the mean of duplicate measurements.

FIG. 7. VCAM-1 Expression on HUVEC in AMG 103 Co-culture Assays: VCAM-1 expression by flow cytometry. ICAM-1 expression levels are expressed as Median Fluorescence Intensity (MFI). VCAM-1 expressing HUVEC are expressed as percentage of CD31+HUVEC. Error bars indicate the standard error of the mean of duplicate measurements. Two PBMC donors were used in this experiment.

### EXAMPLE 1

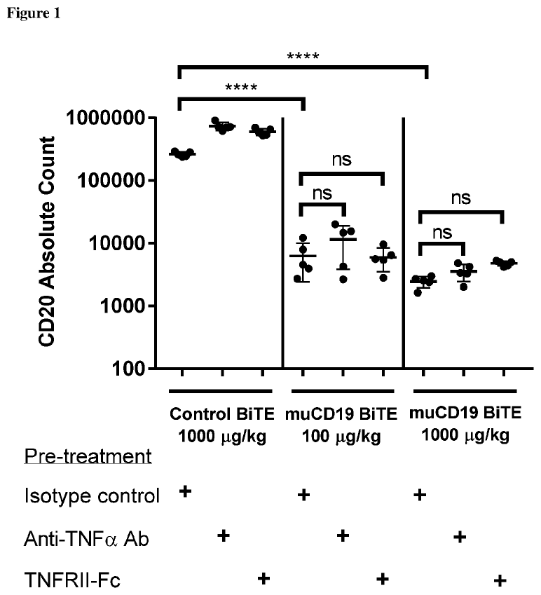

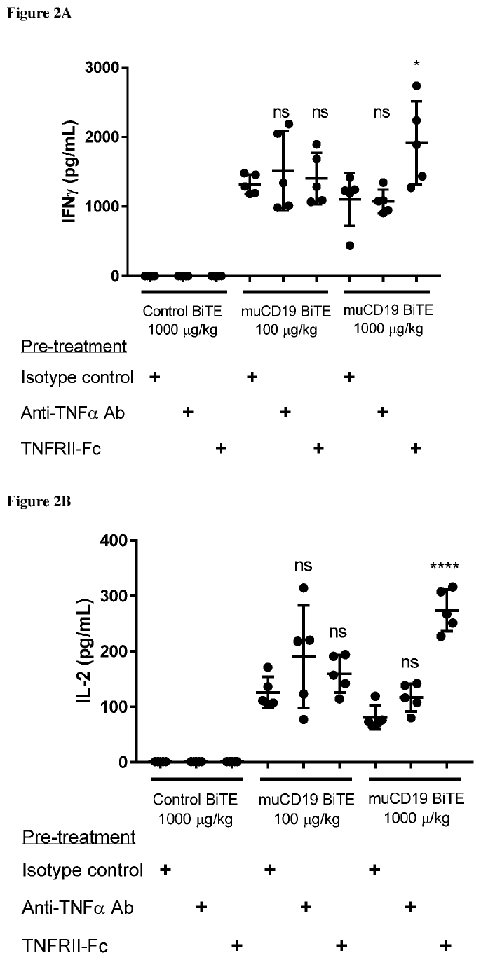

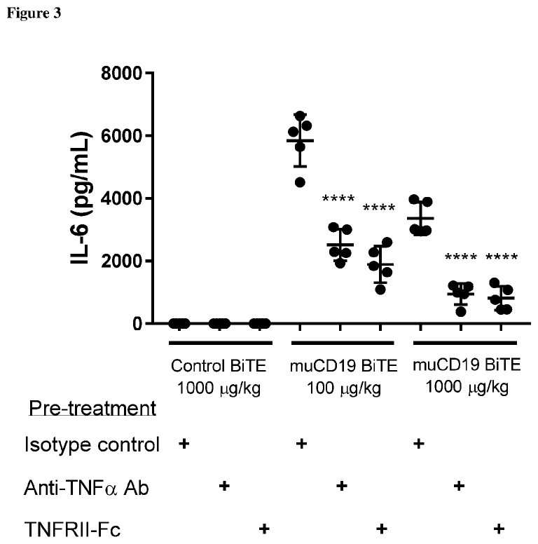

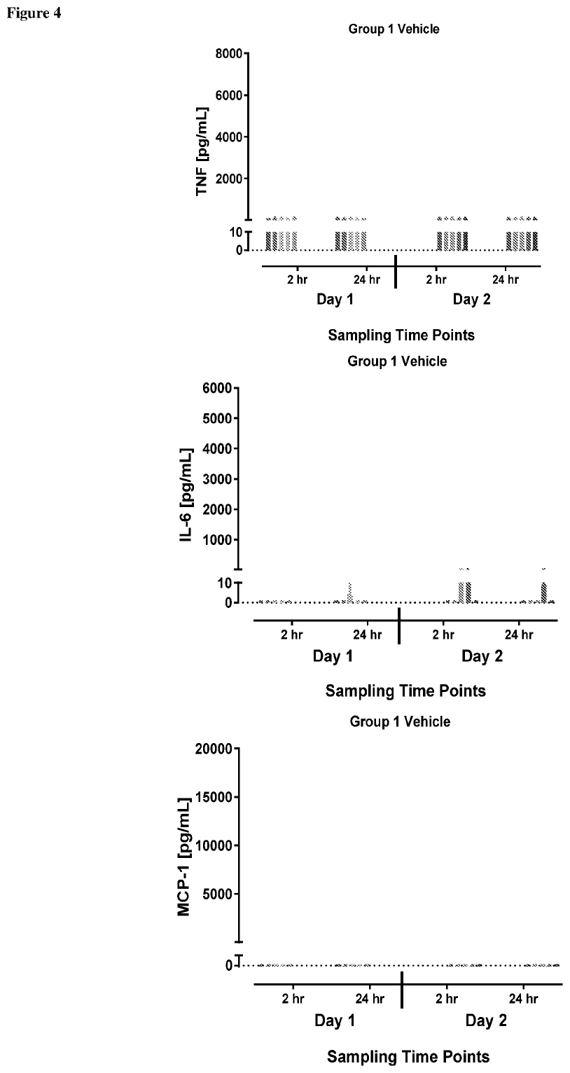

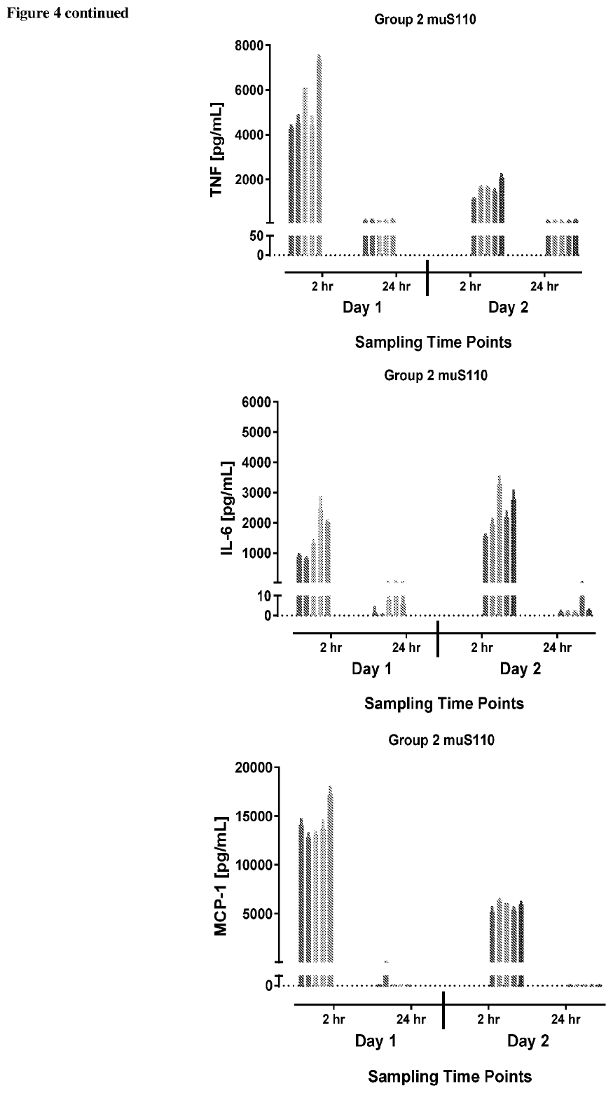

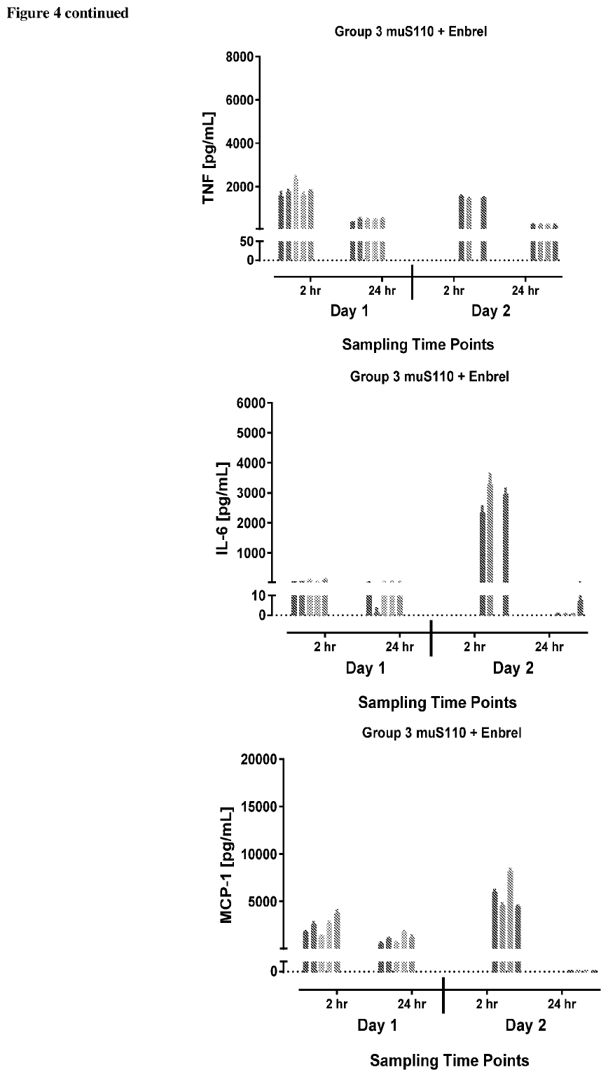

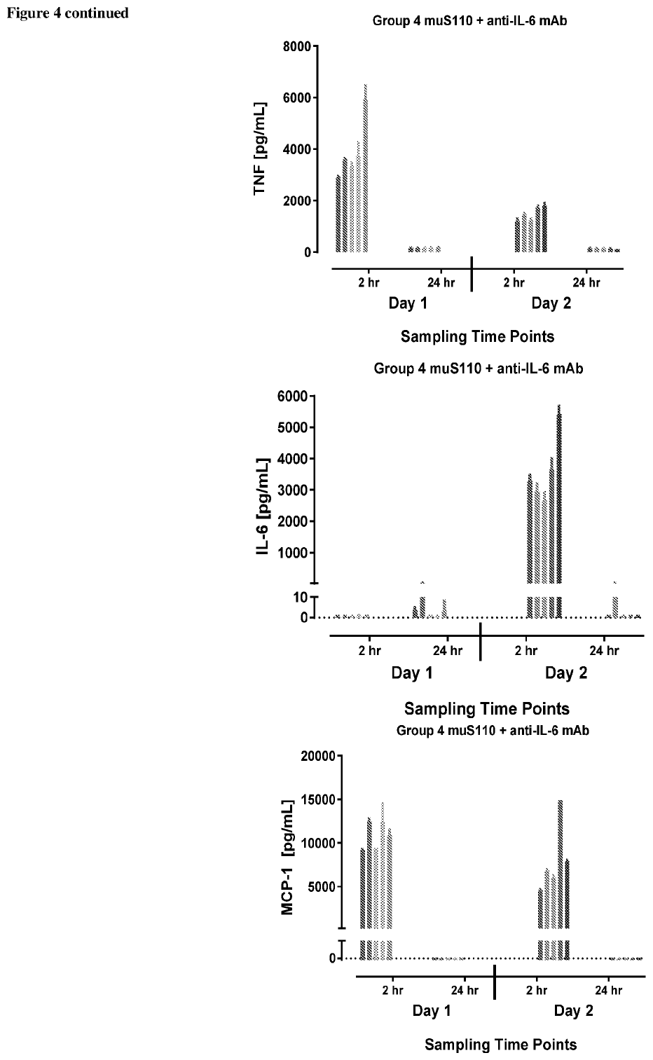

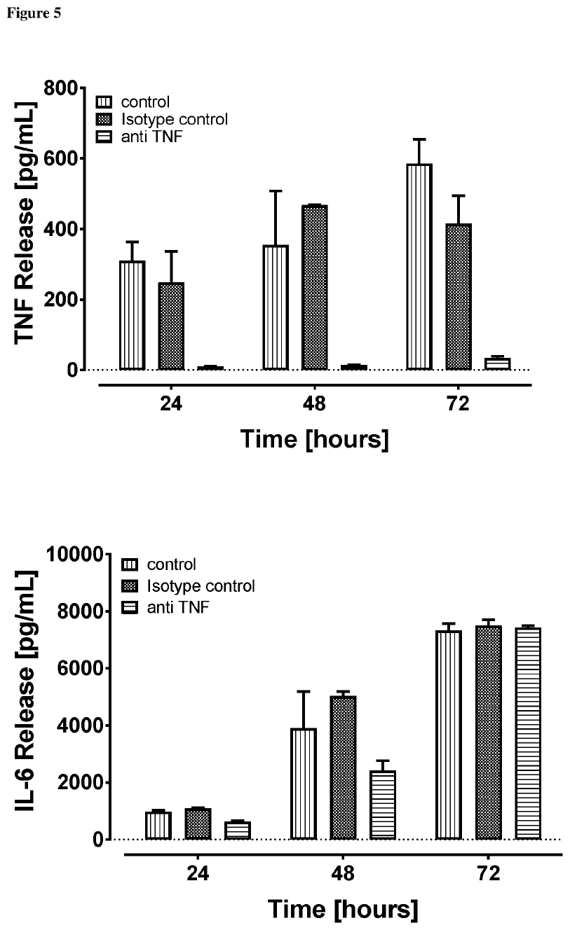

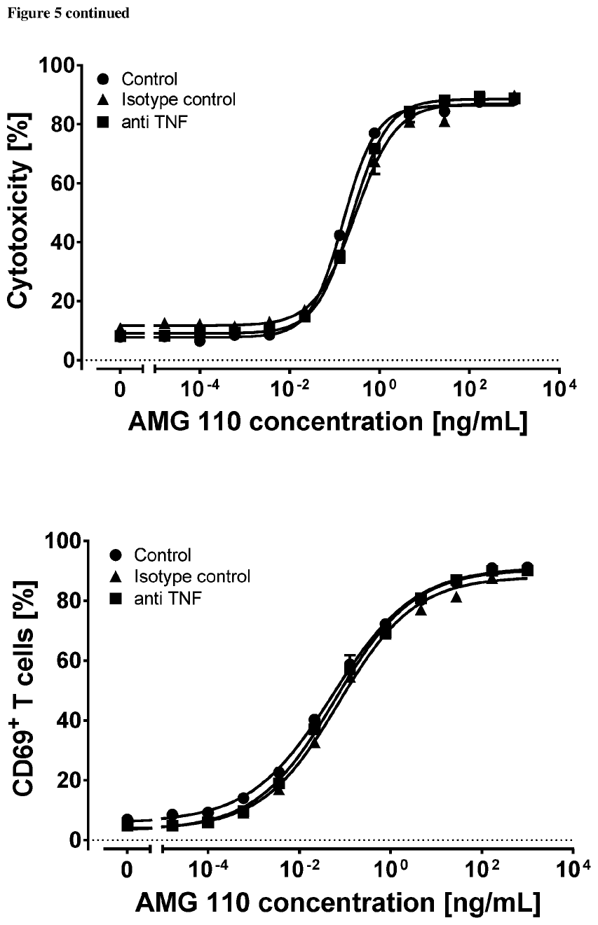

Immunocompetent C57BL/6 mice engineered to express human CD3 (huCD3 knock-in) were pretreated with either a control antibody or two different TNFα blocking agents, an anti-TNFα blocking antibody (anti-TNFα Ab) or a tumor necrosis factor receptor II-Fc fusion protein (TNFRII-Fc). These agents were administered once per day for two days prior to dosing with either an anti-mouse CD19 BiTE® molecule (100 μg/kg or 1000 μg/kg) that recognizes CD19 on B cells, or a negative control BiTE® molecule (n=5 animals per group). Serum cytokines were measured four hours after BiTE® treatment and B cell depletion was measured at 72 hours after BiTE® treatment. The engagement of T cells in vivo using the anti-mouse CD19-targeted BiTE® molecule resulted in activation of T cells, production of cytokines that could be measured in the serum, and depletion of B cells in the spleen. Administration of TNFα blocking reagents prior to BiTE® administration did not affect the ability of the BiTE® molecule to deplete B cells in the spleen (FIG. 1), nor did it impair the release of IFN-γ (FIG. 2), which is important for continued to T cell cytotoxicity, or IL-2 (FIG. 2), which is important for T cell proliferation. However, TNFα blockade did decrease the expression of IL-6 in the serum of animals post-BiTE® administration (FIG. 3). These results show that TNFα blockade can reduce circulating IL-6, which has been implicated in immunotherapy-induced CRS, without affecting BiTE®-mediated cytotoxicity.

### Cytokine Measurements

Milliplex Mouse Cytokine/Chemokine kits (EMD Millipore) were used on Curiox 96w DropArray microplates per both manufacturers' protocols to test the cytokine levels in the original samples collected. Assays were performed such that contents of one EMD Millipore Milliplex kit was used in smaller amounts on 4 Curiox microplates. The lots of the Curiox plates and all items included in the Millipore kits were as shown in Table 1 below.

TABLE 1

| Description | Cat# | Kit lot# | Exp date |
|---|---|---|---|
| Pre-mixed blend | MCYPMX32 | 3136598 | Nov-20 |
| of analytes | | | |
| Standard | MXM8070-2 | SC7N807-8K | May-20 |
| QC1 | MXM6070-2 | MCY-108 | May-20 |
| QC2 | MXM6070-2 | MCY-208 | May-20 |
| Serum Matrix | MXMSM | 3075213 | May-20 |
| Detection | MXM1070-3 | 3207309 | May-21 |
| Antibodies | | | |
| Streptavidin- | L-SAPE10 | 3216830 | Apr-21 |
| Phycoerythrin | | | |
| Assay Buffer | L-AB | 3231126 | May-21 |
| Curiox 96w | #96-CC-BD-05 | C96C2219135-B | May-20 |
| dropArray plates | | | |
| Curiox Wash | — | 1x PBS, 0.1% BSA, | |
| Buffer | | 0.05% Tween20 | |
| | | (PBS 14190-136, | |
| | | lot 17765965) | |

### Preparation of Curiox Items

All samples were run on four Curiox microplates.
Two Curiox Humid Boxes were prepared with 8 paper towels and 100 mL PBS.
Lid sponges on each of two Curiox plates were wetted with 12 mL PBS.
Plates were blocked with 20 μL/well 1% BSA/PBS
Plates were left inside humid box on a Titramax 1000 Shaker (Heidolph Instruments) at ˜400 rpm for 30 minutes

### Preparation of Milliplex Reagents

Beads:

A premix of beads was used in this assay. Analytes IFNγ, IL-2, IL-6 and TNFα were read by the software and subsequently analyzed.

Quality Controls:

One vial of each Quality Control 1 and 2 was reconstituted with 250 μL ddH2O per vial, vortexed lightly (5 seconds), and allowed to sit at room temp for at least 10 minutes before being used.

Wash Buffer:

Curiox Wash Buffer (1×PBS, 0.1% BSA, 0.05% Tween20) was created by mixing. Each liter contains:
900 mL 1×PBS
100 mL 1% BSA in PBS
500 μL 10% Tween20 in PBS

Serum Matrix:

One vial of lyophilized serum matrix was freshly reconstituted with 2 mL Assay Buffer, left to sit at room temp for 10 minutes and then used according to protocol 5 μL per well in standards, quality control, and blank wells.

Serum Dilutions:

Each serum sample was assessed in duplicate and neat only with no dilutions.

Standard Reconstitution and Dilution:

One vial of cytokine standard was freshly reconstituted with 250 μL ddH2O, vortexed lightly (5 seconds), and allowed to sit at room temperature for at least 10 minutes before being diluted and readied for the assay. Standard dilutions were then made at 1:2 such that 100 μL of higher concentrated standard was serially diluted into 100 μL assay buffer to create 13 total dilutions; highest concentration being 10,000 pg/mL, and lowest concentration at 2.4 pg/mL, for all analytes.

### Immunoassay Procedure

The Curiox plates (currently in Block) were each washed 1× using the Curiox DropArray plate washer. Using reverse pipetting (for more accurate dispensing of pipet contents), the following were added to the plates using the plate layouts captured in ELN 20191022-00064. 5 μL bead mix was added per well to all wells. 5 μL Serum Matrix was added to Standards and Quality Control wells. 5 μL Assay Buffer was added to the sample wells. 5 μL Standards and QC controls were added to the appropriate wells. 5 μL sample was added to the appropriate wells. The plates were each mixed 10 seconds using a Vortex Genie plate mixer at 1000 rpm and then left to incubate in the Humid Boxes on a Titramax Shaker, with the magnet array, at ˜400 rpm overnight at 4° C. The following day, plates were washed 3× using the Curiox DropArray plate washer. 5 μL of biotinylated Detection Antibody was added to each well, plates were mixed 10 seconds using a Vortex Genie plate mixer at 1000 rpm, and then were left to incubate in the Humid Boxes, without magnet array, on a Titramax Shaker at ˜400 rpm for 1 hour at room temperature. 5 μL of Streptavidin-PE-labeled detection antibody was added to each well, plates were mixed 10 seconds using a Vortex Genie plate mixer at 1000 rpm and then were left to incubate in the Humid Boxes, without the magnet array, at ˜400 rpm shaking for 30 minutes at room temperature. The plates were again washed 3× using the Curiox DropArray plate washer. 20 μL of sheath fluid was added to each of the wells, plates were mixed 10 seconds using a Vortex Genie plate mixer at 1000 rpm and then were left to incubate in the Humid Boxes, without the magnet array, on a Titramax Shaker at ˜400 rpm for at least 5 minutes at room temperature. Individual well contents from each of the Curiox microplates were then mixed by pipetting up and down at least 10 times and transferred to the wells in a quadrant of a 384-well plate. After all Curiox plates' contents were transferred, 50 μL/well sheath fluid was added to each 384-well plate (now 70 μL/well), and the 384-well plates were mixed 30 seconds using a Vortex Genie plate mixer at 1000 rpm. The 384-well plate was read on a Luminex FlexMap 3D reader using xPONENT software.

### Spleen Flow Cytometry Analysis

At the end of the study, animals were euthanized by carbon dioxide asphyxiation followed by physical cervical dislocation. Spleens were removed and collected in RPMI into individual wells of a 24-well plate. Spleens were pulverized through a 100 μm filter, rinsed with PBS, and 100 μL counting beads (Invitrogen, cat: 01-2222-42, lot:2037700, Carlsbad, Calif.) were added. The disaggregated spleens were then centrifuged at 500 rpm for 5 minutes at 4° C. After decanting the supernatant, the cell pellet was lysed with 1 mL RBC lysis buffer (Unity Lab Services, Amgen, South San Francisco) and quenched with FACS buffer containing FBS (2%). Cells were centrifuged a second time at 500 rpm for 5 minutes at 4° C. After decanting the supernatant, the cell pellet was resuspended in 1 mL FBS-free media. 200 μL of cell suspension was transferred to a 96-well V-bottom plate, live/dead dye exclusion (Invitrogen, Eugene, Oreg.) was added (at a 1:500 dilution) and the cells were incubated for 45 minutes at room temperature in the dark. Cells were washed, centrifuged as previously, and resuspended in 50 μL of Fc block (BD Biosciences, San Jose, Calif.) prior to staining with the following antibodies: BUV395 anti-mouse CD4 clone GK1.5, BV650 anti-mouse CD44 clone IM7, BV421 anti-mouse CD8α clone 53-6.7, PE anti-mouse CD25 clone PC61, BV711 anti-mouse CD62L clone MEL-14 (all from BD Biosciences, San Jose, Calif.), BV510 anti-mouse CD45 clone 30-F11, BV605 anti-mouse Thy1.2 clone 53-2.1, FITC anti-mouse CD19 clone 1D3/CD19, APC anti-mouse CD20 clone SA275A11, AF700 anti-mouse CD69 clone H1.2F3 (all from Biolegend, San Diego, Calif.) and Pe-Cy7 anti-mouse PD1 clone J43 (Thermo Fisher, San Diego, Calif.). Cells were washed and resuspended prior to analysis on a FACSymphony flow cytometer (BD Biosciences, San Jose, Calif.).

### Statistical Analysis

Graphs were generated, and data analysis was performed using GraphPad Prism software (version 7.04, Lo Jolla, Calif.). Mean serum cytokine levels and B cell counts (n=5 animals per group) were expressed as the mean±standard deviation. The p value of 0.05 was used to determine significant differences between all groups using a one-way ANOVA with Tukey's test (GraphPad Prism).

### Example 2

Female BALB/cJ Rj mice (n=10/group) were treated with either vehicle or the BiTE® antibody construct muS110, directed against murine Epithelial Cell Adhesion Molecule for 2 days (Table 2) Animals in group 3 were pre-treated with etanercept (Enbrel) on days −2 and −1 as well as 1 hour before each muS110 administration. Animals in group 4 were administered an anti-IL6 monoclonal antibody (MP5-20F3, BD) on day −1 and one hour before each muS110 treatment. Blood samples were collected 4 and 24 hours after each vehicle and muS110 administration and serum concentrations of IL-6, TNF and MCP1 were determined using the BD™ CBA Mouse Enhanced Sensitivity Flex Set System (Becton Dickinson, BD) in accordance to the manufacturer's instructions (Table 2).

TABLE 2
Study Design

| | | | Route of | Treatment |
|---|---|---|---|---|
| Group | Compound | Dosages | Administration | Schedule* |
| 1 | Vehicle | — | i.v. | Days: 1, 2 |
| 2 | muS110 | 0.05 | i.v. | Days: 1, 2 |
| | | mg/kg/d | | |
| 3 | Enbrel + | Enbrel: | Enbrel: i.p. | Enbrel: |
| | muS110 | 10 mg/kg/d | muS110: i.v. | days −2, −1, 1, 2 |
| | | muS110: | | muS110: |
| | | 0.05 mg/kg/d | | days 1, 2 |
| 4 | Anti-IL6 + | Anti-IL6: | mAb: i.v. | Anti-IL6: |
| | muS110 | 2.5 mg/kg/d | muS110: i.v. | days −1, 1 |
| | | muS110: | | muS110: |
| | | 0.05 mg/kg/d | | days 1, 2 |

*Etanercept (Enbrel) and the anti-IL6 monoclonal antibody were administered 1 hour before muS110 treatment on days 1 and 2.

### In Vitro Anti-TNF Assay

Cytotoxicity, T-cell activation (CD69 upregulation) and cytokine release were determined in multiparametric cytotoxicity assays. Redirected T cell cytotoxicity was evaluated by flow cytometry using isolated human CD3+ T cells as effector cells and Epithelial Cell Adhesion Molecule (EpCAM)-expressing KatoIII tumor cells as target cells. Effector and target cells were co-cultured at an E:T cell ratio of 10:1 and serial dilutions of BiTE® antibody construct AMG110, directed against murine Epithelial Cell Adhesion Molecule in the absence or presence of an anti-TNF monoclonal antibody (BD) or the respective isotype control. AMG 110-mediated redirected target cell lysis was determined after 24, 48 and 72 hours. TNF and IL-6 concentrations in the cell culture supernatant were measured with the CBA human TH1/TH2 Kit in accordance to the manufacturer's instruction.

### BiTE® Molecule Induced MCP-1 Release Blocked by Etanercept (Enbrel)

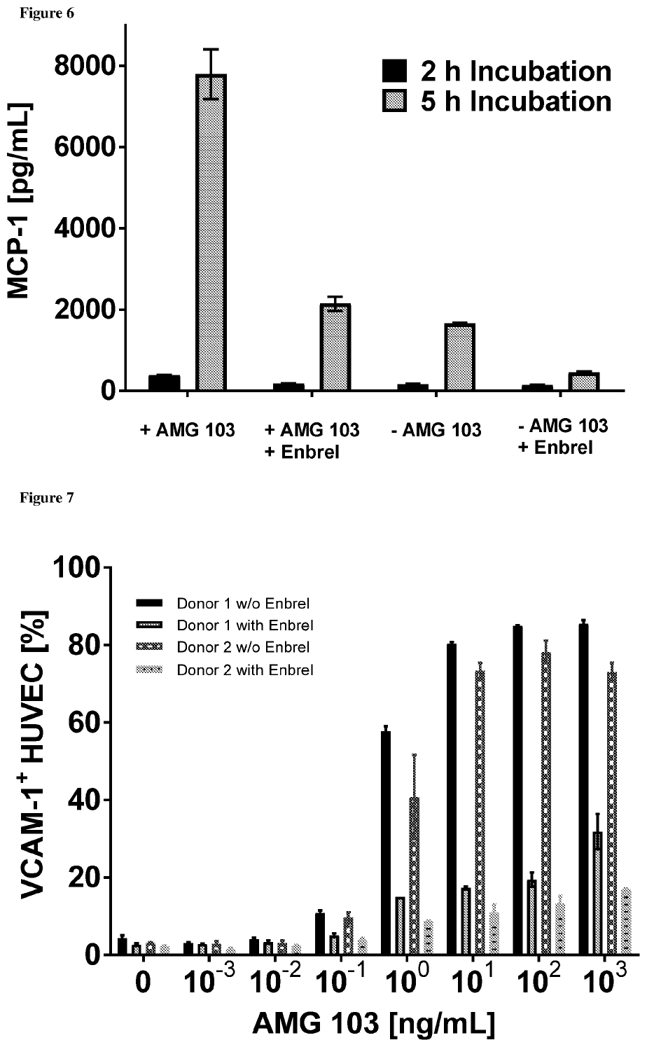

Human umbilical vein endothelial cells (HUVEC) were co-cultured with CD19-expressing NALM-6 cells and human PBMC and increasing concentrations of BiTE® antibody construct blinatumomab (AMG 103) against CD19 in the absence or presence of etanercept. MCP-1 concentrations were determined with the BD Cytometric CBA human MCP-1 Flex set system (BD, Germany after 2 and 5 hours, respectively according to the manufacturer's instructions and are shown in FIG. 6.

### Blinatumumab VCAM Assay

The effect of blinatumomab (AMG 103) on T cell activation and the induction of VCAM-1 on endothelial cells was analyzed in co-cultures of HUVEC, isolated T cells and CD19-expressing NALM-6 target cells. VCAM-1 expression on CD31+HUVEC was analyzed by flow cytometry. The T cell activation marker VCAM-1 was up-regulated by AMG 103, which was sensitive to etanercept, indicating the potential role of TNF in BiTE® molecule-mediated endothelial activation (FIG. 7).

### Example 3

### Etanercept Prophylaxis for PSMA-Targeting BiTE-Related CRS in Humans

Etanercept premedication occurred within two days before BiTE administration to assess whether prophylaxis with the TNF-α inhibitor decreases the frequency and severity of CRS without compromising the efficacy of the BiTE. In an initial cohort of 6 to 8 subjects, the PSMA-targeting BiTE is administered. In addition to dexamethasone prophylaxis, subjects in the cohort received prophylaxis with etanercept (50 mg subcutaneously [SC]) two days prior to each dose of PSMA-targeting BiTE in the first administration cycle. This dose and timing was selected based on the PK properties of etanercept (slow absorption with mean±SD time of maximum concentration [tmax] of 69±34 hours; half-life of 102±30 hours) (Enbrel Package Insert, 2017; Zhou et al., 2011).

Subject incidence of safety events, including dose-limiting toxicities (DLTs), serious adverse events (SAEs), and adverse events leading to treatment discontinuation or dose reduction, will be reviewed when at least 3 DLT-evaluable subjects complete the DLT window. Subjects receiving three individual increasing doses of PSMA-targeting BiTE with etanercept prophylaxis experienced 1 DLT/SAE (grade 3 (G3) thrombocytopenia), 1 SAE (Grade 3 (G3) Afib), 0 dose reductions, 1 discontinuation (tolerability), CRS events in cycle 1: 4 grade 2 (G2), no grade 3/4 (G3/4). Etanercept prophylaxis improves CRS safety profile compared with no premedication or premedication with tocilizumab (8 mg/kg) given two hours before administration of the first dose of the PSMA-targeting BiTE. In the latter group 1 DLT/SAE (G3 Hypoacusis), 2 SAE (grade 3 (G3) Pruritus, grade 2/3 (G2/G3) CRS), 2 dose reductions, 2 discontinuations (disease progression), and CRS events in cycle 1: 4 grade 1 (G1), 7 grade 2 (G2), 4 grade 3 (G3) were observed. In the etanercept prophylaxis group the PSMA-targeting BiTE was administered at the same dosages and frequency as in the group without etanercept prophylaxis.

TABLE 3
Sequences

| SEQ | | | |
|---|---|---|---|
| ID | | Format/ | |
| NO | Designation | Source | Amino acid sequence |
| 1 | CD3ϵ ECD | human | QDGNEEMGGITQTPYKVSISGTTVILTCPQYPGSEILWQHNDKNIGGDEDDKNIGSDEDHLSLK |
| | | | EFSELEQSGYYVCYPRGSKPEDANFYLYLRARVCENCMEMD |
| 2 | CD3ϵ ECD | human | QDGNEEMGGITQTPYKVSISGTTVILT |
| | pos. 1-27 | | |
| 3 | CDR-L1 of H2C | artificial | GSSTGAVTSGYYPN |
| 4 | CDR-L2 of H2C | artificial | GTKFLAP |
| 5 | CDR-L3 of H2C | artificial | ALWYSNRWV |
| 6 | CDR-L1 of E2M | artificial | RSSTGAVTSGYYPN |
| 7 | CDR-L2 of E2M | artificial | ATDMRPS |
| 8 | CDR-L3 of E2M | artificial | ALWYSNRWV |
| 9 | CDR-L1 of F12Q | artificial | GSSTGAVTSGNYPN |
| 10 | CDR-L2 of F12Q | artificial | GTKFLAP |
| 11 | CDR-L3 of F12Q | artificial | VLWYSNRWV |
| 12 | CDR-H1 of F6A | artificial | IYAMN |
| 13 | CDR-H2 of F6A | artificial | RIRSKYNNYATYYADSVKS |
| 14 | CDR-H3 of F6A | artificial | HGNFGNSYVSFFAY |
| 15 | CDR-H1 of H2C | artificial | KYAMN |
| 16 | CDR-H2 of H2C | artificial | RIRSKYNNYATYYADSVKD |
| 17 | CDR-H3 of H2C | artificial | HGNFGNSYISYWAY |
| 18 | CDR-H1 of H1E | artificial | SYAMN |
| 19 | CDR-H2 of H1E | artificial | RIRSKYNNYATYYADSVKG |
| 20 | CDR-H3 of H1E | artificial | HGNFGNSYLSFWAY |
| 21 | CDR-H1 of G4H | artificial | RYAMN |
| 22 | CDR-H2 of G4H | artificial | RIRSKYNNYATYYADSVKG |
| 23 | CDR-H3 of G4H | artificial | HGNFGNSYLSYFAY |
| 24 | CDR-H1 of A2J | artificial | VYAMN |
| 25 | CDR-H2 of A2J | artificial | RIRSKYNNYATYYADSVKK |
| 26 | CDR-H3 of A2J | artificial | HGNFGNSYLSWWAY |
| 27 | CDR-H1 of ElL | artificial | KYAMN |
| 28 | CDR-H2 of ElL | artificial | RIRSKYNNYATYYADSVKS |
| 29 | CDR-H3 of ElL | artificial | HGNFGNSYTSYYAY |
| 30 | CDR-H1 of E2M | artificial | GYAMN |
| 31 | CDR-H2 of E2M | artificial | RIRSKYNNYATYYADSVKE |
| 32 | CDR-H3 of E2M | artificial | HRNFGNSYLSWFAY |
| 33 | CDR-H1 of F7O | artificial | VYAMN |
| 34 | CDR-H2 of F7O | artificial | RIRSKYNNYATYYADSVKK |
| 35 | CDR-H3 of F7O | artificial | HGNFGNSYISWWAY |
| 36 | CDR-H1 of F12Q | artificial | SYAMN |
| 37 | CDR-H2 of F12Q | artificial | RIRSKYNNYATYYADSVKG |
| 38 | CDR-H3 of F12Q | artificial | HGNFGNSYVSWWAY |
| 39 | CDR-H1 of I2C | artificial | KYAMN |
| 40 | CDR-H2 of I2C | artificial | RIRSKYNNYATYYADSVKD |
| 41 | CDR-H3 of I2C | artificial | HGNFGNSYISYWAY |
| 42 | CDR-L2 of H2C | artificial | GTKFLAP |
| 43 | CDR-L3 of H2C | artificial | ALWYSNRWV |
| 44 | CDR-H1 of H2C | artificial | KYAMN |
| 45 | CDR-L1 of H1E | artificial | GSSTGAVTSGYYPN |
| 46 | CDR-L2 of H1E | artificial | GTKFLAP |
| 47 | CDR-L3 of H1E | artificial | ALWYSNRWV |
| 48 | CDR-H1 of H1E | artificial | SYAMN |
| 49 | CDR-H2 of H1E | artificial | RIRSKYNNYATYYADSVKG |
| 50 | CDR-H3 of H1E | artificial | HGNFGNSYLSFWAY |
| 51 | CDR-L1 of G4H | artificial | GSSTGAVTSGYYPN |
| 52 | CDR-L2 of G4H | artificial | GTKFLAP |
| 53 | CDR-L3 of G4H | artificial | ALWYSNRWV |
| 54 | CDR-L1 of A2J | artificial | RSSTGAVTSGYYPN |
| 55 | CDR-L2 of A2J | artificial | ATDMRPS |
| 56 | CDR-L3 of A2J | artificial | ALWYSNRWV |
| 57 | CDR-L1 of ElL | artificial | GSSTGAVTSGYYPN |
| 58 | CDR-L2 of ElL | artificial | GTKFLAP |
| 59 | CDR-L3 of ElL | artificial | ALWYSNRWV |
| 60 | CDR-L1 of F7O | artificial | GSSTGAVTSGYYPN |
| 61 | CDR-L2 of F7O | artificial | GTKFLAP |
| 62 | CDR-L3 of F7O | artificial | ALWYSNRWV |
| 63 | CDR-L1 of I2C | artificial | GSSTGAVTSGNYPN |
| 64 | CDR-L2 of I2C | artificial | GTKFLAP |
| 65 | CDR-L3 of I2C | artificial | VLWYSNRWV |
| 66 | VL of H2C | artificial | QTVVTQEPSLTVSPGGTVTLTCGSSTGAVTSGYYPNWVQQKPGQAPRGLIGGTKFLAPGTPARF |
| | | | SGSLLGGKAALTLSGVQPEDEAEYYCALWYSNRWVFGGGTKLTVL |
| 67 | VL of E2M | artificial | QTVVTQEPSLTVSPGGTVTLTCRSSTGAVTSGYYPNWVQQKPGQAPRGLIGATDMRPSGTPARF |
| | | | SGSLLGGKAALTLSGVQPEDEAEYYCALWYSNRWVFGGGTKLTVL |
| 68 | VL of F12Q | artificial | QTVVTQEPSLTVSPGGTVTLTCGSSTGAVTSGNYPNWVQQKPGQAPRGLIGGTKFLAPGTPARF |
| | | | SGSLLGGKAALTLSGVQPEDEAEYYCVLWYSNRWVFGGGTKLTVL |
| 69 | VL variant | artificial | ELVVTQEPSLTVSPGGTVTLTCGSSTGAVTSGYYPNWVQQKPGQAPRGLIGGTKFLAPGTPARF |
| | of H2C | | SGSLLGGKAALTLSGVQPEDEAEYYCALWYSNRWVFGGGTKLTVL |
| 70 | VL variant | artificial | ELVVTQEPSLTVSPGGTVTLTCRSSTGAVTSGYYPNWVQQKPGQAPRGLIGATDMRPSGTPARF |
| | of A2J | | SGSLLGGKAALTLSGVQPEDEAEYYCALWYSNRWVFGGGTKLTVL |
| 71 | VL variant | artificial | ELVVTQEPSLTVSPGGTVTLTCGSSTGAVTSGNYPNWVQQKPGQAPRGLIGGTKFLAPGTPARF |
| | of F12Q | | SGSLLGGKAALTLSGVQPEDEAEYYCVLWYSNRWVFGGGTKLTVL |
| 72 | VH of F6A | artificial | EVQLVESGGGLVQPGGSLKLSCAASGFTFNIYAMNWVRQAPGKGLEWVARIRSKYNNYATYYAD |
| | | | SVKSRFTISRDDSKNTAYLQMNNLKTEDTAVYYCVRHGNFGNSYVSFFAYWGQGTLVTVSS |
| 73 | VH of H2C | artificial | EVQLVESGGGLVQPGGSLKLSCAASGFTFNKYAMNWVRQAPGKGLEWVARIRSKYNNYATYYAD |
| | | | SVKDRFTISRDDSKNTAYLQMNNLKTEDTAVYYCVRHGNFGNSYISYWAYWGQGTLVTVSS |
| 74 | VH of H1E | artificial | EVQLVESGGGLEQPGGSLKLSCAASGFTFNSYAMNWVRQAPGKGLEWVARIRSKYNNYATYYAD |
| | | | SVKGRFTISRDDSKNTAYLQMNNLKTEDTAVYYCVRHGNFGNSYLSFWAYWGQGTLVTVSS |
| 75 | VH of G4H | artificial | EVQLVESGGGLVQPGGSLKLSCAASGFTFNRYAMNWVRQAPGKGLEWVARIRSKYNNYATYYAD |
| | | | SVKGRFTISRDDSKNTAYLQMNNLKTEDTAVYYCVRHGNFGNSYLSYFAYWGQGTLVTVSS |
| 76 | VH of A2J | artificial | EVQLVESGGGLVQPGGSLKLSCAASGFTFNVYAMNWVRQAPGKGLEWVARIRSKYNNYATYYAD |
| | | | SVKKRFTISRDDSKNTAYLQMNNLKTEDTAVYYCVRHGNFGNSYLSWWAYWGQGTLVTVSS |
| 77 | VH of ElL | artificial | EVQLVESGGGLVQPGGSLKLSCAASGFTFNKYAMNWVRQAPGKGLEWVARIRSKYNNYATYYAD |
| | | | SVKSRFTISRDDSKNTAYLQMNNLKTEDTAVYYCVRHGNFGNSYTSYYAYWGQGTLVTVSS |
| 78 | VH of E2M | artificial | EVQLVESGGGLVQPGGSLKLSCAASGFTFNGYAMNWVRQAPGKGLEWVARIRSKYNNYATYYAD |
| | | | SVKERFTISRDDSKNTAYLQMNNLKTEDTAVYYCVRHRNFGNSYLSWFAYWGQGTLVTVSS |
| 79 | VH of F7O | artificial | EVQLVESGGGLVQPGGSLKLSCAASGFTFNVYAMNWVRQAPGKGLEWVARIRSKYNNYATYYAD |
| | | | SVKKRFTISRDDSKNTAYLQMNNLKTEDTAVYYCVRHGNFGNSYISWWAYWGQGTLVTVSS |
| 80 | VH of F12Q | artificial | EVQLVESGGGLVQPGGSLKLSCAASGFTFNSYAMNWVRQAPGKGLEWVARIRSKYNNYATYYAD |
| | | | SVKGRFTISRDDSKNTAYLQMNNLKTEDTAVYYCVRHGNFGNSYVSWWAYWGQGTLVTVSS |
| 81 | VH of I2C | artificial | EVQLVESGGGLVQPGGSLKLSCAASGFTFNKYAMNWVRQAPGKGLEWVARIRSKYNNYATYYAD |
| | | | SVKDRFTISRDDSKNTAYLQMNNLKTEDTAVYYCVRHGNFGNSYISYWAYWGQGTLVTVSS |
| 82 | VH of F12q | artificial | EVQLVESGGGLVQPGGSLRLSCAASGFTFNSYAMNWVRQAPGKGLEWVARIRSKYNNYATYYAD |
| | | | SVKGRFTISRDDSKNTAYLQMNSLKTEDTAVYYCVRHGNFGNSYVSWWAYWGQGTLVTVSS |
| 83 | VH variant of F6A | artificial | EVQLLESGGGLVQPGGSLKLSCAASGFTFNIYAMNWVRQAPGKGLEWVARIRSKYNNYATYYAD |
| | | | SVKSRFTISRDDSKNTAYLQMNNLKTEDTAVYYCVRHGNFGNSYVSFFAYWGQGTLVTVSS |
| 84 | VH variant of H2C | artificial | EVQLLESGGGLVQPGGSLKLSCAASGFTFNKYAMNWVRQAPGKGLEWVARIRSKYNNYATYYAD |
| | | | SVKDRFTISRDDSKNTAYLQMNNLKTEDTAVYYCVRHGNFGNSYISYWAYWGQGTLVTVSS |
| 85 | VH variant of H1E | artificial | EVQLLESGGGLEQPGGSLKLSCAASGFTFNSYAMNWVRQAPGKGLEWVARIRSKYNNYATYYAD |
| | | | SVKGRFTISRDDSKNTAYLQMNNLKTEDTAVYYCVRHGNFGNSYLSFWAYWGQGTLVTVSS |
| 86 | VH variant of G4H | artificial | EVQLLESGGGLVQPGGSLKLSCAASGFTFNRYAMNWVRQAPGKGLEWVARIRSKYNNYATYYAD |
| | | | SVKGRFTISRDDSKNTAYLQMNNLKTEDTAVYYCVRHGNFGNSYLSYFAYWGQGTLVTVSS |
| 87 | VH variant of A2J | artificial | EVQLLESGGGLVQPGGSLKLSCAASGFTFNVYAMNWVRQAPGKGLEWVARIRSKYNNYATYYAD |
| | | | SVKKRFTISRDDSKNTAYLQMNNLKTEDTAVYYCVRHGNFGNSYLSWWAYWGQGTLVTVSS |
| 88 | VH variant of E1L | artificial | EVQLLESGGGLVQPGGSLKLSCAASGFTFNKYAMNWVRQAPGKGLEWVARIRSKYNNYATYYAD |
| | | | SVKSRFTISRDDSKNTAYLQMNNLKTEDTAVYYCVRHGNFGNSYTSYYAYWGQGTLVTVSS |
| 89 | VH variant of E2M | artificial | EVQLLESGGGLVQPGGSLKLSCAASGFTFNGYAMNWVRQAPGKGLEWVARIRSKYNNYATYYAD |
| | | | SVKERFTISRDDSKNTAYLQMNNLKTEDTAVYYCVRHRNFGNSYLSWFAYWGQGTLVTVSS |
| 90 | VH variant of F7O | artificial | EVQLLESGGGLVQPGGSLKLSCAASGFTFNVYAMNWVRQAPGKGLEWVARIRSKYNNYATYYAD |
| | | | SVKKRFTISRDDSKNTAYLQMNNLKTEDTAVYYCVRHGNFGNSYISWWAYWGQGTLVTVSS |
| 91 | VH variant of F12Q | artificial | EVQLLESGGGLVQPGGSLKLSCAASGFTFNSYAMNWVRQAPGKGLEWVARIRSKYNNYATYYAD |
| | | | SVKGRFTISRDDSKNTAYLQMNNLKTEDTAVYYCVRHGNFGNSYVSWWAYWGQGTLVTVSS |
| 92 | VH variant of I2C | artificial | EVQLLESGGGLVQPGGSLKLSCAASGFTFNKYAMNWVRQAPGKGLEWVARIRSKYNNYATYYAD |
| | | | SVKDRFTISRDDSKNTAYLQMNNLKTEDTAVYYCVRHGNFGNSYISYWAYWGQGTLVTVSS |
| 93 | VL of F6A | artificial | QTVVTQEPSLTVSPGGTVTLTCGSSTGAVTSGYYPNWVQQKPGQAPRGLIGGTKFLAPGTPARF |
| | | | SGSLLGGKAALTLSGVQPEDEAEYYCALWYSNRWVFGGGTKLTVL |
| 94 | VL of HlE | artificial | QTVVTQEPSLTVSPGGTVTLTCGSSTGAVTSGYYPNWVQQKPGQAPRGLIGGTKFLAPGTPARF |
| | | | SGSLLGGKAALTLSGVQPEDEAEYYCALWYSNRWVFGGGTKLTVL |
| 95 | VL of G4H | artificial | QTVVTQEPSLTVSPGGTVTLTCGSSTGAVTSGYYPNWVQQKPGQAPRGLIGGTKFLAPGTPARF |
| | | | SGSLLGGKAALTLSGVQPEDEAEYYCALWYSNRWVFGGGTKLTVL |
| 96 | VL of A2J | artificial | QTVVTQEPSLTVSPGGTVTLTCRSSTGAVTSGYYPNWVQQKPGQAPRGLIGATDMRPSGTPARF |
| | | | SGSLLGGKAALTLSGVQPEDEAEYYCALWYSNRWVFGGGTKLTVL |
| 97 | VL of E1L | artificial | QTVVTQEPSLTVSPGGTVTLTCGSSTGAVTSGYYPNWVQQKPGQAPRGLIGGTKFLAPGTPARF |
| | | | SGSLLGGKAALTLSGVQPEDEAEYYCALWYSNRWVFGGGTKLTVL |
| 98 | VL of F7O | artificial | QTVVTQEPSLTVSPGGTVTLTCGSSTGAVTSGYYPNWVQQKPGQAPRGLIGGTKFLAPGTPARF |
| | | | SGSLLGGKAALTLSGVQPEDEAEYYCALWYSNRWVFGGGTKLTVL |
| 99 | VL of I2C | artificial | QTVVTQEPSLTVSPGGTVTLTCGSSTGAVTSGNYPNWVQQKPGQAPRGLIGGTKFLAPGTPARF |
| | | | SGSLLGGKAALTLSGVQPEDEAEYYCVLWYSNRWVFGGGTKLTVL |
| 100 | VL of F12q | artificial | QTVVTQEPSLTVSPGGTVTLTCGSSTGAVTSGNYPNWVQQKPGQAPRGLIGGTKFLAPGTPARF |
| | | | SGSLLGGKAALTLSGVQPEDEAEYYCVLWYSNRWVFGGGTKLTVL |
| 101 | scFv of F6A | artificial | EVQLVESGGGLVQPGGSLKLSCAASGFTFNIYAMNWVRQAPGKGLEWVARIRSKYNNYATYYAD |
| | | | SVKSRFTISRDDSKNTAYLQMNNLKTEDTAVYYCVRHGNFGNSYVSFFAYWGQGTLVTVSSGGG |
| | | | GSGGGGSGGGGSQTVVTQEPSLTVSPGGTVTLTCGSSTGAVTSGYYPNWVQQKPGQAPRGLIGG |
| | | | TKFLAPGTPARFSGSLLGGKAALTLSGVQPEDEAEYYCALWYSNRWVFGGGTKLTVL |
| 102 | scFv of H2C | artificial | EVQLVESGGGLVQPGGSLKLSCAASGFTFNKYAMNWVRQAPGKGLEWVARIRSKYNNYATYYAD |
| | | | SVKDRFTISRDDSKNTAYLQMNNLKTEDTAVYYCVRHGNFGNSYISYWAYWGQGTLVTVSSGGG |
| | | | GSGGGGSGGGGSQTVVTQEPSLTVSPGGTVTLTCGSSTGAVTSGYYPNWVQQKPGQAPRGLIGG |
| | | | TKFLAPGTPARFSGSLLGGKAALTLSGVQPEDEAEYYCALWYSNRWVFGGGTKLTVL |
| 103 | scFv of H1E | artificial | EVQLVESGGGLEQPGGSLKLSCAASGFTFNSYAMNWVRQAPGKGLEWVARIRSKYNNYATYYAD |
| | | | SVKGRFTISRDDSKNTAYLQMNNLKTEDTAVYYCVRHGNFGNSYLSFWAYWGQGTLVTVSSGGG |
| | | | GSGGGGSGGGGSQTVVTQEPSLTVSPGGTVTLTCGSSTGAVTSGYYPNWVQQKPGQAPRGLIGG |
| | | | TKFLAPGTPARFSGSLLGGKAALTLSGVQPEDEAEYYCALWYSNRWVFGGGTKLTVL |
| 104 | scFv of G4H | artificial | EVQLVESGGGLVQPGGSLKLSCAASGFTFNRYAMNWVRQAPGKGLEWVARIRSKYNNYATYYAD |
| | | | SVKGRFTISRDDSKNTAYLQMNNLKTEDTAVYYCVRHGNFGNSYLSYFAYWGQGTLVTVSSGGG |
| | | | GSGGGGSGGGGSQTVVTQEPSLTVSPGGTVTLTCGSSTGAVTSGYYPNWVQQKPGQAPRGLIGG |
| | | | TKFLAPGTPARFSGSLLGGKAALTLSGVQPEDEAEYYCALWYSNRWVFGGGTKLTVL |
| 105 | scFv of A2J | artificial | EVQLVESGGGLVQPGGSLKLSCAASGFTFNVYAMNWVRQAPGKGLEWVARIRSKYNNYATYYAD |
| | | | SVKKRFTISRDDSKNTAYLQMNNLKTEDTAVYYCVRHGNFGNSYLSWWAYWGQGTLVTVSSGGG |
| | | | GSGGGGSGGGGSQTVVTQEPSLTVSPGGTVTLTCRSSTGAVTSGYYPNWVQQKPGQAPRGLIGA |
| | | | TDMRPSGTPARFSGSLLGGKAALTLSGVQPEDEAEYYCALWYSNRWVFGGGTKLTVL |
| 106 | scFv of E1L | artificial | EVQLVESGGGLVQPGGSLKLSCAASGFTFNKYAMNWVRQAPGKGLEWVARIRSKYNNYATYYAD |
| | | | SVKSRFTISRDDSKNTAYLQMNNLKTEDTAVYYCVRHGNFGNSYTSYYAYWGQGTLVTVSSGGG |
| | | | GSGGGGSGGGGSQTVVTQEPSLTVSPGGTVTLTCGSSTGAVTSGYYPNWVQQKPGQAPRGLIGG |
| | | | TKFLAPGTPARFSGSLLGGKAALTLSGVQPEDEAEYYCALWYSNRWVFGGGTKLTVL |
| 107 | scFv of E2M | artificial | EVQLVESGGGLVQPGGSLKLSCAASGFTFNGYAMNWVRQAPGKGLEWVARIRSKYNNYATYYAD |
| | | | SVKERFTISRDDSKNTAYLQMNNLKTEDTAVYYCVRHRNFGNSYLSWFAYWGQGTLVTVSSGGG |
| | | | GSGGGGSGGGGSQTVVTQEPSLTVSPGGTVTLTCRSSTGAVTSGYYPNWVQQKPGQAPRGLIGA |
| | | | TDMRPSGTPARFSGSLLGGKAALTLSGVQPEDEAEYYCALWYSNRWVFGGGTKLTVL |
| 108 | scFv of F7O | artificial | EVQLVESGGGLVQPGGSLKLSCAASGFTFNVYAMNWVRQAPGKGLEWVARIRSKYNNYATYYAD |
| | | | SVKKRFTISRDDSKNTAYLQMNNLKTEDTAVYYCVRHGNFGNSYISWWAYWGQGTLVTVSSGGG |
| | | | GSGGGGSGGGGSQTVVTQEPSLTVSPGGTVTLTCGSSTGAVTSGYYPNWVQQKPGQAPRGLIGG |
| | | | TKFLAPGTPARFSGSLLGGKAALTLSGVQPEDEAEYYCALWYSNRWVFGGGTKLTVL |
| 109 | scFv of F12Q | artificial | EVQLVESGGGLVQPGGSLKLSCAASGFTFNSYAMNWVRQAPGKGLEWVARIRSKYNNYATYYAD |
| | | | SVKGRFTISRDDSKNTAYLQMNNLKTEDTAVYYCVRHGNFGNSYVSWWAYWGQGTLVTVSSGGG |
| | | | GSGGGGSGGGGSQTVVTQEPSLTVSPGGTVTLTCGSSTGAVTSGNYPNWVQQKPGQAPRGLIGG |
| | | | TKFLAPGTPARFSGSLLGGKAALTLSGVQPEDEAEYYCVLWYSNRWVFGGGTKLTVL |
| 110 | scFv of I2C | artificial | EVQLVESGGGLVQPGGSLKLSCAASGFTFNKYAMNWVRQAPGKGLEWVARIRSKYNNYATYYAD |
| | | | SVKDRFTISRDDSKNTAYLQMNNLKTEDTAVYYCVRHGNFGNSYISYWAYWGQGTLVTVSSGGG |
| | | | GSGGGGSGGGGSQTVVTQEPSLTVSPGGTVTLTCGSSTGAVTSGNYPNWVQQKPGQAPRGLIGG |
| | | | TKFLAPGTPARFSGSLLGGKAALTLSGVQPEDEAEYYCVLWYSNRWVFGGGTKLTVL |
| ill | scFv of F12q | artificial | EVQLVESGGGLVQPGGSLRLSCAASGFTFNSYAMNWVRQAPGKGLEWVARIRSKYNNYATYYAD |
| | | | SVKGRFTISRDDSKNTAYLQMNSLKTEDTAVYYCVRHGNFGNSYVSWWAYWGQGTLVTVSSGGG |
| | | | GSGGGGSGGGGSQTVVTQEPSLTVSPGGTVTLTCGSSTGAVTSGNYPNWVQQKPGQAPRGLIGG |
| | | | TKFLAPGTPARFSGSLLGGKAALTLSGVQPEDEAEYYCVLWYSNRWVFGGGTKLTVL |
| 112 | scFv variant | artificial | EVQLLESGGGLVQPGGSLKLSCAASGFTFNIYAMNWVRQAPGKGLEWVARIRSKYNNYATYYAD |
| | of F6A | | SVKSRFTISRDDSKNTAYLQMNNLKTEDTAVYYCVRHGNFGNSYVSFFAYWGQGTLVTVSSGGG |
| | | | GSGGGGSGGGGSELVVTQEPSLTVSPGGTVTLTCGSSTGAVTSGYYPNWVQQKPGQAPRGLIGG |
| | | | TKFLAPGTPARFSGSLLGGKAALTLSGVQPEDEAEYYCALWYSNRWVFGGGTKLTVL |
| 113 | scFv variant | artificial | EVQLLESGGGLVQPGGSLKLSCAASGFTFNKYAMNWVRQAPGKGLEWVARIRSKYNNYATYYAD |
| | of H2C | | SVKDRFTISRDDSKNTAYLQMNNLKTEDTAVYYCVRHGNFGNSYISYWAYWGQGTLVTVSSGGG |
| | | | GSGGGGSGGGGSELVVTQEPSLTVSPGGTVTLTCGSSTGAVTSGYYPNWVQQKPGQAPRGLIGG |
| | | | TKFLAPGTPARFSGSLLGGKAALTLSGVQPEDEAEYYCALWYSNRWVFGGGTKLTVL |
| 114 | scFv variant | artificial | EVQLLESGGGLEQPGGSLKLSCAASGFTFNSYAMNWVRQAPGKGLEWVARIRSKYNNYATYYAD |
| | of H1E | | SVKGRFTISRDDSKNTAYLQMNNLKTEDTAVYYCVRHGNFGNSYLSFWAYWGQGTLVTVSSGGG |
| | | | GSGGGGSGGGGSELVVTQEPSLTVSPGGTVTLTCGSSTGAVTSGYYPNWVQQKPGQAPRGLIGG |
| | | | TKFLAPGTPARFSGSLLGGKAALTLSGVQPEDEAEYYCALWYSNRWVFGGGTKLTVL |
| 115 | scFv variant | artificial | EVQLLESGGGLVQPGGSLKLSCAASGFTFNRYAMNWVRQAPGKGLEWVARIRSKYNNYATYYAD |
| | of G4H | | SVKGRFTISRDDSKNTAYLQMNNLKTEDTAVYYCVRHGNFGNSYLSYFAYWGQGTLVTVSSGGG |
| | | | GSGGGGSGGGGSELVVTQEPSLTVSPGGTVTLTCGSSTGAVTSGYYPNWVQQKPGQAPRGLIGG |
| | | | TKFLAPGTPARFSGSLLGGKAALTLSGVQPEDEAEYYCALWYSNRWVFGGGTKLTVL |
| 116 | scFv variant | artificial | EVQLLESGGGLVQPGGSLKLSCAASGFTFNVYAMNWVRQAPGKGLEWVARIRSKYNNYATYYAD |
| | of A2J | | SVKKRFTISRDDSKNTAYLQMNNLKTEDTAVYYCVRHGNFGNSYLSWWAYWGQGTLVTVSSGGG |
| | | | GSGGGGSGGGGSELVVTQEPSLTVSPGGTVTLTCRSSTGAVTSGYYPNWVQQKPGQAPRGLIGA |
| | | | TDMRPSGTPARFSGSLLGGKAALTLSGVQPEDEAEYYCALWYSNRWVFGGGTKLTVL |
| 117 | scFv variant | artificial | EVQLLESGGGLVQPGGSLKLSCAASGFTFNKYAMNWVRQAPGKGLEWVARIRSKYNNYATYYAD |
| | of E1L | | SVKSRFTISRDDSKNTAYLQMNNLKTEDTAVYYCVRHGNFGNSYTSYYAYWGQGTLVTVSSGGG |
| | | | GSGGGGSGGGGSELVVTQEPSLTVSPGGTVTLTCGSSTGAVTSGYYPNWVQQKPGQAPRGLIGG |
| | | | TKFLAPGTPARFSGSLLGGKAALTLSGVQPEDEAEYYCALWYSNRWVFGGGTKLTVL |
| 118 | scFv variant | artificial | EVQLLESGGGLVQPGGSLKLSCAASGFTFNGYAMNWVRQAPGKGLEWVARIRSKYNNYATYYAD |
| | of E2M | | SVKERFTISRDDSKNTAYLQMNNLKTEDTAVYYCVRHRNFGNSYLSWFAYWGQGTLVTVSSGGG |
| | | | GSGGGGSGGGGSELVVTQEPSLTVSPGGTVTLTCRSSTGAVTSGYYPNWVQQKPGQAPRGLIGA |
| | | | TDMRPSGTPARFSGSLLGGKAALTLSGVQPEDEAEYYCALWYSNRWVFGGGTKLTVL |
| 119 | scFv variant | artificial | EVQLLESGGGLVQPGGSLKLSCAASGFTFNVYAMNWVRQAPGKGLEWVARIRSKYNNYATYYAD |
| | of F7O | | SVKKRFTISRDDSKNTAYLQMNNLKTEDTAVYYCVRHGNFGNSYISWWAYWGQGTLVTVSSGGG |
| | | | GSGGGGSGGGGSELVVTQEPSLTVSPGGTVTLTCGSSTGAVTSGYYPNWVQQKPGQAPRGLIGG |
| | | | TKFLAPGTPARFSGSLLGGKAALTLSGVQPEDEAEYYCALWYSNRWVFGGGTKLTVL |
| 120 | scFv variant | artificial | EVQLLESGGGLVQPGGSLKLSCAASGFTFNSYAMNWVRQAPGKGLEWVARIRSKYNNYATYYAD |
| | of F12Q | | SVKGRFTISRDDSKNTAYLQMNNLKTEDTAVYYCVRHGNFGNSYVSWWAYWGQGTLVTVSSGGG |
| | | | GSGGGGSGGGGSELVVTQEPSLTVSPGGTVTLTCGSSTGAVTSGNYPNWVQQKPGQAPRGLIGG |
| | | | TKFLAPGTPARFSGSLLGGKAALTLSGVQPEDEAEYYCVLWYSNRWVFGGGTKLTVL |
| 121 | scFv variant | artificial | EVQLLESGGGLVQPGGSLKLSCAASGFTFNKYAMNWVRQAPGKGLEWVARIRSKYNNYATYYAD |
| | of I2C | | SVKDRFTISRDDSKNTAYLQMNNLKTEDTAVYYCVRHGNFGNSYISYWAYWGQGTLVTVSSGGG |
| | | | GSGGGGSGGGGSELVVTQEPSLTVSPGGTVTLTCGSSTGAVTSGNYPNWVQQKPGQAPRGLIGG |
| | | | TKFLAPGTPARFSGSLLGGKAALTLSGVQPEDEAEYYCVLWYSNRWVFGGGTKLTVL |
| 122 | 20A2 VH CDR1 | artificial | NYGMH |
| 123 | 20A2 VH CDR2 | artificial | LIWYDGSKKYYADSVKG |
| 124 | 20A2 VH CDR3 | artificial | DPSSLTGSTGYYGMDV |
| 125 | 20A2 VL CDR1 | artificial | SGDKLGDKYAC |
| 126 | 20A2 VL CDR2 | artificial | QDRKRPS |
| 127 | 20A2 VL CDR3 | artificial | QAWDSSTEV |
| 128 | 20A2 light chain | artificial | MAWALLLLTLLTQGTGSWASYELTQPPSVSVSPGQTASITCSGDKLGDKYACWYQQKPGQSPVL |
| | | | VIYQDRKRPSGIPERFSGSNSGNTATLTISGTQAMDEADYYCQAWDSSTEVFGGGT |
| | | | KLTVLGQPKAAPSVTLFPPSSEELQANKATLVCLISDFYPGAVTVAWKADSSPVKAGVETTTPS |
| | | | KQSNNKYAASSYLSLTPEQWKSHRSYSCQVTHEGSTVEKTVAPTECS |
| 129 | 2OC1 VH CDR1 | artificial | SYDMS |
| 130 | 20C1 VH CDR2 | artificial | LISGGGSNTYYADSVKG |
| 131 | 20C1 VH CDR3 | artificial | PSGHYFYAMDV |
| 132 | 20C1 VL CDR1 | artificial | RASQGISNWLA |
| 133 | 20C1 VL CDR2 | artificial | AASSLQS |
| 134 | 22D4 VH CDR1 | artificial | DYSMS |
| 135 | 22D4 VH CDR2 | artificial | GINWNGGRTRYAD SVKG |
| 136 | 22D4 VH CDR3 | artificial | EFNNFESNWFDP |
| 137 | 22D4 VL CDR1 | artificial | SGDALPKKYAY |
| 138 | 22D4 VL CDR2 | artificial | EDSKRPS |
| 139 | 22D4 VL CDR3 | artificial | YSTDSSGNHRV |
| 140 | 20C1.006 VH CDR1 | artificial | SYDMS |
| 141 | 20C1.006 VH CDR2 | artificial | LISGGGSNTYYAESVKG |
| 142 | 20C1.006 VH CDR3 | artificial | PSGHYFYAMDV |
| 143 | 20C1.006 VL CDR1 | artificial | RASQGISNWLA |
| 144 | 20C1.006 VL CDR2 | artificial | AASSLQS |
| 145 | 20C1.006 VL CDR3 | artificial | QQAESFPHT |
| 146 | 20C1.009 VH CDR1 | artificial | SYDMS |
| 147 | 20C1.009 VH CDR2 | artificial | LISGGGSQTYYAESVKG |
| 148 | 20C1.009 VH CDR3 | artificial | PSGHYFYAMDV |
| 149 | 20C1.009 VL CDR1 | artificial | RASQGISNWLA |
| 150 | 20C1.009 VL CDR2 | artificial | AASSLQS |
| 151 | 20C1.009 VL CDR3 | artificial | QQAESFPHT |
| 152 | 20A2.003 VH CDR1 | artificial | NYGMH |
| 153 | 20A2.003 VH CDR2 | artificial | LIWYDASKKYYAESVKG |
| 154 | 20A2.003 VH CDR3 | artificial | DPSSLTGSTGYYGMDV |
| 155 | 20A2.003 VL CDR1 | artificial | SGDKLGDKYAS |
| 156 | 20A2.003 VL CDR2 | artificial | QDRKRPS |
| 157 | 20A2.003 VL CDR3 | artificial | QAFESSTEV |
| 158 | 22D4.006 VH CDR1 | artificial | DYSMS |
| 159 | 22D4.006 VH CDR2 | artificial | GINWNGGRTRYADAVKG |
| 160 | 22D4.006 VH CDR3 | artificial | EFNNFESNWFDP |
| 161 | 22D4.006 VL CDR1 | artificial | SGDALPKKYAY |
| 162 | 22D4.006 VL CDR2 | artificial | EDAKRPS |
| 163 | 22D4.006 VL CDR3 | artificial | YSTDASGNHRV |
| 164 | 22D4.017 VH CDR1 | artificial | DYSMS |
| 165 | 22D4.017 VH CDR2 | artificial | GINWNAGRTRYADAVKG |
| 166 | 22D4.017 VH CDR3 | artificial | EFNNFESNWFDP |
| 167 | 22D4.017 VL CDR1 | artificial | SGDALPKKYAY |
| 168 | 22D4.017 VL CDR2 | artificial | EDAKRPS |
| 169 | 22D4.017 VL CDR3 | artificial | YSTDASGNHRV |
| 170 | 20A2 VH | artificial | QVQLVESGGGVVQPGRSLRLSCAASGFTFSNYGMHWVRQAPGKGLEWVALIWYDGSKKYYADSV |
| | | | KGRFTISRDNSKNTLYLQMNSLRAEDTAAYYCARDPSSLTGSTGYYGMDVWGQGTTVTVSS |
| 171 | 20A2 VL | artificial | SYELTQPPSVSVSPGQTASITCSGDKLGDKYACWYQQKPGQSPVLVIYQDRKRPSGIPERFSGS |
| | | | NSGNTATLTISGTQAMDEADYYCQAWDSSTEVFGGGTKLTVL |
| 172 | 20C1 VH | artificial | EVQLLESGGGLVQPGGALRLSCAASGFTFSSYDMSWVRQAPGKGLEWVSLISGGGSNTYYADSV |
| | | | KGRFTISRDNSKNTLYLQMNSLRAEDTAVYFCASPSGHYFYAMDVWGQGTTVTVSS |
| 173 | 20C1 VL | artificial | DIQMTQSPSSVSASVGDRVTITCRASQGISNWLAWYQQKPGKAPKLLIFAASSLQSGVPSRFGG |
| | | | SGSGTDFTFTISSLQPEDFATYYCQQADSFPHTFGGGTKVEIK |
| 174 | 22D4 VH | artificial | EVQLVESGGSVVRPGGSLRLSCAASGFTVDDYSMSWVRQVPGKGLEWVSGINWNGGRTRYADSV |
| | | | KGRFTISRDSAKNSLYLQMNSLRAEDTALYYCAREFNNFESNWFDPWGQGTLVTVSS |
| 175 | 22D4 VL | artificial | SYELTQPPSVSVSPGQTARITCSGDALPKKYAYWYQQKSGQAPVLVISEDSKRPSGIPERFSGS |
| | | | SSGTMATLTISGAQVEDEADYYCYSTDSSGNHRVFGGGTKLTVL |
| 176 | 20C1.006 VH | artificial | EVQLLESGGGLVQPGGSLRLSCAASGFTFSSYDMSWVRQAPGKGLEWVSLISGGGSNTYYAESV |
| | | | KGRFTISRDNSKNTLYLQMNSLRAEDTAVYFCASPSGHYFYAMDVWGQGTTVTVSS |
| 177 | 20C1.006 VL | artificial | DIQMTQSPSSVSASVGDRVTITCRASQGISNWLAWYQQKPGKAPKLLIFAASSLQSGVPSRFSG |
| | | | SGSGTDFTLTISSLQPEDFATYYCQQAESFPHTFGGGTKVEIK |
| 178 | 20C1.009 VH | artificial | EVQLLESGGGLVQPGGSLRLSCAASGFTFSSYDMSWVRQAPGKGLEWVSLISGGGSQTYYAESV |
| | | | KGRFTISRDNSKNTLYLQMNSLRAEDTAVYFCASPSGHYFYAMDVWGQGTTVTVSS |
| 179 | 20C1.009 VL | artificial | DIQMTQSPSSVSASVGDRVTITCRASQGISNWLAWYQQKPGKAPKLLIFAASSLQSGVPSRFSG |
| | | | SGSGTDFTLTISSLQPEDFATYYCQQAESFPHTFGGGTKVEIK |
| 180 | 20A2.003 VH | artificial | QVQLVESGGGWQPGRSLRLSCAASGFTFSNYGMHWVRQAPGKGLEWVALIWYDASKKYYAESV |
| | | | KGRFTISRDNSKNTLYLQMNSLRAEDTAAYYCARDPSSLTGSTGYYGMDVWGQGTTVTVSS |
| 181 | 20A2.003 VL | artificial | SYELTQPPSVSVSPGQTASITCSGDKLGDKYASWYQQKPGQSPVLVIYQDRKRPSGIPERFSGS |
| | | | NSGNTATLTISGTQAMDEADYYCQAFESSTEVFGGGTKLTVL |
| 182 | 22D4.006 VH | artificial | EVQLVESGGSVVRPGGSLRLSCAASGFTVDDYSMSWVRQVPGKGLEWVSGINWNGGRTRYADAV |
| | | | KGRFTISRDSAKNSLYLQMNSLRAEDTALYYCAREFNNFESNWFDPWGQGTLVTVSS |
| 183 | 22D4.006 VL | artificial | SYELTQPPSVSVSPGQTARITCSGDALPKKYAYWYQQKPGQAPVLVISEDAKRPSGIPERFSGS |
| | | | SSGTMATLTISGAQVEDEADYYCYSTDASGNHRVFGGGTKLTVL |
| 184 | 22D4.017 VH | artificial | EVQLVESGGSVVRPGGSLRLSCAASGFTVDDYSMSWVRQVPGKGLEWVSGINWNAGRTRYADAV |
| | | | KGRFTISRDSAKNSLYLQMNSLRAEDTALYYCAREFNNFESNWFDPWGQGTLVTVSS |
| 185 | 22D4.017 VL | artificial | SYELTQPPSVSVSPGQTARITCSGDALPKKYAYWYQQKPGQAPVLVISEDAKRPSGIPERFSGS |
| | | | SSGTMATLTISGAQVEDEADYYCYSTDASGNHRVFGGGTKLTVL |
| 186 | 20A2 heavy chain | artificial | MDMRVPAQLLGLLLLWLRGARCQVQLVESGGGVVQPGRSLRLSCAASGFTFSNYGMHWVRQAPG |
| | | | KGLEWVALIWYDGSKKYYADSVKGRFTISRDNSKNTLYLQMNSLRAEDTAAYYCAR |
| | | | DPSSLTGSTGYYGMDVWGQGTTVTVSSASTKGPSVFPLAPSSKSTSGGTAALGCLVKDYFPEPV |
| | | | TVSWNSGALTSGVHTFPAVLQSSGLYSLSSVVTVPSSSLGTQTYICNVNHKPSNTK |
| | | | VDKKVEPKSCDKTHTCPPCPAPELLGGPSVFLFPPKPKDTLMISRTPEVTCVVVDVSHEDPEVK |
| | | | FNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPENNYKTT |
| | | | PPVLDSDGSFFLYSKLTVDKSRWQQGNVFSCSVMHEALHNHYTQKSLSLSPGK |
| 187 | 20A2 light chain | artificial | MAWALLLLTLLTQGTGSWASYELTQPPSVSVSPGQTASITCSGDKLGDKYACWYQQKPGQSPVL |
| | | | VIYQDRKRPSGIPERFSGSNSGNTATLTISGTQAMDEADYYCQAWDSSTEVFGGGT |
| | | | KLTVLGQPKAAPSVTLFPPSSEELQANKATLVCLISDFYPGAVTVAWKADSSPVKAGVETTTPS |
| | | | KQSNNKYAASSYLSLTPEQWKSHRSYSCQVTHEGSTVEKTVAPTECS |
| 188 | 20C1 heavy chain | artificial | MDMRVPAQLLGLLLLWLRGARCEVQLLESGGGLVQPGGALRLSCAASGFTFSSYDMSWVRQAPG |
| | | | KGLEWVSLISGGGSNTYYADSVKGRFTISRDNSKNTLYLQMNSLRAEDTAVYFCAS |
| | | | PSGHYFYAMDVWGQGTTVTVSSASTKGPSVFPLAPSSKSTSGGTAALGCLVKDYFPEPVTVSWN |
| | | | SGALTSGVHTFPAVLQSSGLYSLSSVVTVPSSSLGTQTYICNVNHKPSNTKVDKKV |
| | | | EPKSCDKTHTCPPCPAPELLGGPSVFLFPPKPKDTLMISRTPEVTCVVVDVSHEDPEVKFNWYV |
| | | | DGVEVHNAKTKPCEEQYGSTYRCVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKTTPPVLD |
| | | | SDGSFFLYSKLTVDKSRWQQGNVFSCSVMHEALHNHYTQKSLSLSPGK |
| 189 | 20C1 light chain | artificial | MDMRVPAQLLGLLLLWLRGARCDIQMTQSPSSVSASVGDRVTITCRASQGISNWLAWYQQKPGK |
| | | | APKLLIFAASSLQSGVPSRFGGSGSGTDFTFTISSLQPEDFATYYCQQADSFPHTF |
| | | | GGGTKVEIKRTVAAPSVFIFPPSDEQLKSGTASVVCLLNNFYPREAKVQWKVDNALQSGNSQES |
| | | | VTEQDSKDSTYSLSSTLTLSKADYEKHKVYACEVTHQGLSSPVTKSFNRGEC |
| 190 | 22D4 heavy chain | artificial | MDMRVPAQLLGLLLLWLRGARCEVQLVESGGSVVRPGGSLRLSCAASGFTVDDYSMSWVRQVPG |
| | | | KGLEWVSGINWNGGRTRYADSVKGRFTISRDSAKNSLYLQMNSLRAEDTALYYCAR |
| | | | EFNNFESNWFDPWGQGTLVTVSSASTKGPSVFPLAPSSKSTSGGTAALGCLVKDYFPEPVTVSW |
| | | | NSGALTSGVHTFPAVLQSSGLYSLSSVVTVPSSSLGTQTYICNVNHKPSNTKVDKK |
| | | | VEPKSCDKTHTCPPCPAPELLGGPSVFLFPPKPKDTLMISRTPEVTCVVVDVSHEDPEVKFNWY |
| | | | VDGVEVHNAKTKPCEEQYGSTYRCVSVLTVLHQDWLNGKEYKCKVSNKALPAPIEK |
| | | | TISKAKGQPREPQVYTLPPSREEMTKNQVSLTCLVKGFYPSDIAVEWESNGQPENNYKTTPPVL |
| | | | DSDGSFFLYSKLTVDKSRWQQGNVFSCSVMHEALHNHYTQKSLSLSPGK |
| 191 | 22D4 light chain | artificial | MAWALLLLTLLTQGTGSWASYELTQPPSVSVSPGQTARITCSGDALPKKYAYWYQQKSGQAPVL |
| | | | VISEDSKRPSGIPERFSGSSSGTMATLTISGAQVEDEADYYCYSTDSSGNHRVFGG |
| | | | GTKLTVLGQPKAAPSVTLFPPSSEELQANKATLVCLISDFYPGAVTVAWKADSSPVKAGVETTT |
| | | | PSKQSNNKYAASSYLSLTPEQWKSHRSYSCQVTHEGSTVEKTVAPTECS |
| 192 | 20C1. 006 | artificial | MDMRVPAQLLGLLLLWLRGARCEVQLLESGGGLVQPGGSLRLSCAASGFTFSSYDMSWVRQAPG |
| | heavy chain | | KGLEWVSLISGGGSNTYYAESVKGRFTISRDNSKNTLYLQMNSLRAEDTAVYFCAS |
| | | | PSGHYFYAMDVWGQGTTVTVSSASTKGPSVFPLAPSSKSTSGGTAALGCLVKDYFPEPVTVSWN |
| | | | SGALTSGVHTFPAVLQSSGLYSLSSVVTVPSSSLGTQTYICNVNHKPSNTKVDKKV |
| | | | EPKSCDKTHTCPPCPAPELLGGPSVFLFPPKPKDTLMISRTPEVTCVVVDVSHEDPEVKFNWYV |
| | | | DGVEVHNAKTKPCEEQYGSTYRCVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKTTPPVLD |
| | | | SDGSFFLYSKLTVDKSRWQQGNVFSCSVMHEALHNHYTQKSLSLSPGK |
| 193 | 20C1.006 | artificial | MDMRVPAQLLGLLLLWLRGARCDIQMTQSPSSVSASVGDRVTITCRASQGISNWLAWYQQKPGK |
| | light chain | | APKLLIFAASSLQSGVPSRFSGSGSGTDFTLTISSLQPEDFATYYCQQAESFPHTF |
| | | | GGGTKVEIKRTVAAPSVFIFPPSDEQLKSGTASVVCLLNNFYPREAKVQWKVDNALQSGNSQES |
| | | | VTEQDSKDSTYSLSSTLTLSKADYEKHKVYACEVTHQGLSSPVTKSFNRGEC |
| 194 | 20C1.009 | artificial | MDMRVPAQLLGLLLLWLRGARCEVQLLESGGGLVQPGGSLRLSCAASGFTFSSYDMSWVRQAPG |
| | heavy chain | | KGLEWVSLISGGGSQTYYAESVKGRFTISRDNSKNTLYLQMNSLRAEDTAVYFCAS |
| | | | PSGHYFYAMDVWGQGTTVTVSSASTKGPSVFPLAPSSKSTSGGTAALGCLVKDYFPEPVTVSWN |
| | | | SGALTSGVHTFPAVLQSSGLYSLSSVVTVPSSSLGTQTYICNVNHKPSNTKVDKKV |
| | | | EPKSCDKTHTCPPCPAPELLGGPSVFLFPPKPKDTLMISRTPEVTCVVVDVSHEDPEVKFNWYV |
| | | | DGVEVHNAKTKPCEEQYGSTYRCVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKTTPPVLD |
| | | | SDGSFFLYSKLTVDKSRWQQGNVFSCSVMHEALHNHYTQKSLSLSPGK |
| 195 | 20C1.009 | artificial | MDMRVPAQLLGLLLLWLRGARCDIQMTQSPSSVSASVGDRVTITCRASQGISNWLAWYQQKPGK |
| | light chain | | APKLLIFAASSLQSGVPSRFSGSGSGTDFTLTISSLQPEDFATYYCQQAESFPHTF |
| | | | GGGTKVEIKRTVAAPSVFIFPPSDEQLKSGTASVVCLLNNFYPREAKVQWKVDNALQSGNSQES |
| | | | VTEQDSKDSTYSLSSTLTLSKADYEKHKVYACEVTHQGLSSPVTKSFNRGEC |
| 196 | 20A2.003 | artificial | MDMRVPAQLLGLLLLWLRGARCQVQLVESGGGVVQPGRSLRLSCAASGFTFSNYGMHWVRQAPG |
| | heavy chain | | KGLEWVALIWYDASKKYYAESVKGRFTISRDNSKNTLYLQMNSLRAEDTAAYYCAR |
| | | | DPSSLTGSTGYYGMDVWGQGTTVTVSSASTKGPSVFPLAPSSKSTSGGTAALGCLVKDYFPEPV |
| | | | TVSWNSGALTSGVHTFPAVLQSSGLYSLSSVVTVPSSSLGTQTYICNVNHKPSNTK |
| | | | VDKKVEPKSCDKTHTCPPCPAPELLGGPSVFLFPPKPKDTLMISRTPEVTCVVVDVSHEDPEVK |
| | | | FNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPENNYKTT |
| | | | PPVLDSDGSFFLYSKLTVDKSRWQQGNVFSCSVMHEALHNHYTQKSLSLSPGK |
| 197 | 20A2.003 | artificial | MAWALLLLTLLTQGTGSWASYELTQPPSVSVSPGQTASITCSGDKLGDKYASWYQQKPGQSPVL |
| | light chain | | VIYQDRKRPSGIPERFSGSNSGNTATLTISGTQAMDEADYYCQAFESSTEVFGGGT |
| | | | KLTVLGQPKAAPSVTLFPPSSEELQANKATLVCLISDFYPGAVTVAWKADSSPVKAGVETTTPS |
| | | | KQSNNKYAASSYLSLTPEQWKSHRSYSCQVTHEGSTVEKTVAPTECS |
| 198 | 22D4.006 | artificial | MDMRVPAQLLGLLLLWLRGARCEVQLVESGGSVVRPGGSLRLSCAASGFTVDDYSMSWVRQVPG |
| | heavy chain | | KGLEWVSGINWNGGRTRYADAVKGRFTISRDSAKNSLYLQMNSLRAEDTALYYCAR |
| | | | EFNNFESNWFDPWGQGTLVTVSSASTKGPSVFPLAPSSKSTSGGTAALGCLVKDYFPEPVTVSW |
| | | | NSGALTSGVHTFPAVLQSSGLYSLSSVVTVPSSSLGTQTYICNVNHKPSNTKVDKK |
| | | | VEPKSCDKTHTCPPCPAPELLGGPSVFLFPPKPKDTLMISRTPEVTCVVVDVSHEDPEVKFNWY |
| | | | VDGVEVHNAKTKPCEEQYGSTYRCVSVLTVLHQDWLNGKEYKCKVSNKALPAPIEK |
| | | | TISKAKGQPREPQVYTLPPSREEMTKNQVSLTCLVKGFYPSDIAVEWESNGQPENNYKTTPPVL |
| | | | DSDGSFFLYSKLTVDKSRWQQGNVFSCSVMHEALHNHYTQKSLSLSPGK |
| 199 | 22D4.006 | artificial | MAWALLLLTLLTQGTGSWASYELTQPPSVSVSPGQTARITCSGDALPKKYAYWYQQKPGQAPVL |
| | light chain | | VISEDAKRPSGIPERFSGSSSGTMATLTISGAQVEDEADYYCYSTDASGNHRVFGG |
| | | | GTKLTVLGQPKAAPSVTLFPPSSEELQANKATLVCLISDFYPGAVTVAWKADSSPVKAGVETTT |
| | | | PSKQSNNKYAASSYLSLTPEQWKSHRSYSCQVTHEGSTVEKTVAPTECS |
| 200 | 22D4.017 | artificial | MDMRVPAQLLGLLLLWLRGARCEVQLVESGGSVVRPGGSLRLSCAASGFTVDDYSMSWVRQVPG |
| | heavy chain | | KGLEWVSGINWNAGRTRYADAVKGRFTISRDSAKNSLYLQMNSLRAEDTALYYCAR |
| | | | EFNNFESNWFDPWGQGTLVTVSSASTKGPSVFPLAPSSKSTSGGTAALGCLVKDYFPEPVTVSW |
| | | | NSGALTSGVHTFPAVLQSSGLYSLSSVVTVPSSSLGTQTYICNVNHKPSNTKVDKK |
| | | | VEPKSCDKTHTCPPCPAPELLGGPSVFLFPPKPKDTLMISRTPEVTCVVVDVSHEDPEVKFNWY |
| | | | VDGVEVHNAKTKPCEEQYGSTYRCVSVLTVLHQDWLNGKEYKCKVSNKALPAPIEK |
| | | | TISKAKGQPREPQVYTLPPSREEMTKNQVSLTCLVKGFYPSDIAVEWESNGQPENNYKTTPPVL |
| | | | DSDGSFFLYSKLTVDKSRWQQGNVFSCSVMHEALHNHYTQKSLSLSPGK |
| 201 | 22D4.017 | artificial | MAWALLLLTLLTQGTGSWASYELTQPPSVSVSPGQTARITCSGDALPKKYAYWYQQKPGQAPVL |
| | light chain | | VISEDAKRPSGIPERFSGSSSGTMATLTISGAQVEDEADYYCYSTDASGNHRVFGG |
| | | | GTKLTVLGQPKAAPSVTLFPPSSEELQANKATLVCLISDFYPGAVTVAWKADSSPVKAGVETTT |
| | | | PSKQSNNKYAASSYLSLTPEQWKSHRSYSCQVTHEGSTVEKTVAPTECS |
| 202 | linker 1 | artificial | GGGG |
| 203 | linker 2 | artificial | GGGGS |
| 204 | linker 3 | artificial | GGGGQ |
| 205 | linker 4 | artificial | SGGGGS |
| 206 | linker 5 | artificial | PGGGGS |
| 207 | linker 6 | artificial | PGGDGS |
| 208 | linker 7 | artificial | GGGGSGGGS |
| 209 | linker 8 = (G₄S)₂ | artificial | GGGGSGGGGS |
| | linker | | |
| 210 | linker 9 = (G₄S)₃ | artificial | GGGGSGGGGSGGGGS |
| | linker | | |
| 211 | (G₄S)₄ linker | artificial | GGGGSGGGGSGGGGSGGGGS |
| 212 | (G₄S)₅ linker | artificial | GGGGSGGGGSGGGGSGGGGSGGGGS |
| 213 | (G₄S) linker | artificial | GGGGSGGGGSGGGGSGGGGSGGGGSGGGGS |
| 214 | (G₄S)₇ linker | artificial | GGGGSGGGGSGGGGSGGGGSGGGGSGGGGSGGGGS |
| 215 | (G₄S)₈ linker | artificial | GGGGSGGGGSGGGGSGGGGSGGGGSGGGGSGGGGSGGGGS |
| 216 | Ab156 | artificial | RDWDFDVFGGGTPVGG |
| 217 | linear FcRn BP | artificial | QRFVTGHFGGLXPANG |
| 218 | linear FcRn BP-Y | artificial | QRFVTGHFGGLYPANG |
| 219 | linear FcRn BP-H | artificial | QRFVTGHFGGLHPANG |
| 220 | core FcRn BP-H | artificial | TGHFGGLHP |
| 221 | cyclic FcRn BP-H | artificial | QRFCTGHFGGLHPCNG |
| 222 | HALB | human | DAHKSEVAHRFKDLGEENFKALVLIAFAQYLQQCPFEDHVKLVNEVTEFAKTCVADESAENCDK |
| | | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALEVDETYVPKEFNAETFTFHAD |
| | | | ICTLSEKERQIKKQTALVELVKHKPKATKEQLKAVMDDFAAFVEKCCKADDKETCFAEEGKKLV |
| | | | AASQAALGL |
| 223 | HALB | artificial | DAHKSEVAHRFKDLGEENFKALVLIAFAQYLQQCPFEDHVKLVNEVTEFAKTCVADESAENCDK |
| | variant 1 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALEVDETYVPKEFNAGTFTFHAD |
| | | | ICTLSEKERQIKKQTALVELVKHKPKATKEQLKAAMDDFAAFVEKCCKADDKETCFAEEGKKLV |
| | | | AASQAALGL |
| 224 | HALB | artificial | DAHKSEVAHRFKDLGEENFKALVLIAFAQYLQQCPFEDHVKLVNEVTEFAKTCVADESAENCDK |
| | variant 2 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALEVDETYVPKEFNAETFTFHAD |
| | | | ICTLSEKERQIKKQTALVELVKHKPKATKEQLKAVMDDFAAFVEKCCKADDKETCFAEEGPKLV |
| | | | AASQAALGL |
| 225 | HALB | artificial | DAHKSEVAHRFKDLGEENFKALVLIAFAQYLQQCPFEDHVKLVNEVTEFAKTCVADESAENCDK |
| | variant 3 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALDVDETYVPKEFNAETFTFHAD |
| | | | ICTLSEKERQIKKQTALVELVKHKPKATKEQLKAVMDDFAAFVEKCCKADDKETCFAEEGPHLV |
| | | | AASKAALGL |
| 226 | HALB | artificial | DAHKSEVAHRFKDLGEENFKALVLIAFAQYLQQCPFEDHVKLVNEVTEFAKTCVADESAENCDK |
| | variant 4 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALGVDETYVPKEFNAETFTFHAD |
| | | | ICTLSEKERQIKKQTALVELVKHKPKATKEQLKAVMDKFAAFVEKCCKADDKETCFAEEGPKLV |
| | | | AASQAALGL |
| 227 | HALB | artificial | DAHKSEVAHRFKDLGEENFKALVLIAFAQYLQQCPFEDHVKLVNEVTEFAKTCVADESAENCDK |
| | variant 5 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALDVDETYVPKEFNAETFTFHAD |
| | | | ICTLSEKERQIKKQTALVELVKHKPKATKEQLKAVMDKFAAFVEKCCKADDKETCFAEEGPKLV |
| | | | AASQAALGL |
| 228 | HALB | artificial | DAHKSEVAHRFKDLGEENFKALVLIAFAQYLQQCPFEDHVKLVNEVTEFAKTCVADESAENCDK |
| | variant 6 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALEVDETYVPKEFNAETFTFHAD |
| | | | ICTLSEKERQIKKQTALVELVKHKPKATKEQLKAVMDKFAAFVEKCCKADDKETCFAEEGPHLV |
| | | | AASQAALGL |
| 229 | HALB | artificial | DAHKSEVAHRFKDLGEENFKALVLIAFAQYLQQCPFEDHVKLVNEVTEFAKTCVADESAENCDK |
| | variant 7 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALEVDETYVPKEFNAETFTFHAD |
| | | | ICTLSEKERQIKKQTALVELVKHKPKATKEQLKAVMDDFAAFVEKCCKADDKETCFAEEGPHLV |
| | | | AASKAALGL |
| 230 | HALB | artificial | DAHKSEVAHRFKDLGEENFKALVLIAFAQYLQQCPFEDHVKLVNEVTEFAKTCVADESAENCDK |
| | variant 8 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALEVDETYVPKEFNAETFTFHAD |
| | | | ICTLSEKERQIKKQTALVELVKHKPKATKEQLKAVMDKFAAFVEKCCKADDKETCFAEEGPKLV |
| | | | AASKAALGL |
| 231 | HALB | artificial | DAHKSEVAHRFKDLGEENFKALVLIAFAQYLQQCPFEDHVKLVNEVTEFAKTCVADESAENCDK |
| | variant 9 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALDVDETYVPKEFNAETFTFHAD |
| | | | ICTLSEKERQIKKQTALVELVKHKPKATKEQLKAVMDDFAAFVEKCCKADDKETCFAEEGPKLV |
| | | | AASKAALGL |
| 232 | HALB | artificial | DAHKSEVAHRFKDLGEENFKALVLIAFAQYLQQSPFEDHVKLVNEVTEFAKTCVADESAENCDK |
| | variant 10 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALEVDETYVPKEFNAETFTFHAD |
| | | | ICTLSEKERQIKKQTALVELVKHKPKATKEQLKAVMDDFAAFVEKCCKADDKETCFAEEGKKLV |
| | | | AASQAALGL |
| 233 | HALB | artificial | DAHKSEVAHRFKDLGEENFKALVLIAFAQYLQQSPFEDHVKLVNEVTEFAKTCVADESAENCDK |
| | variant 11 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALEVDETYVPKEFNAGTFTFHAD |
| | | | ICTLSEKERQIKKQTALVELVKHKPKATKEQLKAAMDDFAAFVEKCCKADDKETCFAEEGKKLV |
| | | | AASQAALGL |
| 234 | HALB | artificial | DAHKSEVAHRFKDLGEENFKALVLIAFAQYLQQSPFEDHVKLVNEVTEFAKTCVADESAENCDK |
| | variant 12 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALEVDETYVPKEFNAETFTFHAD |
| | | | ICTLSEKERQIKKQTALVELVKHKPKATKEQLKAVMDDFAAFVEKCCKADDKETCFAEEGPKLV |
| | | | AASQAALGL |
| 235 | HALB | artificial | DAHKSEVAHRFKDLGEENFKALVLIAFAQYLQQSPFEDHVKLVNEVTEFAKTCVADESAENCDK |
| | variant 13 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALDVDETYVPKEFNAETFTFHAD |
| | | | ICTLSEKERQIKKQTALVELVKHKPKATKEQLKAVMDDFAAFVEKCCKADDKETCFAEEGPHLV |
| | | | AASKAALGL |
| 236 | HALB | artificial | DAHKSEVAHRFKDLGEENFKALVLIAFAQYLQQSPFEDHVKLVNEVTEFAKTCVADESAENCDK |
| | variant 14 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALGVDETYVPKEFNAETFTFHAD |
| | | | ICTLSEKERQIKKQTALVELVKHKPKATKEQLKAVMDKFAAFVEKCCKADDKETCFAEEGPKLV |
| | | | AASQAALGL |
| 237 | HALB | artificial | DAHKSEVAHRFKDLGEENFKALVLIAFAQYLQQSPFEDHVKLVNEVTEFAKTCVADESAENCDK |
| | variant 15 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALDVDETYVPKEFNAETFTFHAD |
| | | | ICTLSEKERQIKKQTALVELVKHKPKATKEQLKAVMDKFAAFVEKCCKADDKETCFAEEGPKLV |
| | | | AASQAALGL |
| 238 | HALB | artificial | DAHKSEVAHRFKDLGEENFKALVLIAFAQYLQQSPFEDHVKLVNEVTEFAKTCVADESAENCDK |
| | variant 16 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALEVDETYVPKEFNAETFTFHAD |
| | | | ICTLSEKERQIKKQTALVELVKHKPKATKEQLKAVMDKFAAFVEKCCKADDKETCFAEEGPHLV |
| | | | AASQAALGL |
| 239 | HALB | artificial | DAHKSEVAHRFKDLGEENFKALVLIAFAQYLQQSPFEDHVKLVNEVTEFAKTCVADESAENCDK |
| | variant 17 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALEVDETYVPKEFNAETFTFHAD |
| | | | ICTLSEKERQIKKQTALVELVKHKPKATKEQLKAVMDDFAAFVEKCCKADDKETCFAEEGPHLV |
| | | | AASKAALGL |
| 240 | HALB | artificial | DAHKSEVAHRFKDLGEENFKALVLIAFAQYLQQSPFEDHVKLVNEVTEFAKTCVADESAENCDK |
| | variant 18 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALEVDETYVPKEFNAETFTFHAD |
| | | | ICTLSEKERQIKKQTALVELVKHKPKATKEQLKAVMDKFAAFVEKCCKADDKETCFAEEGPKLV |
| | | | AASKAALGL |
| 241 | HALB | artificial | DAHKSEVAHRFKDLGEENFKALVLIAFAQYLQQSPFEDHVKLVNEVTEFAKTCVADESAENCDK |
| | variant 19 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALDVDETYVPKEFNAETFTFHAD |
| | | | ICTLSEKERQIKKQTALVELVKHKPKATKEQLKAVMDDFAAFVEKCCKADDKETCFAEEGPKLV |
| | | | AASKAALGL |
| 242 | HALB | artificial | DAHKSEVAHRFKDLGEENFKALVLIAFAQYLQQAPFEDHVKLVNEVTEFAKTCVADESAENCDK |
| | variant 20 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALEVDETYVPKEFNAETFTFHAD |
| | | | ICTLSEKERQIKKQTALVELVKHKPKATKEQLKAVMDDFAAFVEKCCKADDKETCFAEEGKKLV |
| | | | AASQAALGL |
| 243 | HALB | artificial | DAHKSEVAHRFKDLGEENFKALVLIAFAQYLQQAPFEDHVKLVNEVTEFAKTCVADESAENCDK |
| | variant 21 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALEVDETYVPKEFNAGTFTFHAD |
| | | | ICTLSEKERQIKKQTALVELVKHKPKATKEQLKAAMDDFAAFVEKCCKADDKETCFAEEGKKLV |
| | | | AASQAALGL |
| 244 | HALB | artificial | DAHKSEVAHRFKDLGEENFKALVLIAFAQYLQQAPFEDHVKLVNEVTEFAKTCVADESAENCDK |
| | variant 22 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALEVDETYVPKEFNAETFTFHAD |
| | | | ICTLSEKERQIKKQTALVELVKHKPKATKEQLKAVMDDFAAFVEKCCKADDKETCFAEEGPKLV |
| | | | AASQAALGL |
| 245 | HALB | artificial | DAHKSEVAHRFKDLGEENFKALVLIAFAQYLQQAPFEDHVKLVNEVTEFAKTCVADESAENCDK |
| | variant 23 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALDVDETYVPKEFNAETFTFHAD |
| | | | ICTLSEKERQIKKQTALVELVKHKPKATKEQLKAVMDDFAAFVEKCCKADDKETCFAEEGPHLV |
| | | | AASKAALGL |
| 246 | HALB | artificial | DAHKSEVAHRFKDLGEENFKALVLIAFAQYLQQAPFEDHVKLVNEVTEFAKTCVADESAENCDK |
| | variant 24 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALGVDETYVPKEFNAETFTFHAD |
| | | | ICTLSEKERQIKKQTALVELVKHKPKATKEQLKAVMDKFAAFVEKCCKADDKETCFAEEGPKLV |
| | | | AASQAALGL |
| 247 | HALB | artificial | DAHKSEVAHRFKDLGEENFKALVLIAFAQYLQQAPFEDHVKLVNEVTEFAKTCVADESAENCDK |
| | variant 25 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALDVDETYVPKEFNAETFTFHAD |
| | | | ICTLSEKERQIKKQTALVELVKHKPKATKEQLKAVMDKFAAFVEKCCKADDKETCFAEEGPKLV |
| | | | AASQAALGL |
| 248 | HALB | artificial | DAHKSEVAHRFKDLGEENFKALVLIAFAQYLQQAPFEDHVKLVNEVTEFAKTCVADESAENCDK |
| | variant 26 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALEVDETYVPKEFNAETFTFHAD |
| | | | ICTLSEKERQIKKQTALVELVKHKPKATKEQLKAVMDKFAAFVEKCCKADDKETCFAEEGPHLV |
| | | | AASQAALGL |
| 249 | HALB | artificial | DAHKSEVAHRFKDLGEENFKALVLIAFAQYLQQAPFEDHVKLVNEVTEFAKTCVADESAENCDK |
| | variant 27 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALEVDETYVPKEFNAETFTFHAD |
| | | | ICTLSEKERQIKKQTALVELVKHKPKATKEQLKAVMDDFAAFVEKCCKADDKETCFAEEGPHLV |
| | | | AASKAALGL |
| 250 | HALB | artificial | DAHKSEVAHRFKDLGEENFKALVLIAFAQYLQQAPFEDHVKLVNEVTEFAKTCVADESAENCDK |
| | variant 28 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALEVDETYVPKEFNAETFTFHAD |
| | | | ICTLSEKERQIKKQTALVELVKHKPKATKEQLKAVMDKFAAFVEKCCKADDKETCFAEEGPKLV |
| | | | AASKAALGL |
| 251 | HALB | artificial | DAHKSEVAHRFKDLGEENFKALVLIAFAQYLQQAPFEDHVKLVNEVTEFAKTCVADESAENCDK |
| | variant 29 | | SLHTLFGDKLCTVATLRETYGEMADCCAKQEPERNECFLQHKDDNPNLPRLVRPEVDVMCTAFH |
| | | | DNEETFLKKYLYEIARRHPYFYAPELLFFAKRYKAAFTECCQAADKAACLLPKLDELRDEGKAS |
| | | | SAKQRLKCASLQKFGERAFKAWAVARLSQRFPKAEFAEVSKLVTDLTKVHTECCHGDLLECADD |
| | | | RADLAKYICENQDSISSKLKECCEKPLLEKSHCIAEVENDEMPADLPSLAADFVESKDVCKNYA |
| | | | EAKDVFLGMFLYEYARRHPDYSVVLLLRLAKTYETTLEKCCAAADPHECYAKVFDEFKPLVEEP |
| | | | QNLIKQNCELFEQLGEYKFQNALLVRYTKKVPQVSTPTLVEVSRNLGKVGSKCCKHPEAKRMPC |
| | | | AEDYLSVVLNQLCVLHEKTPVSDRVTKCCTESLVNRRPCFSALDVDETYVPKEFNAETFTFHAD |
| | | | ICTLSEKERQIKKQTALVELVKHKPKATKEQLKAVMDDFAAFVEKCCKADDKETCFAEEGPKLV |
| | | | AASKAALGL |
| 252 | Cross body 1 HC | artificial | ASTKGPSVFPLAPSSKSTSGGTAALGCLVKDYFPEPVTVSWNSGALTSGVHTFPAVLQSSGLYS |
| | | | LSSVVTVPSSSLGTQTYICNVNHKPSNTKVDKKVEPKSCDKTHTCPPCPAPELLGGPSVFLFPP |
| | | | KPKDTLMISRTPEVTCVVVDVSHEDPEVKFNWYVDGVEVHNAKTKPCEEQYGSTYRCVSVLTVL |
| | | | HQDWLNGKEYKCKVSNKALPAPIEKTISKAKGQPREPQVYTLPPSREEMTKNQVSLTCLVKGFY |
| | | | PSDIAVEWESNGQPENNYDTTPPVLDSDGSFFLYSDLTVDKSRWQQGNVFSCSVMHEALHNHYT |
| | | | QKSLSLSPGK |
| 253 | Cross body 1 LC | artificial | GQPKAAPSVTLFPPSSEELQANKATLVCLISDFYPGAVTVAWKADSSPVKAGVETTTPSKQSNN |
| | | | KYAASSYLSLTPEQWKSHRSYSCQVTHEGSTVEKTVAPTECSDKTHTCPPCPAPELLGGPSVFL |
| | | | FPPKPKDTLMISRTPEVTCVVVDVSHEDPEVKFNWYVDGVEVHNAKTKPCEEQYGSTYRCVSVL |
| | | | TVLHQDWLNGKEYKCKVSNKALPAPIEKTISKAKGQPREPQVYTLPPSRKEMTKNQVSLTCLVK |
| | | | GFYPSDIAVEWESNGQPENNYKTTPPVLKSDGSFFLYSKLTVDKSRWQQGNVFSCSVMHEALHN |
| | | | HYTQKSLSLSPGK |
| 254 | Cross body 2 HC | artificial | ASTKGPSVFPLAPCSRSTSESTAALGCLVKDYFPEPVTVSWNSGALTSGVHTFPAVLQSSGLYS |
| | | | LSSVVTVPSSNFGTQTYTCNVDHKPSNTKVDKTVEPKSSDKTHTCPPCPAPEAAGGPSVFLFPP |
| | | | KPKDTLMISRTPEVTCVWDVSHEDPEVKFNWYVDGVEVHNAKTKPREEQYNSTYRWSVLTVL |
| | | | HQDWLNGKEYKCKVSNKALPAPIEKTISKAKGQPREPQVYTLPPSREEMTKNQVSLTCLVKGFY |
| | | | PSDIAVEWESNGQPENNYDTTPPVLDSDGSFFLYSDLTVDKSRWQQGNVFSCSVMHEALHNHYT |
| | | | QKSLSLSPGK |
| 255 | Cross body 2 LC | artificial | GQPKAAPSVTLFPPSSEELQANKATLVCLISDFYPGAVTVAWKADSSPVKAGVETTTPSKQSNN |
| | | | KYAASSYLSLTPEQWKSHRSYSCQVTHEGSTVEKTVAPTECSEPKSSDKTHTCPPCPAPEAAGG |
| | | | PSVFLFPPKPKDTLMISRTPEVTCVVVDVSHEDPEVKFNWYVDGVEVHNAKTKPREEQYNSTYR |
| | | | VVSVLTVLHQDWLNGKEYKCKVSNKALPAPIEKTISKAKGQPREPQVYTLPPSRKEMTKNQVSL |
| | | | TCLVKGFYPSDIAVEWESNGQPENNYKTTPPVLKSDGSFFLYSKLTVDKSRWQQGNVFSCSVMH |
| | | | EALHNHYTQKSLSLSPGK |
| 256 | Hetero-Fc | artificial | DKTHTCPPCPAPELLGGPSVFLFPPKPKDTLMISRTPEVTCVVVDVSHEDPEVKFNWYVDGVEV |
| | binder Fc | | HNAKTKPCEEQYGSTYRCVSVLTVLHQDWLNGKEYKCKVSNKALPAPIEKTISKAKGQPREPQV |
| | | | YTLPPSRKEMTKNQVSLTCLVKGFYPSDIAVEWESNGQPENNYKTTPPVLKSDGSFFLYSKLTV |
| | | | DKSRWQQGNVFSCSVMHEALHNHYTQKSLSLSPGK |
| 257 | Hetero-Fc | artificial | DKTHTCPPCPAPELLGGPSVFLFPPKPKDTLMISRTPEVTCVVVDVSHEDPEVKFNWYVDGVEV |
| | partner Fc | | HNAKTKPCEEQYGSTYRCVSVLTVLHQDWLNGKEYKCKVSNKALPAPIEKTISKAKGQPREPQV |
| | | | YTLPPSREEMTKNQVSLTCLVKGFYPSDIAVEWESNGQPENNYDTTPPVLDSDGSFFLYSDLTV |
| | | | DKSRWQQGNVFSCSVMHEALHNHYTQKSLSLSPGK |
| 258 | Maxi-body 1 | artificial | EPKSSDKTHTCPPCPAPELLGGPSVFLFPPKPKDTLMISRTPEVTCVVVDVSHEDPEVKFNWYV |
| | target Fc | | DGVEVHNAKTKPCEEQYGSTYRCVSVLTVLHQDWLNGKEYKCKVSNKALPAPIEKTISKAKGQP |
| | | | REPQVYTLPPSRKEMTKNQVSLTCLVKGFYPSDIAVEWESNGQPENNYKTTPPVLKSDGSFFLY |
| | | | SKLTVDKSRWQQGNVFSCSVMHEALHNHYTQKSLSLSPGK |
| 259 | Maxi-body 1 | artificial | EPKSSDKTHTCPPCPAPELLGGPSVFLFPPKPKDTLMISRTPEVTCVVVDVSHEDPEVKFNWYV |
| | CD3 Fc | | DGVEVHNAKTKPCEEQYGSTYRCVSVLTVLHQDWLNGKEYKCKVSNKALPAPIEKTISKAKGQP |
| | | | REPQVYTLPPSREEMTKNQVSLTCLVKGFYPSDIAVEWESNGQPENNYDTTPPVLDSDGSFFLY |
| | | | SDLTVDKSRWQQGNVFSCSVMHEALHNHYTQKSLSLSPGK |
| 260 | Maxi-body 2 | artificial | EPKSSDKTHTCPPCPAPEAAGGPSVFLFPPKPKDTLMISRTPEVTCVVVDVSHEDPEVKFNWYV |
| | target Fc | | DGVEVHNAKTKPREEQYNSTYRVVSVLTVLHQDWLNGKEYKCKVSNKALPAPIEKTISKAKGQP |
| | | | REPQVYTLPPSRKEMTKNQVSLTCLVKGFYPSDIAVEWESNGQPENNYKTTPPVLKSDGSFFLY |
| | | | SKLTVDKSRWQQGNVFSCSVMHEALHNHYTQKSLSLSPGK |
| 261 | Maxi-body 2 | artificial | EPKSSDKTHTCPPCPAPEAAGGPSVFLFPPKPKDTLMISRTPEVTCVVVDVSHEDPEVKFNWYV |
| | CD3 Fc | | DGVEVHNAKTKPREEQYNSTYRVVSVLTVLHQDWLNGKEYKCKVSNKALPAPIEKTISKAKGQP |
| | | | REPQVYTLPPSREEMTKNQVSLTCLVKGFYPSDIAVEWESNGQPENNYDTTPPVLDSDGSFFLY |
| | | | SDLTVDKSRWQQGNVFSCSVMHEALHNHYTQKSLSLSPGK |
| 262 | Mono Fc | artificial | APELLGGPSVFLFPPKPKDTLMISRTPEVTCVVVDVSHEDPEVKFNWYVDGVEVHNAKTKPREE |
| | | | QYNSTYRVVSVLTVLHQDWLNGKEYKCKVSNKALPAPIEKTISKAKGQPREPQVTTLPPSREEM |
| | | | TKNQVSLTCLVKGFYPSDIAVEWESNGQPENNYDTTPPVLDSDGSFFLYSDLTVDKSRWQQGNV |
| | | | FSCSVMHEALHNHYTQKSLSLSPGK |
| 263 | Fc monomer-1 | artificial | DKTHTCPPCPAPELLGGPSVFLFPPKPKDTLMISRTPEVTCVVVDVSHEDPEVKFNWYVDGVEV |
| | +c/−g | | HNAKTKPCEEQYGSTYRCVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPVLDSDGSFFLYSKLTV |
| | | | DKSRWQQGNVFSCSVMHEALHNHYTQKSLSLSPGK |
| 264 | Fc monomer-2 | artificial | DKTHTCPPCPAPELLGGPSVFLFPPKPKDTLMISRTPEVTCVVVDVSHEDPEVKFNWYVDGVEV |
| | +c/−g/delGK | | HNAKTKPCEEQYGSTYRCVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPVLDSDGSFFLYSKLTV |
| | | | DKSRWQQGNVFSCSVMHEALHNHYTQKSLSLSP |
| 265 | Fc monomer-3 | artificial | DKTHTCPPCPAPELLGGPSVFLFPPKPKDTLMISRTPEVTCVVVDVSHEDPEVKFNWYVDGVEV |
| | −c/+g | | HNAKTKPREEQYNSTYRVVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPVLDSDGSFFLYSKLTV |
| | | | DKSRWQQGNVFSCSVMHEALHNHYTQKSLSLSPGK |
| 266 | Fc monomer-4 | artificial | DKTHTCPPCPAPELLGGPSVFLFPPKPKDTLMISRTPEVTCVVVDVSHEDPEVKFNWYVDGVEV |
| | -d+d delGK | | HNAKTKPREEQYNSTYRVVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPVLDSDGSFFLYSKLTV |
| | | | DKSRWQQGNVFSCSVMHEALHNHYTQKSLSLSP |
| 267 | Fc monomer-5 | artificial | DKTHTCPPCPAPELLGGPSVFLFPPKPKDTLMISRTPEVTCVVVDVSHEDPEVKFNWYVDGVEV |
| | −c/−g | | HNAKTKPREEQYGSTYRVVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPVLDSDGSFFLYSKLTV |
| | | | DKSRWQQGNVFSCSVMHEALHNHYTQKSLSLSPGK |
| 268 | Fc monomer-6 | artificial | DKTHTCPPCPAPELLGGPSVFLFPPKPKDTLMISRTPEVTCVVVDVSHEDPEVKFNWYVDGVEV |
| | −c/−g/ delGK | | HNAKTKPREEQYGSTYRVVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPVLDSDGSFFLYSKLTV |
| | | | DKSRWQQGNVFSCSVMHEALHNHYTQKSLSLSP |
| 269 | Fc monomer-7 | artificial | DKTHTCPPCPAPELLGGPSVFLFPPKPKDTLMISRTPEVTCVVVDVSHEDPEVKFNWYVDGVEV |
| | +c/+g | | HNAKTKPCEEQYNSTYRCVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPVLDSDGSFFLYSKLTV |
| | | | DKSRWQQGNVFSCSVMHEALHNHYTQKSLSLSPGK |
| 270 | Fc monomer-8 | artificial | DKTHTCPPCPAPELLGGPSVFLFPPKPKDTLMISRTPEVTCVVVDVSHEDPEVKFNWYVDGVEV |
| | delGK | | HNAKTKPCEEQYNSTYRCVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPVLDSDGSFFLYSKLTV |
| | | | DKSRWQQGNVFSCSVMHEALHNHYTQKSLSLSP |
| 271 | scFc-l | artificial | DKTHTCPPCPAPELLGGPSVFLFPPKPKDTLMISRTPEVTCVVVDVSHEDPEVKFNWYVDGVEV |
| | | | HNAKTKPCEEQYGSTYRCVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPVLDSDGSFFLYSKLTV |
| | | | DKSRWQQGNVFSCSVMHEALHNHYTQKSLSLSPGKGGGGSGGGGSGGGGSGGGGSGGGGSGGGG |
| | | | SDKTHTCPPCPAPELLGGPSVFLFPPKPKDTLMISRTPEVTCVVVDVSHEDPEVKFNWYVDGVE |
| | | | VHNAKTKPCEEQYGSTYRCVSVLTVLHQDWLNGKEYKCKVSNKALPAPIEKTISKAKGQPREPQ |
| | | | VYTLPPSREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGK |
| 272 | scFc-2 | artificial | DKTHTCPPCPAPELLGGPSVFLFPPKPKDTLMISRTPEVTCVVVDVSHEDPEVKFNWYVDGVEV |
| | | | HNAKTKPCEEQYGSTYRCVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPVLDSDGSFFLYSKLTV |
| | | | DKSRWQQGNVFSCSVMHEALHNHYTQKSLSLSPGGGGSGGGGSGGGGSGGGGSGGGGSGGGGSD |
| | | | KTHTCPPCPAPELLGGPSVFLFPPKPKDTLMISRTPEVTCVVVDVSHEDPEVKFNWYVDGVEVH |
| | | | NAKTKPCEEQYGSTYRCVSVLTVLHQDWLNGKEYKCKVSNKALPAPIEKTISKAKGQPREPQVY |
| | | | TLPPSREEMTKNQVSLTCLVKGFYPSDIAVEWESNGQPENNYKTTPPVLDSDGSFFLYSKLTVD |
| | | | KSRWQQGNVFSCSVMHEALHNHYTQKSLSLSP |
| 273 | scFc-3 | artificial | DKTHTCPPCPAPELLGGPSVFLFPPKPKDTLMISRTPEVTCVVVDVSHEDPEVKFNWYVDGVEV |
| | | | HNAKTKPREEQYNSTYRVVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPVLDSDGSFFLYSKLTV |
| | | | DKSRWQQGNVFSCSVMHEALHNHYTQKSLSLSPGKGGGGSGGGGSGGGGSGGGGSGGGGSGGGG |
| | | | SDKTHTCPPCPAPELLGGPSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALPAPIEKTISKAKGQPREPQ |
| | | | VYTLPPSREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGK |
| 274 | scFc-4 | artificial | DKTHTCPPCPAPELLGGPSVFLFPPKPKDTLMISRTPEVTCVVVDVSHEDPEVKFNWYVDGVEV |
| | | | HNAKTKPREEQYNSTYRVVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPVLDSDGSFFLYSKLTV |
| | | | DKSRWQQGNVFSCSVMHEALHNHYTQKSLSLSPGGGGSGGGGSGGGGSGGGGSGGGGSGGGGSD |
| | | | KTHTCPPCPAPELLGGPSVFLFPPKPKDTLMISRTPEVTCVVVDVSHEDPEVKFNWYVDGVEVH |
| | | | NAKTKPREEQYNSTYRVVSVLTVLHQDWLNGKEYKCKVSNKALPAPIEKTISKAKGQPREPQVY |
| | | | TLPPSREEMTKNQVSLTCLVKGFYPSDIAVEWESNGQPENNYKTTPPVLDSDGSFFLYSKLTVD |
| | | | KSRWQQGNVFSCSVMHEALHNHYTQKSLSLSP |
| 275 | scFc-5 | artificial | DKTHTCPPCPAPELLGGPSVFLFPPKPKDTLMISRTPEVTCVVVDVSHEDPEVKFNWYVDGVEV |
| | | | HNAKTKPREEQYGSTYRVVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPVLDSDGSFFLYSKLTV |
| | | | DKSRWQQGNVFSCSVMHEALHNHYTQKSLSLSPGKGGGGSGGGGSGGGGSGGGGSGGGGSGGGG |
| | | | SDKTHTCPPCPAPELLGGPSVFLFPPKPKDTLMISRTPEVTCVVVDVSHEDPEVKFNWYVDGVE |
| | | | VHNAKTKPREEQYGSTYRVVSVLTVLHQDWLNGKEYKCKVSNKALPAPIEKTISKAKGQPREPQ |
| | | | VYTLPPSREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGK |
| 276 | scFc-6 | artificial | DKTHTCPPCPAPELLGGPSVFLFPPKPKDTLMISRTPEVTCVVVDVSHEDPEVKFNWYVDGVEV |
| | | | HNAKTKPREEQYGSTYRVVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPVLDSDGSFFLYSKLTV |
| | | | DKSRWQQGNVFSCSVMHEALHNHYTQKSLSLSPGGGGSGGGGSGGGGSGGGGSGGGGSGGGGSD |
| | | | KTHTCPPCPAPELLGGPSVFLFPPKPKDTLMISRTPEVTCVVVDVSHEDPEVKFNWYVDGVEVH |
| | | | NAKTKPREEQYGSTYRVVSVLTVLHQDWLNGKEYKCKVSNKALPAPIEKTISKAKGQPREPQVY |
| | | | TLPPSREEMTKNQVSLTCLVKGFYPSDIAVEWESNGQPENNYKTTPPVLDSDGSFFLYSKLTVD |
| | | | KSRWQQGNVFSCSVMHEALHNHYTQKSLSLSP |
| 277 | scFc-7 | artificial | DKTHTCPPCPAPELLGGPSVFLFPPKPKDTLMISRTPEVTCVVVDVSHEDPEVKFNWYVDGVEV |
| | | | HNAKTKPCEEQYNSTYRCVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPVLDSDGSFFLYSKLTV |
| | | | DKSRWQQGNVFSCSVMHEALHNHYTQKSLSLSPGKGGGGSGGGGSGGGGSGGGGSGGGGSGGGG |
| | | | SDKTHTCPPCPAPELLGGPSVFLFPPKPKDTLMISRTPEVTCVVVDVSHEDPEVKFNWYVDGVE |
| | | | VHNAKTKPCEEQYNSTYRCVSVLTVLHQDWLNGKEYKCKVSNKALPAPIEKTISKAKGQPREPQ |
| | | | VYTLPPSREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGK |
| 278 | scFc-8 | artificial | DKTHTCPPCPAPELLGGPSVFLFPPKPKDTLMISRTPEVTCVVVDVSHEDPEVKFNWYVDGVEV |
| | | | HNAKTKPCEEQYNSTYRCVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPVLDSDGSFFLYSKLTV |
| | | | DKSRWQQGNVFSCSVMHEALHNHYTQKSLSLSPGGGGSGGGGSGGGGSGGGGSGGGGSGGGGSD |
| | | | KTHTCPPCPAPELLGGPSVFLFPPKPKDTLMISRTPEVTCVVVDVSHEDPEVKFNWYVDGVEVH |
| | | | NAKTKPCEEQYNSTYRCVSVLTVLHQDWLNGKEYKCKVSNKALPAPIEKTISKAKGQPREPQVY |
| | | | TLPPSREEMTKNQVSLTCLVKGFYPSDIAVEWESNGQPENNYKTTPPVLDSDGSFFLYSKLTVD |
| | | | KSRWQQGNVFSCSVMHEALHNHYTQKSLSLSP |
| 279 | 5x his-tag | artificial | HHHHH |
| 280 | 6x his-tag | artificial | HHHHHH |
| 281 | CL-1 x I2C | bispecific | QVQLVQSGAEVKKPGASVKVSCKASGYTFTGYYMHWVRQAPGQCLEWMGWINPNSGGTKYAQKF |
| | | molecule | QGRVTMTRDTSISTAYMELSRLRSDDTAVYYCARDRITVAGTYYYYGMDVWGQGTTVTVSSGGG |
| | | | GSGGGGSGGGGSDIQMTQSPSSVSASVGDRVTITCRASQGVNNWLAWYQQKPGKAPKLLIYTAS |
| | | | SLQSGVPSRFSGSGSGTDFTLTIRSLQPEDFATYYCQQANSFPITFGCGTRLEIKSGGGGSEVQ |
| | | | LVESGGGLVQPGGSLKLSCAASGFTFNKYAMNWVRQAPGKGLEWVARIRSKYNNYATYYADSVK |
| | | | DRFTISRDDSKNTAYLQMNNLKTEDTAVYYCVRHGNFGNSYISYWAYWGQGTLVTVSSGGGGSG |
| | | | GGGSGGGGSQTVVTQEPSLTVSPGGTVTLTCGSSTGAVTSGNYPNWVQQKPGQAPRGLIGGTKF |
| | | | LAPGTPARFSGSLLGGKAALTLSGVQPEDEAEYYCVLWYSNRWVFGGGTKLTVL |
| 282 | CL-2 x I2C | bispecific | QVQMVQSGAEVKKHGASVKVSCKASGYTFTGYYMHWVRQAPGQCLEWMGWINPNSGGTKYAQKF |
| | | molecule | QGRVTMTRDTSISTAYMELSRLRSDDTAVYYCARDRITVAGTYYYYGMDVWGQGTTVTVSSGGG |
| | | | GSGGGGSGGGGSDIQMTQSPSSVSASVGDRVTITCRASQGVNNWLAWYQQKPGKAPKLLIYTAS |
| | | | SLQSGVPSRFSGSGSGTDFTLTIRSLQPEDFATYYCQQANSFPITFGCGTRLEIKSGGGGSEVQ |
| | | | LVESGGGLVQPGGSLKLSCAASGFTFNKYAMNWVRQAPGKGLEWVARIRSKYNNYATYYADSVK |
| | | | DRFTISRDDSKNTAYLQMNNLKTEDTAVYYCVRHGNFGNSYISYWAYWGQGTLVTVSSGGGGSG |
| | | | GGGSGGGGSQTVVTQEPSLTVSPGGTVTLTCGSSTGAVTSGNYPNWVQQKPGQAPRGLIGGTKF |
| | | | LAPGTPARFSGSLLGGKAALTLSGVQPEDEAEYYCVLWYSNRWVFGGGTKLTVL |
| 283 | CL-1 x I2C-6His | bispecific | QVQLVQSGAEVKKPGASVKVSCKASGYTFTGYYMHWVRQAPGQCLEWMGWINPNSGGTKYAQKF |
| | | molecule - | QGRVTMTRDTSISTAYMELSRLRSDDTAVYYCARDRITVAGTYYYYGMDVWGQGTTVTVSSGGG |
| | | his tag | GSGGGGSGGGGSDIQMTQSPSSVSASVGDRVTITCRASQGVNNWLAWYQQKPGKAPKLLIYTAS |
| | | | SLQSGVPSRFSGSGSGTDFTLTIRSLQPEDFATYYCQQANSFPITFGCGTRLEIKSGGGGSEVQ |
| | | | LVESGGGLVQPGGSLKLSCAASGFTFNKYAMNWVRQAPGKGLEWVARIRSKYNNYATYYADSVK |
| | | | DRFTISRDDSKNTAYLQMNNLKTEDTAVYYCVRHGNFGNSYISYWAYWGQGTLVTVSSGGGGSG |
| | | | GGGSGGGGSQTVVTQEPSLTVSPGGTVTLTCGSSTGAVTSGNYPNWVQQKPGQAPRGLIGGTKF |
| | | | LAPGTPARFSGSLLGGKAALTLSGVQPEDEAEYYCVLWYSNRWVFGGGTKLTVLHHHHHH |
| 284 | CL-2 x I2C-6His | bispecific | QVQMVQSGAEVKKHGASVKVSCKASGYTFTGYYMHWVRQAPGQCLEWMGWINPNSGGTKYAQKF |
| | | molecule - | QGRVTMTRDTSISTAYMELSRLRSDDTAVYYCARDRITVAGTYYYYGMDVWGQGTTVTVSSGGG |
| | | his tag | GSGGGGSGGGGSDIQMTQSPSSVSASVGDRVTITCRASQGVNNWLAWYQQKPGKAPKLLIYTAS |
| | | | SLQSGVPSRFSGSGSGTDFTLTIRSLQPEDFATYYCQQANSFPITFGCGTRLEIKSGGGGSEVQ |
| | | | LVESGGGLVQPGGSLKLSCAASGFTFNKYAMNWVRQAPGKGLEWVARIRSKYNNYATYYADSVK |
| | | | DRFTISRDDSKNTAYLQMNNLKTEDTAVYYCVRHGNFGNSYISYWAYWGQGTLVTVSSGGGGSG |
| | | | GGGSGGGGSQTVVTQEPSLTVSPGGTVTLTCGSSTGAVTSGNYPNWVQQKPGQAPRGLIGGTKF |
| | | | LAPGTPARFSGSLLGGKAALTLSGVQPEDEAEYYCVLWYSNRWVFGGGTKLTVLHHHHHH |
| 285 | AUl epitope | artificial | DTYRYI |
| 286 | AU5 epitope | artificial | TDFYLK |
| 287 | T-7 tag | artificial | MASMTGGQQMG |
| 288 | V-5 tag | artificial | GKPIPNPLLGLDST |
| 289 | B-tag | artificial | QYPALT |
| 290 | E2 epitope | artificial | SSTSSDFRDR |
| 291 | FLAG tag | artificial | DYKDDDK |
| 292 | Glu-Glu tag 1 | artificial | EYMPME |
| 293 | Glu-Glu tag 2 | artificial | EFMPME |
| 294 | Histidine | artificial | KDHLIHNVHKEFHAHAHNK |
| | affinity tag | | |
| 295 | HSV epitope | artificial | QPELAPED |
| 296 | KT3 epitope | artificial | KPPTPPPEPET |
| 297 | Myc epitope | artificial | CEQKLISEEDL |
| 298 | 7x his-tag | artificial | HHHHHHH |
| 299 | 8x his-tag | artificial | HHHHHHHH |
| 300 | SI tag | artificial | NANNPDWDF |
| 301 | S-tag | artificial | KETAAAKFERQHMDS |
| 302 | Strep-tag 1 | artificial | WSHPQFEK |
| 303 | Strep-tag 2 | artificial | AWAHPQPGG |
| 304 | Universal tag | artificial | HTTPHH |
| 305 | VSV-G | artificial | YTDIEMNRLGK |
| 306 | Protein C | artificial | EDQVDPRLIDGK |

1. A method for the prevention or reduction of adverse events associated with immunotherapeutic treatment of cancer with an antibody construct comprising a first domain which binds to a target antigen on the surface of a target cell, and a second domain which binds to human CD3 on the surface of a T cell, wherein said method comprises
(a) administering an antibody construct comprising a first domain which binds to a target antigen on the surface of a target cell, and a second domain which binds to human CD3 on the surface of a T cell,
(b) administering an inhibitor/antagonist of TNF/TNFR that reduces TNF/TNFR signalling,
wherein said inhibitor/antagonist of TNF/TNFR that reduces TNF/TNFR signalling (b) is administered to a patient in need thereof prior to administration of said antibody construct (a), wherein the patient is a human being or a non-human primate.

2. The method for the prevention or reduction of adverse events associated with immunotherapeutic treatment of cancer with an antibody construct comprising a first domain which binds to a target antigen on the surface of a target cell, and a second domain which binds to human CD3 on the surface of a T cell according to claim 1, wherein said method comprises
(a) administering an antibody construct comprising a first domain which binds to a target antigen on the surface of a target cell, and a second domain which binds to human CD3 on the surface of a T cell,
(b) administering an inhibitor/antagonist of TNF/TNFR that reduces TNF/TNFR signalling,
(c) administering a corticosteroid, particularly dexamethasone,
wherein said inhibitor/antagonist of TNF/TNFR that reduces TNF/TNFR signalling in step (b) and said corticosteroid in step (c) are administered to a patient in need thereof prior to administration of said antibody construct (a), wherein the patient is a human being or a non-human primate.

3. The method for the prevention or reduction of adverse events associated with immunotherapeutic treatment of cancer with an antibody construct comprising a first domain which binds to a target antigen on the surface of a target cell, and a second domain which binds to human CD3 on the surface of a T cell according to claim 1 or 2, wherein the adverse event is cytokine release syndrome (CRS).

4. The method for the prevention or reduction of adverse events associated with immunotherapeutic treatment of cancer with an antibody construct comprising a first domain which binds to a target antigen on the surface of a target cell, and a second domain which binds to human CD3 on the surface of a T cell according to any one of claims 1 to 3, wherein the second domain of said antibody construct binds to human CD3 epsilon and to Callithrix jacchus or Saimiri sciureus CD3 epsilon.

5. The method for the prevention or reduction of adverse events associated with immunotherapeutic treatment of cancer with an antibody construct comprising a first domain which binds to a target antigen on the surface of a target cell, and a second domain which binds to human CD3 on the surface of a T cell according to any one of claims 1 to 4, wherein
a) the antibody construct is a single chain antibody construct,
b) the first domain is in the format of an scFv,
c) the second domain is in the format of an scFv,
d) the first and the second domain are connected via a linker, and/or
e) the antibody construct comprises a domain providing an extended serum half-life.

6. The method for the prevention or reduction of adverse events associated with immunotherapeutic treatment of cancer with an antibody construct comprising a first domain which binds to a target antigen on the surface of a target cell, and a second domain which binds to human CD3 on the surface of a T cell according to any one of claims 1 to 5, wherein the first domain binds to a target antigen selected from the group comprising tumor-associated antigens, viral antigens, bacterial antigens, peptide-MHC complexes presenting a peptide fragments derived from a tumor-associated antigen, viral antigen, bacterial antigen, or a disease-associated antigen.

7. The method for the prevention or reduction of adverse events associated with immunotherapeutic treatment of cancer with an antibody construct comprising a first domain which binds to a target antigen on the surface of a target cell, and a second domain which binds to human CD3 on the surface of a T cell according to any one of claims 1 to 6, wherein the first domain binds to a target antigen, wherein said target antigen is a tumor-associated antigen selected from the group comprising CD19, CD33, FLT3, PSMA, BCMA, and DLL3.

8. The method for the prevention or reduction of adverse events associated with immunotherapeutic treatment of cancer with an antibody construct comprising a first domain which binds to a target antigen on the surface of a target cell, and a second domain which binds to human CD3 on the surface of a T cell according to any one of claims 1 to 7, wherein the inhibitor/antagonist of TNF/TNFR that reduces TNF/TNFR signalling is selected from the group comprising small molecules, aptamers, biological molecules, antibodies and derivatives thereof.

9. The method for the prevention or reduction of adverse events associated with immunotherapeutic treatment of cancer with an antibody construct comprising a first domain which binds to a target antigen on the surface of a target cell, and a second domain which binds to human CD3 on the surface of a T cell according to any one of claims 1 to 8, wherein the inhibitor/antagonist of TNF/TNFR that reduces TNF/TNFR signalling is selected from the group comprising etanercept, infliximab, adalimumab, certolizumab Pergol, and golimumab, particularly etanercept.

10. The method for the prevention or reduction of adverse events associated with immunotherapeutic treatment of cancer with an antibody construct comprising a first domain which binds to a target antigen on the surface of a target cell, and a second domain which binds to human CD3 on the surface of a T cell of a disease according to any one of claims 1 to 9, wherein said disease is either a solid tumor or a hematological cancer.

11. The method for the prevention or reduction of adverse events associated with immunotherapeutic treatment of cancer with an antibody construct comprising a first domain which binds to a target antigen on the surface of a target cell, and a second domain which binds to human CD3 on the surface of a T cell according to any one of claims 1 to 10, preferably for immunotherapy of a neoplastic disease, wherein said corticosteroid is dexamethasone provided said patient is neither at risk of developing adverse events nor has an intolerance to dexamethasone, and particularly wherein the inhibitor/antagonist of TNF/TNFR that reduces TNF/TNFR signalling etanercept is particularly preferred.

12. A method for the immunotherapeutic treatment of cancer in a patient, wherein said method comprises
(a) administering an antibody construct comprising a first domain which binds to a target antigen on the surface of a target cell, and a second domain which binds to human CD3 on the surface of a T cell,
(b) administering an inhibitor/antagonist of TNF/TNFR that reduces TNF/TNFR signalling,
wherein said inhibitor/antagonist of TNF/TNFR that reduces TNF/TNFR signalling (b) is administered to a patient in need thereof prior to administration of said antibody construct (a), wherein the patient is a human being or a non-human primate.

13. A method for the immunotherapeutic treatment of cancer in a patient, wherein said method comprises
(a) administering an antibody construct comprising a first domain which binds to a target antigen on the surface of a target cell, and a second domain which binds to human CD3 on the surface of a T cell,
(b) administering an inhibitor/antagonist of TNF/TNFR that reduces TNF/TNFR signalling,
(c) administering a corticosteroid, particularly dexamethasone,
wherein said inhibitor/antagonist of TNF/TNFR that reduces TNF/TNFR signalling in step (b) and the corticosteroid in step (c) administered to a patient in need thereof prior to administration of said antibody construct (a), wherein the patient is a human being or a non-human primate.

14. The method for the immunotherapeutic treatment of cancer in a patient according to claims 12 and 13, wherein the second domain of said antibody construct binds to human CD3 epsilon and to Callithrix jacchus or Saimiri sciureus CD3 epsilon.

15. The method for the immunotherapeutic treatment of cancer in a patient according to any one of claims 12 to 14, wherein
a) the antibody construct is a single chain antibody construct,
b) the first domain is in the format of an scFv,
c) the second domain is in the format of an scFv,
d) the first and the second domain are connected via a linker, and/or
e) the antibody construct comprises a domain providing an extended serum half-life.

16. The method for the immunotherapeutic treatment of cancer in a patient according to any one of claims 12 to 15, wherein the first domain binds to a target antigen selected from the group comprising tumor-associated antigens, viral antigens, bacterial antigens, peptide-MHC complexes presenting a peptide fragments derived from a tumor-associated antigen, viral antigen, bacterial antigen, or a disease-associated antigen.

17. The method for the immunotherapeutic treatment of cancer in a patient according to any one of claims 12 to 16, wherein the first domain binds to a target antigen, wherein said target antigen is a tumor-associated antigen selected from the group comprising CD19, CD33, FLT3, PSMA, BCMA, and DLL3.

18. The method for the immunotherapeutic treatment of cancer in a patient according to any one of claims 12 to 17, wherein the inhibitor/antagonist of TNF/TNFR that reduces TNF/TNFR signalling is selected from the group comprising small molecules, biological molecules, aptamers, antibodies and derivatives thereof.

19. The method for the immunotherapeutic treatment of cancer in a patient according to any one of claims 12 to 18, wherein the inhibitor/antagonist of TNF/TNFR that reduces TNF/TNFR signalling is selected from the group comprising etanercept, infliximab, adalimumab, certolizumab Pergol, and golimumab, particularly etanercept.

20. The method for the immunotherapeutic treatment of cancer in a patient according to any one of claims 12 to 19, wherein the cancer is selected from a solid tumor or a hematological cancer.

21. The method for the immunotherapeutic treatment of cancer in a patient according to any one of claims 12 to 20, wherein said patient is at risk of developing adverse events, or wherein the patient has an intolerance to IL-6R-antagonists, wherein the IL-6R-antagonist is tocilizumab.

22. A combination product for the prevention or reduction of adverse events associated with immunotherapy with an antibody construct comprising a first domain which binds to a target antigen on the surface of a target cell, and a second domain which binds to human CD3 on the surface of a T cell comprising
(a) an antibody construct comprising a first domain which binds to a target antigen on the surface of a target cell, and a second domain which binds to human CD3 on the surface of a T cell,
(b) an inhibitor/antagonist of TNF/TNFR that reduces TNF/TNFR signalling,
wherein said inhibitor/antagonist of TNF/TNFR that reduces TNF/TNFR signalling (b) is for administration to a patient in need thereof prior to administration of said antibody construct (a), wherein said patient is either a human being or a non-human primate.

23. A combination product for the prevention or reduction of adverse events associated with immunotherapy with an antibody construct comprising a first domain which binds to a target antigen on the surface of a target cell, and a second domain which binds to human CD3 on the surface of a T cell comprising
(a) an antibody construct comprising a first domain which binds to a target antigen on the surface of a target cell, and a second domain which binds to human CD3 on the surface of a T cell,
(b) an inhibitor/antagonist of TNF/TNFR that reduces TNF/TNFR signalling,
(c) a corticosteroid, particularly dexamethasone,
wherein said inhibitor/antagonist of TNF/TNFR that reduces TNF/TNFR signalling (b) and the corticosteroid (c) are for administration to a patient in need thereof prior to administration of said antibody construct (a), wherein said patient is either a human being or a non-human primate.

24. The combination product according to claims 22 and 23 for the prevention or reduction of adverse events associated with immunotherapy with an antibody construct comprising a first domain which binds to a target antigen on the surface of a target cell, and a second domain which binds to human CD3 on the surface of a T cell, wherein the adverse event associated with immunotherapy is increased cytokine release of TNF, MCP-1, IL-1, and/or IL-6, particularly wherein the adverse event is CRS.

25. The combination product according to any one of claims 22 to 24 for the prevention or reduction of adverse events associated with immunotherapy with an antibody construct comprising a first domain which binds to a target antigen on the surface of a target cell, and a second domain which binds to human CD3 on the surface of a T cell, wherein the second domain of said antibody construct binds to human CD3 epsilon and to Callithrix jacchus or Saimiri sciureus CD3 epsilon.

26. The combination product according to any one of claims 22 to 24 for the prevention or reduction of adverse events associated with immunotherapy with an antibody construct comprising a first domain which binds to a target antigen on the surface of a target cell, and a second domain which binds to human CD3 on the surface of a T cell, wherein
a) the antibody construct is a single chain antibody construct,
b) the first domain is in the format of an scFv,
c) the second domain is in the format of an scFv,
d) the first and the second domain are connected via a linker, and/or
e) the antibody construct comprises a domain providing an extended serum half-life.

27. The combination product according to any one of the preceding claims 22 to 25 for the prevention or reduction of adverse events associated with immunotherapy with an antibody construct comprising a first domain which binds to a target antigen on the surface of a target cell, and a second domain which binds to human CD3 on the surface of a T cell, wherein the first domain binds to a target antigen selected from the group comprising tumor-associated antigens, viral antigens, bacterial antigens, peptide-MHC complexes presenting a peptide fragment derived from a tumor-associated antigen, viral antigen, bacterial antigen, or a disease-associated antigen.

28. The combination product according to any one of the preceding claims 22 to 27 for the prevention or reduction of adverse events associated with immunotherapy with an antibody construct comprising a first domain which binds to a target antigen on the surface of a target cell, and a second domain which binds to human CD3 on the surface of a T cell, wherein the first domain binds to a target antigen, wherein said target antigen is a tumor-associated antigen selected from the group comprising CD19, CD33, FLT3, PSMA, BCMA, and DLL3.

29. The combination product according to any one of the preceding claims 22 to 28 for the prevention or reduction of adverse events associated with immunotherapy with an antibody construct comprising a first domain which binds to a target antigen on the surface of a target cell, and a second domain which binds to human CD3 on the surface of a T cell, wherein the inhibitor/antagonist of TNF/TNFR that reduces TNF/TNFR signalling is selected from the group comprising small molecules, biological molecules, aptamers, antibodies and derivatives thereof.

30. The combination product according to any one of the preceding claims 22 to 29 for the prevention or reduction of adverse events associated with immunotherapy with an antibody construct comprising a first domain which binds to a target antigen on the surface of a target cell, and a second domain which binds to human CD3 on the surface of a T cell, wherein the inhibitor/antagonist of TNF/TNFR that reduces TNF/TNFR signalling is selected from the group comprising etanercept, infliximab, adalimumab, certolizumab Pergol, and golimumab, particularly etanercept.

31. The combination product according to any of the preceding claims 22 to 30 for the prevention or reduction of adverse events associated with immunotherapy of a disease with an antibody construct comprising a first domain which binds to a target antigen on the surface of a target cell, and a second domain which binds to human CD3 on the surface of a T cell, wherein the disease is a neoplastic disease, particularly a solid tumor or a hematological cancer.

32. The combination product according to any of the preceding claims 22 to 31 for the prevention or reduction of adverse events associated with immunotherapy with an antibody construct comprising a first domain which binds to a target antigen on the surface of a target cell, and a second domain which binds to human CD3 on the surface of a T cell, wherein said combination product is for administration to a patient at risk of developing adverse events, or having an intolerance, to IL-6-antagonists or IL-6R-antagonists, particularly wherein the IL-6R-antagonist is tocilizumab.

33. A kit comprising a combination product according to any of the preceding claims 22 to 32 in a package, wherein the combination product is present in a single container, or the components of said combination product are present individually, said kit optionally further comprising at least one of instructions for use, a device for administration of said combination product or components thereof, at least one separately packed medium for reconstitution, a pharmaceutical carrier for at least one of said combination products or components thereof.

34. An inhibitor/antagonist of TNF/TNFR that reduces TNF/TNFR signalling as defined in any of the foregoing claims 22 to 33 for use in a method for the prevention of adverse events associated with immunotherapy with an antibody construct comprising a first domain which binds to a target antigen on the surface of a target cell, and a second domain which binds to human CD3 on the surface of a T cell in a patient suffering from a neoplastic disease, particularly from solid tumor or a hematological cancer.

35. The inhibitor/antagonist of TNF/TNFR that reduces TNF/TNFR signalling as defined in any of the foregoing claims 22 to 34 for use in a method for the prevention of adverse events associated with immunotherapy with an antibody construct comprising a first domain which binds to a target antigen on the surface of a target cell, and a second domain which binds to human CD3 on the surface of a T cell in a patient suffering from a neoplastic disease as defined in the preceding claim 34, wherein said patient is at risk of developing CRS.

36. The inhibitor/antagonist of TNF/TNFR that reduces TNF/TNFR signalling as defined in any of the foregoing claims 22 to 35 for use in a method for the prevention of CRS in a patient suffering from a neoplastic disease who is at risk of developing CRS as defined in the preceding claims 47 and 48, wherein said patient is a person subjected to therapy with an antibody construct comprising a first domain which binds to a target antigen on the surface of a target cell, and a second domain which binds to human CD3 on the surface of a T cell as defined in any of the preceding claims.

37. Use of an inhibitor/antagonist of TNF/TNFR that reduces TNF/TNFR signalling as defined in any of the foregoing claims wherein said inhibitor/antagonist of TNF/TNFR that reduces TNF/TNFR signalling in the preparation of a medicament for the prevention of adverse events associated with immunotherapy with an antibody construct comprising a first domain which binds to a target antigen on the surface of a target cell, and a second domain which binds to human CD3 on the surface of a T cell, particularly for the prevention of CRS in a patient suffering from a neoplastic disease as defined in any of the preceding claims.


SEQUENCE LISTING (307 sequences)

SEQ ID NO: 1 - protein, 105 aa, Homo Sapiens
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 2 - protein, 27 aa, Homo Sapiens
QDGNEEMGGITQTPYKVSISGTTVILT

SEQ ID NO: 3 - protein, 14 aa, Artificial Sequence
  Synthetic
GSSTGAVTSGYYPN

SEQ ID NO: 4 - protein, 7 aa, Artificial Sequence
  Synthetic
GTKFLAP

SEQ ID NO: 5 - protein, 9 aa, Artificial Sequence
  Synthetic
ALWYSNRWV

SEQ ID NO: 6 - protein, 14 aa, Artificial Sequence
  Synthetic
RSSTGAVTSGYYPN

SEQ ID NO: 7 - protein, 7 aa, Artificial Sequence
  Synthetic
ATDMRPS

SEQ ID NO: 8 - protein, 9 aa, Artificial Sequence
  Synthetic
ALWYSNRWV

SEQ ID NO: 9 - protein, 14 aa, Artificial Sequence
  Synthetic
GSSTGAVTSGNYPN

SEQ ID NO: 10 - protein, 7 aa, Artificial Sequence
  Synthetic
GTKFLAP

SEQ ID NO: 11 - protein, 9 aa, Artificial Sequence
  Synthetic
VLWYSNRWV

SEQ ID NO: 12 - protein, 5 aa, Artificial Sequence
  Synthetic
IYAMN

SEQ ID NO: 13 - protein, 19 aa, Artificial Sequence
  Synthetic
RIRSKYNNYATYYADSVKS

SEQ ID NO: 14 - protein, 14 aa, Artificial Sequence
  Synthetic
HGNFGNSYVSFFAY

SEQ ID NO: 15 - protein, 5 aa, Artificial Sequence
  Synthetic
KYAMN

SEQ ID NO: 16 - protein, 19 aa, Artificial Sequence
  Synthetic
RIRSKYNNYATYYADSVKD

SEQ ID NO: 17 - protein, 14 aa, Artificial Sequence
  Synthetic
HGNFGNSYISYWAY

SEQ ID NO: 18 - protein, 5 aa, Artificial Sequence
  Synthetic
SYAMN

SEQ ID NO: 19 - protein, 19 aa, Artificial Sequence
  Synthetic
RIRSKYNNYATYYADSVKG

SEQ ID NO: 20 - protein, 14 aa, Artificial Sequence
  Synthetic
HGNFGNSYLSFWAY

SEQ ID NO: 21 - protein, 5 aa, Artificial Sequence
  Synthetic
RYAMN

SEQ ID NO: 22 - protein, 19 aa, Artificial Sequence
  Synthetic
RIRSKYNNYATYYADSVKG

SEQ ID NO: 23 - protein, 14 aa, Artificial Sequence
  Synthetic
HGNFGNSYLSYFAY

SEQ ID NO: 24 - protein, 5 aa, Artificial Sequence
  Synthetic
VYAMN

SEQ ID NO: 25 - protein, 19 aa, Artificial Sequence
  Synthetic
RIRSKYNNYATYYADSVKK

SEQ ID NO: 26 - protein, 14 aa, Artificial Sequence
  Synthetic
HGNFGNSYLSWWAY

SEQ ID NO: 27 - protein, 5 aa, Artificial Sequence
  Synthetic
KYAMN

SEQ ID NO: 28 - protein, 19 aa, Artificial Sequence
  Synthetic
RIRSKYNNYATYYADSVKS

SEQ ID NO: 29 - protein, 14 aa, Artificial Sequence
  Synthetic
HGNFGNSYTSYYAY

SEQ ID NO: 30 - protein, 5 aa, Artificial Sequence
  Synthetic
GYAMN

SEQ ID NO: 31 - protein, 19 aa, Artificial Sequence
  Synthetic
RIRSKYNNYATYYADSVKE

SEQ ID NO: 32 - protein, 14 aa, Artificial Sequence
  Synthetic
HRNFGNSYLSWFAY

SEQ ID NO: 33 - protein, 5 aa, Artificial Sequence
  Synthetic
VYAMN

SEQ ID NO: 34 - protein, 19 aa, Artificial Sequence
  Synthetic
RIRSKYNNYATYYADSVKK

SEQ ID NO: 35 - protein, 14 aa, Artificial Sequence
  Synthetic
HGNFGNSYISWWAY

SEQ ID NO: 36 - protein, 5 aa, Artificial Sequence
  Synthetic
SYAMN

SEQ ID NO: 37 - protein, 19 aa, Artificial Sequence
  Synthetic
RIRSKYNNYATYYADSVKG

SEQ ID NO: 38 - protein, 14 aa, Artificial Sequence
  Synthetic
HGNFGNSYVSWWAY

SEQ ID NO: 39 - protein, 5 aa, Artificial Sequence
  Synthetic
KYAMN

SEQ ID NO: 40 - protein, 19 aa, Artificial Sequence
  Synthetic
RIRSKYNNYATYYADSVKD

SEQ ID NO: 41 - protein, 14 aa, Artificial Sequence
  Synthetic
HGNFGNSYISYWAY

SEQ ID NO: 42 - protein, 7 aa, Artificial Sequence
  Synthetic
GTKFLAP

SEQ ID NO: 43 - protein, 9 aa, Artificial Sequence
  Synthetic
ALWYSNRWV

SEQ ID NO: 44 - protein, 5 aa, Artificial Sequence
  Synthetic
KYAMN

SEQ ID NO: 45 - protein, 14 aa, Artificial Sequence
  Synthetic
GSSTGAVTSGYYPN

SEQ ID NO: 46 - protein, 7 aa, Artificial Sequence
  Synthetic
GTKFLAP

SEQ ID NO: 47 - protein, 9 aa, Artificial Sequence
  Synthetic
ALWYSNRWV

SEQ ID NO: 48 - protein, 5 aa, Artificial Sequence
  Synthetic
SYAMN

SEQ ID NO: 49 - protein, 19 aa, Artificial Sequence
  Synthetic
RIRSKYNNYATYYADSVKG

SEQ ID NO: 50 - protein, 14 aa, Artificial Sequence
  Synthetic
HGNFGNSYLSFWAY

SEQ ID NO: 51 - protein, 14 aa, Artificial Sequence
  Synthetic
GSSTGAVTSGYYPN

SEQ ID NO: 52 - protein, 7 aa, Artificial Sequence
  Synthetic
GTKFLAP

SEQ ID NO: 53 - protein, 9 aa, Artificial Sequence
  Synthetic
ALWYSNRWV

SEQ ID NO: 54 - protein, 14 aa, Artificial Sequence
  Synthetic
RSSTGAVTSGYYPN

SEQ ID NO: 55 - protein, 7 aa, Artificial Sequence
  Synthetic
ATDMRPS

SEQ ID NO: 56 - protein, 9 aa, Artificial Sequence
  Synthetic
ALWYSNRWV

SEQ ID NO: 57 - protein, 14 aa, Artificial Sequence
  Synthetic
GSSTGAVTSGYYPN

SEQ ID NO: 58 - protein, 7 aa, Artificial Sequence
  Synthetic
GTKFLAP

SEQ ID NO: 59 - protein, 9 aa, Artificial Sequence
  Synthetic
ALWYSNRWV

SEQ ID NO: 60 - protein, 14 aa, Artificial Sequence
  Synthetic
GSSTGAVTSGYYPN

SEQ ID NO: 61 - protein, 7 aa, Artificial Sequence
  Synthetic
GTKFLAP

SEQ ID NO: 62 - protein, 9 aa, Artificial Sequence
  Synthetic
ALWYSNRWV

SEQ ID NO: 63 - protein, 14 aa, Artificial Sequence
  Synthetic
GSSTGAVTSGNYPN

SEQ ID NO: 64 - protein, 7 aa, Artificial Sequence
  Synthetic
GTKFLAP

SEQ ID NO: 65 - protein, 9 aa, Artificial Sequence
  Synthetic
VLWYSNRWV

SEQ ID NO: 66 - protein, 109 aa, Artificial Sequence
  Synthetic
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 67 - protein, 109 aa, Artificial Sequence
  Synthetic
QTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 68 - protein, 109 aa, Artificial Sequence
  Synthetic
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 69 - protein, 109 aa, Artificial Sequence
  Synthetic
ELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 70 - protein, 109 aa, Artificial Sequence
  Synthetic
ELVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 71 - protein, 109 aa, Artificial Sequence
  Synthetic
ELVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 72 - protein, 125 aa, Artificial Sequence
  Synthetic
EVQLVESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSS

SEQ ID NO: 73 - protein, 125 aa, Artificial Sequence
  Synthetic
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 74 - protein, 125 aa, Artificial Sequence
  Synthetic
EVQLVESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSS

SEQ ID NO: 75 - protein, 125 aa, Artificial Sequence
  Synthetic
EVQLVESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSS

SEQ ID NO: 76 - protein, 125 aa, Artificial Sequence
  Synthetic
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSS

SEQ ID NO: 77 - protein, 125 aa, Artificial Sequence
  Synthetic
EVQLVESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSS

SEQ ID NO: 78 - protein, 125 aa, Artificial Sequence
  Synthetic
EVQLVESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSS

SEQ ID NO: 79 - protein, 125 aa, Artificial Sequence
  Synthetic
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSS

SEQ ID NO: 80 - protein, 125 aa, Artificial Sequence
  Synthetic
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSS

SEQ ID NO: 81 - protein, 125 aa, Artificial Sequence; identical to SEQ ID NO: 73
  Synthetic

SEQ ID NO: 82 - protein, 125 aa, Artificial Sequence
  Synthetic
EVQLVESGGGLVQPGGSLRLSCAASGFTFNSYAMNWVRQAPGKGLEWVARIRSKYNNYATYYADSVKGRFTISRDDSKNTAYLQMNSLKTEDTAVYYCVRHGNFGNSYVSWWAYWGQGTLVTVSS

SEQ ID NO: 83 - protein, 125 aa, Artificial Sequence
  Synthetic
EVQLLESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSS

SEQ ID NO: 84 - protein, 125 aa, Artificial Sequence
  Synthetic
EVQLLESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 85 - protein, 125 aa, Artificial Sequence
  Synthetic
EVQLLESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSS

SEQ ID NO: 86 - protein, 125 aa, Artificial Sequence
  Synthetic
EVQLLESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSS

SEQ ID NO: 87 - protein, 125 aa, Artificial Sequence
  Synthetic
EVQLLESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSS

SEQ ID NO: 88 - protein, 125 aa, Artificial Sequence
  Synthetic
EVQLLESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSS

SEQ ID NO: 89 - protein, 125 aa, Artificial Sequence
  Synthetic
EVQLLESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSS

SEQ ID NO: 90 - protein, 125 aa, Artificial Sequence
  Synthetic
EVQLLESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSS

SEQ ID NO: 91 - protein, 125 aa, Artificial Sequence
  Synthetic
EVQLLESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSS

SEQ ID NO: 92 - protein, 125 aa, Artificial Sequence; identical to SEQ ID NO: 84
  Synthetic

SEQ ID NO: 93 - protein, 109 aa, Artificial Sequence; identical to SEQ ID NO: 66
  Synthetic

SEQ ID NO: 94 - protein, 109 aa, Artificial Sequence; identical to SEQ ID NO: 66
  Synthetic

SEQ ID NO: 95 - protein, 109 aa, Artificial Sequence; identical to SEQ ID NO: 66
  Synthetic

SEQ ID NO: 96 - protein, 109 aa, Artificial Sequence; identical to SEQ ID NO: 67
  Synthetic

SEQ ID NO: 97 - protein, 109 aa, Artificial Sequence; identical to SEQ ID NO: 66
  Synthetic

SEQ ID NO: 98 - protein, 109 aa, Artificial Sequence; identical to SEQ ID NO: 66
  Synthetic

SEQ ID NO: 99 - protein, 109 aa, Artificial Sequence; identical to SEQ ID NO: 68
  Synthetic

SEQ ID NO: 100 - protein, 109 aa, Artificial Sequence; identical to SEQ ID NO: 68
  Synthetic

SEQ ID NO: 101 - protein, 249 aa, Artificial Sequence
  Synthetic
EVQLVESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 102 - protein, 249 aa, Artificial Sequence
  Synthetic
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 103 - protein, 249 aa, Artificial Sequence
  Synthetic
EVQLVESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 104 - protein, 249 aa, Artificial Sequence
  Synthetic
EVQLVESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 105 - protein, 249 aa, Artificial Sequence
  Synthetic
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSSGGGGSGGGGSGGGGSQTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 106 - protein, 249 aa, Artificial Sequence
  Synthetic
EVQLVESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 107 - protein, 249 aa, Artificial Sequence
  Synthetic
EVQLVESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSSGGGGSGGGGSGGGGSQTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 108 - protein, 249 aa, Artificial Sequence
  Synthetic
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 109 - protein, 249 aa, Artificial Sequence
  Synthetic
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 110 - protein, 249 aa, Artificial Sequence
  Synthetic
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 111 - protein, 249 aa, Artificial Sequence
  Synthetic
EVQLVESGGGLVQPGGSLRLSCAASGFTFNSYAMNWVRQAPGKGLEWVARIRSKYNNYATYYADSVKGRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 112 - protein, 249 aa, Artificial Sequence
  Synthetic
EVQLLESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSSGGGGSGGGGSGGGGSELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 113 - protein, 249 aa, Artificial Sequence
  Synthetic
EVQLLESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 114 - protein, 249 aa, Artificial Sequence
  Synthetic
EVQLLESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSSGGGGSGGGGSGGGGSELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 115 - protein, 249 aa, Artificial Sequence
  Synthetic
EVQLLESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSSGGGGSGGGGSGGGGSELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 116 - protein, 249 aa, Artificial Sequence
  Synthetic
EVQLLESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSSGGGGSGGGGSGGGGSELVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 117 - protein, 249 aa, Artificial Sequence
  Synthetic
EVQLLESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSSGGGGSGGGGSGGGGSELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 118 - protein, 249 aa, Artificial Sequence
  Synthetic
EVQLLESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSSGGGGSGGGGSGGGGSELVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 119 - protein, 249 aa, Artificial Sequence
  Synthetic
EVQLLESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSSGGGGSGGGGSGGGGSELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 120 - protein, 249 aa, Artificial Sequence
  Synthetic
EVQLLESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSELVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 121 - protein, 249 aa, Artificial Sequence
  Synthetic
EVQLLESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSELVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 122 - protein, 5 aa, Artificial Sequence
  Synthetic
NYGMH

SEQ ID NO: 123 - protein, 17 aa, Artificial Sequence
  Synthetic
LIWYDGSKKYYADSVKG

SEQ ID NO: 124 - protein, 16 aa, Artificial Sequence
  Synthetic
DPSSLTGSTGYYGMDV

SEQ ID NO: 125 - protein, 11 aa, Artificial Sequence
  Synthetic
SGDKLGDKYAC

SEQ ID NO: 126 - protein, 7 aa, Artificial Sequence
  Synthetic
QDRKRPS

SEQ ID NO: 127 - protein, 9 aa, Artificial Sequence
  Synthetic
QAWDSSTEV

SEQ ID NO: 128 - protein, 231 aa, Artificial Sequence
  Synthetic
MAWALLLLTLLTQGTGSWASYELTQPPSVSVSPGQTASITCSGDKLGDKYACWYQQKPGQSPVLVIYQDRKRPSGIPERFSGSNSGNTATLTISGTQAMDEADYYCQAWDSSTEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 129 - protein, 5 aa, Artificial Sequence
  Synthetic
SYDMS

SEQ ID NO: 130 - protein, 17 aa, Artificial Sequence
  Synthetic
LISGGGSNTYYADSVKG

SEQ ID NO: 131 - protein, 11 aa, Artificial Sequence
  Synthetic
PSGHYFYAMDV

SEQ ID NO: 132 - protein, 11 aa, Artificial Sequence
  Synthetic
RASQGISNWLA

SEQ ID NO: 133 - protein, 7 aa, Artificial Sequence
  Synthetic
AASSLQS

SEQ ID NO: 134 - protein, 5 aa, Artificial Sequence
  Synthetic
DYSMS

SEQ ID NO: 135 - protein, 17 aa, Artificial Sequence
  Synthetic
GINWNGGRTRYADSVKG

SEQ ID NO: 136 - protein, 12 aa, Artificial Sequence
  Synthetic
EFNNFESNWFDP

SEQ ID NO: 137 - protein, 11 aa, Artificial Sequence
  Synthetic
SGDALPKKYAY

SEQ ID NO: 138 - protein, 7 aa, Artificial Sequence
  Synthetic
EDSKRPS

SEQ ID NO: 139 - protein, 11 aa, Artificial Sequence
  Synthetic
YSTDSSGNHRV

SEQ ID NO: 140 - protein, 5 aa, Artificial Sequence
  Synthetic
SYDMS

SEQ ID NO: 141 - protein, 17 aa, Artificial Sequence
  Synthetic
LISGGGSNTYYAESVKG

SEQ ID NO: 142 - protein, 11 aa, Artificial Sequence
  Synthetic
PSGHYFYAMDV

SEQ ID NO: 143 - protein, 11 aa, Artificial Sequence
  Synthetic
RASQGISNWLA

SEQ ID NO: 144 - protein, 7 aa, Artificial Sequence
  Synthetic
AASSLQS

SEQ ID NO: 145 - protein, 9 aa, Artificial Sequence
  Synthetic
QQAESFPHT

SEQ ID NO: 146 - protein, 5 aa, Artificial Sequence
  Synthetic
SYDMS

SEQ ID NO: 147 - protein, 17 aa, Artificial Sequence
  Synthetic
LISGGGSQTYYAESVKG

SEQ ID NO: 148 - protein, 11 aa, Artificial Sequence
  Synthetic
PSGHYFYAMDV

SEQ ID NO: 149 - protein, 11 aa, Artificial Sequence
  Synthetic
RASQGISNWLA

SEQ ID NO: 150 - protein, 7 aa, Artificial Sequence
  Synthetic
AASSLQS

SEQ ID NO: 151 - protein, 9 aa, Artificial Sequence
  Synthetic
QQAESFPHT

SEQ ID NO: 152 - protein, 5 aa, Artificial Sequence
  Synthetic
NYGMH

SEQ ID NO: 153 - protein, 17 aa, Artificial Sequence
  Synthetic
LIWYDASKKYYAESVKG

SEQ ID NO: 154 - protein, 16 aa, Artificial Sequence
  Synthetic
DPSSLTGSTGYYGMDV

SEQ ID NO: 155 - protein, 11 aa, Artificial Sequence
  Synthetic
SGDKLGDKYAS

SEQ ID NO: 156 - protein, 7 aa, Artificial Sequence
  Synthetic
QDRKRPS

SEQ ID NO: 157 - protein, 9 aa, Artificial Sequence
  Synthetic
QAFESSTEV

SEQ ID NO: 158 - protein, 5 aa, Artificial Sequence
  Synthetic
DYSMS

SEQ ID NO: 159 - protein, 17 aa, Artificial Sequence
  Synthetic
GINWNGGRTRYADAVKG

SEQ ID NO: 160 - protein, 12 aa, Artificial Sequence
  Synthetic
EFNNFESNWFDP

SEQ ID NO: 161 - protein, 11 aa, Artificial Sequence
  Synthetic
SGDALPKKYAY

SEQ ID NO: 162 - protein, 7 aa, Artificial Sequence
  Synthetic
EDAKRPS

SEQ ID NO: 163 - protein, 11 aa, Artificial Sequence
  Synthetic
YSTDASGNHRV

SEQ ID NO: 164 - protein, 5 aa, Artificial Sequence
  Synthetic
DYSMS

SEQ ID NO: 165 - protein, 17 aa, Artificial Sequence
  Synthetic
GINWNAGRTRYADAVKG

SEQ ID NO: 166 - protein, 12 aa, Artificial Sequence
  Synthetic
EFNNFESNWFDP

SEQ ID NO: 167 - protein, 11 aa, Artificial Sequence
  Synthetic
SGDALPKKYAY

SEQ ID NO: 168 - protein, 7 aa, Artificial Sequence
  Synthetic
EDAKRPS

SEQ ID NO: 169 - protein, 11 aa, Artificial Sequence
  Synthetic
YSTDASGNHRV

SEQ ID NO: 170 - protein, 125 aa, Artificial Sequence
  Synthetic
QVQLVESGGGVVQPGRSLRLSCAASGFTFSNYGMHWVRQAPGKGLEWVALIWYDGSKKYYADSVKGRFTISRDNSKNTLYLQMNSLRAEDTAAYYCARDPSSLTGSTGYYGMDVWGQGTTVTVSS

SEQ ID NO: 171 - protein, 106 aa, Artificial Sequence
  Synthetic
SYELTQPPSVSVSPGQTASITCSGDKLGDKYACWYQQKPGQSPVLVIYQDRKRPSGIPERFSGSNSGNTATLTISGTQAMDEADYYCQAWDSSTEVFGGGTKLTVL

SEQ ID NO: 172 - protein, 120 aa, Artificial Sequence
  Synthetic
EVQLLESGGGLVQPGGALRLSCAASGFTFSSYDMSWVRQAPGKGLEWVSLISGGGSNTYYADSVKGRFTISRDNSKNTLYLQMNSLRAEDTAVYFCASPSGHYFYAMDVWGQGTTVTVSS

SEQ ID NO: 173 - protein, 107 aa, Artificial Sequence
  Synthetic
DIQMTQSPSSVSASVGDRVTITCRASQGISNWLAWYQQKPGKAPKLLIFAASSLQSGVPSRFGGSGSGTDFTFTISSLQPEDFATYYCQQADSFPHTFGGGTKVEIK

SEQ ID NO: 174 - protein, 121 aa, Artificial Sequence
  Synthetic
EVQLVESGGSVVRPGGSLRLSCAASGFTVDDYSMSWVRQVPGKGLEWVSGINWNGGRTRYADSVKGRFTISRDSAKNSLYLQMNSLRAEDTALYYCAREFNNFESNWFDPWGQGTLVTVSS

SEQ ID NO: 175 - protein, 108 aa, Artificial Sequence
  Synthetic
SYELTQPPSVSVSPGQTARITCSGDALPKKYAYWYQQKSGQAPVLVISEDSKRPSGIPERFSGSSSGTMATLTISGAQVEDEADYYCYSTDSSGNHRVFGGGTKLTVL

SEQ ID NO: 176 - protein, 120 aa, Artificial Sequence
  Synthetic
EVQLLESGGGLVQPGGSLRLSCAASGFTFSSYDMSWVRQAPGKGLEWVSLISGGGSNTYYAESVKGRFTISRDNSKNTLYLQMNSLRAEDTAVYFCASPSGHYFYAMDVWGQGTTVTVSS

SEQ ID NO: 177 - protein, 107 aa, Artificial Sequence
  Synthetic
DIQMTQSPSSVSASVGDRVTITCRASQGISNWLAWYQQKPGKAPKLLIFAASSLQSGVPSRFSGSGSGTDFTLTISSLQPEDFATYYCQQAESFPHTFGGGTKVEIK

SEQ ID NO: 178 - protein, 120 aa, Artificial Sequence
  Synthetic
EVQLLESGGGLVQPGGSLRLSCAASGFTFSSYDMSWVRQAPGKGLEWVSLISGGGSQTYYAESVKGRFTISRDNSKNTLYLQMNSLRAEDTAVYFCASPSGHYFYAMDVWGQGTTVTVSS

SEQ ID NO: 179 - protein, 107 aa, Artificial Sequence; identical to SEQ ID NO: 177
  Synthetic

SEQ ID NO: 180 - protein, 125 aa, Artificial Sequence
  Synthetic
QVQLVESGGGVVQPGRSLRLSCAASGFTFSNYGMHWVRQAPGKGLEWVALIWYDASKKYYAESVKGRFTISRDNSKNTLYLQMNSLRAEDTAAYYCARDPSSLTGSTGYYGMDVWGQGTTVTVSS

SEQ ID NO: 181 - protein, 106 aa, Artificial Sequence
  Synthetic
SYELTQPPSVSVSPGQTASITCSGDKLGDKYASWYQQKPGQSPVLVIYQDRKRPSGIPERFSGSNSGNTATLTISGTQAMDEADYYCQAFESSTEVFGGGTKLTVL

SEQ ID NO: 182 - protein, 121 aa, Artificial Sequence
  Synthetic
EVQLVESGGSVVRPGGSLRLSCAASGFTVDDYSMSWVRQVPGKGLEWVSGINWNGGRTRYADAVKGRFTISRDSAKNSLYLQMNSLRAEDTALYYCAREFNNFESNWFDPWGQGTLVTVSS

SEQ ID NO: 183 - protein, 108 aa, Artificial Sequence
  Synthetic
SYELTQPPSVSVSPGQTARITCSGDALPKKYAYWYQQKPGQAPVLVISEDAKRPSGIPERFSGSSSGTMATLTISGAQVEDEADYYCYSTDASGNHRVFGGGTKLTVL

SEQ ID NO: 184 - protein, 121 aa, Artificial Sequence
  Synthetic
EVQLVESGGSVVRPGGSLRLSCAASGFTVDDYSMSWVRQVPGKGLEWVSGINWNAGRTRYADAVKGRFTISRDSAKNSLYLQMNSLRAEDTALYYCAREFNNFESNWFDPWGQGTLVTVSS

SEQ ID NO: 185 - protein, 108 aa, Artificial Sequence; identical to SEQ ID NO: 183
  Synthetic

SEQ ID NO: 186 - protein, 477 aa, Artificial Sequence
  Synthetic
MDMRVPAQLLGLLLLWLRGARCQVQLVESGGGVVQPGRSLRLSCAASGFTFSNYGMHWVRQAPGKGLEWVALIWYDGSKKYYADSVKGRFTISRDNSKNTLYLQMNSLRAEDTAAYYCARDPSSLTGSTGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 231 aa, Artificial Sequence; identical to SEQ ID NO: 128
  Synthetic

SEQ ID NO: 188 - protein, 472 aa, Artificial Sequence
  Synthetic
MDMRVPAQLLGLLLLWLRGARCEVQLLESGGGLVQPGGALRLSCAASGFTFSSYDMSWVRQAPGKGLEWVSLISGGGSNTYYADSVKGRFTISRDNSKNTLYLQMNSLRAEDTAVYFCASPSGHYFYAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - protein, 236 aa, Artificial Sequence
  Synthetic
MDMRVPAQLLGLLLLWLRGARCDIQMTQSPSSVSASVGDRVTITCRASQGISNWLAWYQQKPGKAPKLLIFAASSLQSGVPSRFGGSGSGTDFTFTISSLQPEDFATYYCQQADSFPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 190 - protein, 473 aa, Artificial Sequence
  Synthetic
MDMRVPAQLLGLLLLWLRGARCEVQLVESGGSVVRPGGSLRLSCAASGFTVDDYSMSWVRQVPGKGLEWVSGINWNGGRTRYADSVKGRFTISRDSAKNSLYLQMNSLRAEDTALYYCAREFNNFESNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 233 aa, Artificial Sequence
  Synthetic
MAWALLLLTLLTQGTGSWASYELTQPPSVSVSPGQTARITCSGDALPKKYAYWYQQKSGQAPVLVISEDSKRPSGIPERFSGSSSGTMATLTISGAQVEDEADYYCYSTDSSGNHRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 192 - protein, 472 aa, Artificial Sequence
  Synthetic
MDMRVPAQLLGLLLLWLRGARCEVQLLESGGGLVQPGGSLRLSCAASGFTFSSYDMSWVRQAPGKGLEWVSLISGGGSNTYYAESVKGRFTISRDNSKNTLYLQMNSLRAEDTAVYFCASPSGHYFYAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - protein, 236 aa, Artificial Sequence
  Synthetic
MDMRVPAQLLGLLLLWLRGARCDIQMTQSPSSVSASVGDRVTITCRASQGISNWLAWYQQKPGKAPKLLIFAASSLQSGVPSRFSGSGSGTDFTLTISSLQPEDFATYYCQQAESFPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 194 - protein, 472 aa, Artificial Sequence
  Synthetic
MDMRVPAQLLGLLLLWLRGARCEVQLLESGGGLVQPGGSLRLSCAASGFTFSSYDMSWVRQAPGKGLEWVSLISGGGSQTYYAESVKGRFTISRDNSKNTLYLQMNSLRAEDTAVYFCASPSGHYFYAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - protein, 236 aa, Artificial Sequence; identical to SEQ ID NO: 193
  Synthetic

SEQ ID NO: 196 - protein, 477 aa, Artificial Sequence
  Synthetic
MDMRVPAQLLGLLLLWLRGARCQVQLVESGGGVVQPGRSLRLSCAASGFTFSNYGMHWVRQAPGKGLEWVALIWYDASKKYYAESVKGRFTISRDNSKNTLYLQMNSLRAEDTAAYYCARDPSSLTGSTGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - protein, 231 aa, Artificial Sequence
  Synthetic
MAWALLLLTLLTQGTGSWASYELTQPPSVSVSPGQTASITCSGDKLGDKYASWYQQKPGQSPVLVIYQDRKRPSGIPERFSGSNSGNTATLTISGTQAMDEADYYCQAFESSTEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 198 - protein, 473 aa, Artificial Sequence
  Synthetic
MDMRVPAQLLGLLLLWLRGARCEVQLVESGGSVVRPGGSLRLSCAASGFTVDDYSMSWVRQVPGKGLEWVSGINWNGGRTRYADAVKGRFTISRDSAKNSLYLQMNSLRAEDTALYYCAREFNNFESNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - protein, 233 aa, Artificial Sequence
  Synthetic
MAWALLLLTLLTQGTGSWASYELTQPPSVSVSPGQTARITCSGDALPKKYAYWYQQKPGQAPVLVISEDAKRPSGIPERFSGSSSGTMATLTISGAQVEDEADYYCYSTDASGNHRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 200 - protein, 473 aa, Artificial Sequence
  Synthetic
MDMRVPAQLLGLLLLWLRGARCEVQLVESGGSVVRPGGSLRLSCAASGFTVDDYSMSWVRQVPGKGLEWVSGINWNAGRTRYADAVKGRFTISRDSAKNSLYLQMNSLRAEDTALYYCAREFNNFESNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 201 - protein, 233 aa, Artificial Sequence; identical to SEQ ID NO: 199
  Synthetic

SEQ ID NO: 202 - protein, 4 aa, Artificial Sequence
  Synthetic
GGGG

SEQ ID NO: 203 - protein, 5 aa, Artificial Sequence
  Synthetic
GGGGS

SEQ ID NO: 204 - protein, 5 aa, Artificial Sequence
  Synthetic
GGGGQ

SEQ ID NO: 205 - protein, 6 aa, Artificial Sequence
  Synthetic
SGGGGS

SEQ ID NO: 206 - protein, 6 aa, Artificial Sequence
  Synthetic
PGGGGS

SEQ ID NO: 207 - protein, 6 aa, Artificial Sequence
  Synthetic
PGGDGS

SEQ ID NO: 208 - protein, 9 aa, Artificial Sequence
  Synthetic
GGGGSGGGS

SEQ ID NO: 209 - protein, 10 aa, Artificial Sequence
  Synthetic
GGGGSGGGGS

SEQ ID NO: 210 - protein, 15 aa, Artificial Sequence
  Synthetic
GGGGSGGGGSGGGGS

SEQ ID NO: 211 - protein, 20 aa, Artificial Sequence
  Synthetic
GGGGSGGGGSGGGGSGGGGS

SEQ ID NO: 212 - protein, 25 aa, Artificial Sequence
  Synthetic
GGGGSGGGGSGGGGSGGGGSGGGGS

SEQ ID NO: 213 - protein, 30 aa, Artificial Sequence
  Synthetic
GGGGSGGGGSGGGGSGGGGSGGGGSGGGGS

SEQ ID NO: 214 - protein, 35 aa, Artificial Sequence
  Synthetic
GGGGSGGGGSGGGGSGGGGSGGGGSGGGGSGGGGS

SEQ ID NO: 215 - protein, 40 aa, Artificial Sequence
  Synthetic
GGGGSGGGGSGGGGSGGGGSGGGGSGGGGSGGGGSGGGGS

SEQ ID NO: 216 - protein, 16 aa, Artificial Sequence
  Synthetic
RDWDFDVFGGGTPVGG

SEQ ID NO: 217 - protein, 16 aa, Artificial Sequence
  Synthetic
  misc_feature (12)..(12): Xaa can be any naturally occurring amino acid
QRFVTGHFGGLXPANG

SEQ ID NO: 218 - protein, 16 aa, Artificial Sequence
  Synthetic
QRFVTGHFGGLYPANG

SEQ ID NO: 219 - protein, 16 aa, Artificial Sequence
  Synthetic
QRFVTGHFGGLHPANG

SEQ ID NO: 220 - protein, 9 aa, Artificial Sequence
  Synthetic
TGHFGGLHP

SEQ ID NO: 221 - protein, 16 aa, Artificial Sequence
  Synthetic
QRFCTGHFGGLHPCNG

SEQ ID NO: 222 - protein, 585 aa, Homo Sapiens [sequence omitted]

SEQ ID NO: 223 - protein, 585 aa, Artificial Sequence
  Synthetic
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAGTFTFHADICTLSEKERQIKKQTALVELVKHKPKATKEQLKAAMDDFAAFVEKCCKADDKETCFAEEGKKLVAASQAALGL

SEQ ID NO: 224 - protein, 585 aa, Artificial Sequence
  Synthetic
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 225 - protein, 585 aa, Artificial Sequence
  Synthetic
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 226 - protein, 585 aa, Artificial Sequence
  Synthetic
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALGVDETYVPKEFNAETFTFHADICTLSEKERQIKKQTALVELVKHKPKATKEQLKAVMDKFAAFVEKCCKADDKETCFAEEGPKLVAASQAALGL

SEQ ID NO: 227 - protein, 585 aa, Artificial Sequence
  Synthetic
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDKFAAFVEKCCKADDKETCFAEEGPKLVAASQAALGL

SEQ ID NO: 228 - protein, 585 aa, Artificial Sequence
  Synthetic
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDKFAAFVEKCCKADDKETCFAEEGPHLVAASQAALGL

SEQ ID NO: 229 - protein, 585 aa, Artificial Sequence
  Synthetic
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 230 - protein, 585 aa, Artificial Sequence
  Synthetic
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDKFAAFVEKCCKADDKETCFAEEGPKLVAASKAALGL

SEQ ID NO: 231 - protein, 585 aa, Artificial Sequence
  Synthetic
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDDFAAFVEKCCKADDKETCFAEEGPKLVAASKAALGL

SEQ ID NO: 232 - protein, 585 aa, Artificial Sequence
  Synthetic
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 233 - protein, 585 aa, Artificial Sequence
  Synthetic
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAGTFTFHADICTLSEKERQIKKQTALVELVKHKPKATKEQLKAAMDDFAAFVEKCCKADDKETCFAEEGKKLVAASQAALGL

SEQ ID NO: 234 - protein, 585 aa, Artificial Sequence
  Synthetic
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 235 - protein, 585 aa, Artificial Sequence
  Synthetic
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 236 - protein, 585 aa, Artificial Sequence
  Synthetic
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALGVDETYVPKEFNAETFTFHADICTLSEKERQIKKQTALVELVKHKPKATKEQLKAVMDKFAAFVEKCCKADDKETCFAEEGPKLVAASQAALGL

SEQ ID NO: 237 - protein, 585 aa, Artificial Sequence
  Synthetic
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDKFAAFVEKCCKADDKETCFAEEGPKLVAASQAALGL

SEQ ID NO: 238 - protein, 585 aa, Artificial Sequence
  Synthetic
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDKFAAFVEKCCKADDKETCFAEEGPHLVAASQAALGL

SEQ ID NO: 239 - protein, 585 aa, Artificial Sequence
  Synthetic
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 240 - protein, 585 aa, Artificial Sequence
  Synthetic
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDKFAAFVEKCCKADDKETCFAEEGPKLVAASKAALGL

SEQ ID NO: 241 - protein, 585 aa, Artificial Sequence
  Synthetic
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDDFAAFVEKCCKADDKETCFAEEGPKLVAASKAALGL

SEQ ID NO: 242 - protein, 585 aa, Artificial Sequence
  Synthetic
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 243 - protein, 585 aa, Artificial Sequence
  Synthetic
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAGTFTFHADICTLSEKERQIKKQTALVELVKHKPKATKEQLKAAMDDFAAFVEKCCKADDKETCFAEEGKKLVAASQAALGL

SEQ ID NO: 244 - protein, 585 aa, Artificial Sequence
  Synthetic
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 245 - protein, 585 aa, Artificial Sequence
  Synthetic
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 246 - protein, 585 aa, Artificial Sequence
  Synthetic
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALGVDETYVPKEFNAETFTFHADICTLSEKERQIKKQTALVELVKHKPKATKEQLKAVMDKFAAFVEKCCKADDKETCFAEEGPKLVAASQAALGL

SEQ ID NO: 247 - protein, 585 aa, Artificial Sequence
  Synthetic
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDKFAAFVEKCCKADDKETCFAEEGPKLVAASQAALGL

SEQ ID NO: 248 - protein, 585 aa, Artificial Sequence
  Synthetic
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDKFAAFVEKCCKADDKETCFAEEGPHLVAASQAALGL

SEQ ID NO: 249 - protein, 585 aa, Artificial Sequence
  Synthetic
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 250 - protein, 585 aa, Artificial Sequence
  Synthetic
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDKFAAFVEKCCKADDKETCFAEEGPKLVAASKAALGL

SEQ ID NO: 251 - protein, 585 aa, Artificial Sequence
  Synthetic
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDDFAAFVEKCCKADDKETCFAEEGPKLVAASKAALGL

SEQ ID NO: 252 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 253 - protein, 333 aa, Artificial Sequence
  Synthetic
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 254 - protein, 330 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 255 - protein, 338 aa, Artificial Sequence
  Synthetic
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 256 - protein, 227 aa, Artificial Sequence
  Synthetic
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 257 - protein, 227 aa, Artificial Sequence
  Synthetic
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 258 - protein, 232 aa, Artificial Sequence
  Synthetic
EPKSSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 259 - protein, 232 aa, Artificial Sequence
  Synthetic
EPKSSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 260 - protein, 232 aa, Artificial Sequence
  Synthetic
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 261 - protein, 232 aa, Artificial Sequence
  Synthetic
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 262 - protein, 217 aa, Artificial Sequence
  Synthetic
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVTTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 263 - protein, 227 aa, Artificial Sequence
  Synthetic
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 264 - protein, 225 aa, Artificial Sequence
  Synthetic
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 265 - protein, 227 aa, Artificial Sequence
  Synthetic
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 266 - protein, 225 aa, Artificial Sequence
  Synthetic
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 267 - protein, 227 aa, Artificial Sequence
  Synthetic
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 268 - protein, 225 aa, Artificial Sequence
  Synthetic
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 269 - protein, 227 aa, Artificial Sequence
  Synthetic
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 270 - protein, 225 aa, Artificial Sequence
  Synthetic
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 271 - protein, 484 aa, Artificial Sequence
  Synthetic
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 272 - protein, 480 aa, Artificial Sequence
  Synthetic
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 273 - protein, 484 aa, Artificial Sequence
  Synthetic
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 274 - protein, 480 aa, Artificial Sequence
  Synthetic
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 275 - protein, 484 aa, Artificial Sequence
  Synthetic
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 276 - protein, 480 aa, Artificial Sequence
  Synthetic
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 277 - protein, 484 aa, Artificial Sequence
  Synthetic
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 278 - protein, 480 aa, Artificial Sequence
  Synthetic
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 279 - protein, 5 aa, Artificial Sequence
  Synthetic
HHHHH

SEQ ID NO: 280 - protein, 6 aa, Artificial Sequence
  Synthetic
HHHHHH

SEQ ID NO: 281 - protein, 502 aa, Artificial Sequence
  Synthetic
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 282 - protein, 502 aa, Artificial Sequence
  Synthetic
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 283 - protein, 508 aa, Artificial Sequence
  Synthetic
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 284 - protein, 508 aa, Artificial Sequence
  Synthetic
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 285 - protein, 6 aa, Artificial Sequence
  Synthetic
DTYRYI

SEQ ID NO: 286 - protein, 6 aa, Artificial Sequence
  Synthetic
TDFYLK

SEQ ID NO: 287 - protein, 11 aa, Artificial Sequence
  Synthetic
MASMTGGQQMG

SEQ ID NO: 288 - protein, 14 aa, Artificial Sequence
  Synthetic
GKPIPNPLLGLDST

SEQ ID NO: 289 - protein, 6 aa, Artificial Sequence
  Synthetic
QYPALT

SEQ ID NO: 290 - protein, 10 aa, Artificial Sequence
  Synthetic
SSTSSDFRDR

SEQ ID NO: 291 - protein, 7 aa, Artificial Sequence
  Synthetic
DYKDDDK

SEQ ID NO: 292 - protein, 6 aa, Artificial Sequence
  Synthetic
EYMPME

SEQ ID NO: 293 - protein, 6 aa, Artificial Sequence
  Synthetic
EFMPME

SEQ ID NO: 294 - protein, 19 aa, Artificial Sequence
  Synthetic
KDHLIHNVHKEFHAHAHNK

SEQ ID NO: 295 - protein, 8 aa, Artificial Sequence
  Synthetic
QPELAPED

SEQ ID NO: 296 - protein, 11 aa, Artificial Sequence
  Synthetic
KPPTPPPEPET

SEQ ID NO: 297 - protein, 11 aa, Artificial Sequence
  Synthetic
CEQKLISEEDL

SEQ ID NO: 298 - protein, 7 aa, Artificial Sequence
  Synthetic
HHHHHHH

SEQ ID NO: 299 - protein, 8 aa, Artificial Sequence
  Synthetic
HHHHHHHH

SEQ ID NO: 300 - protein, 9 aa, Artificial Sequence
  Synthetic
NANNPDWDF

SEQ ID NO: 301 - protein, 15 aa, Artificial Sequence
  Synthetic
KETAAAKFERQHMDS

SEQ ID NO: 302 - protein, 8 aa, Artificial Sequence
  Synthetic
WSHPQFEK

SEQ ID NO: 303 - protein, 9 aa, Artificial Sequence
  Synthetic
AWAHPQPGG

SEQ ID NO: 304 - protein, 6 aa, Artificial Sequence
  Synthetic
HTTPHH

SEQ ID NO: 305 - protein, 11 aa, Artificial Sequence
  Synthetic
YTDIEMNRLGK

SEQ ID NO: 306 - protein, 12 aa, Artificial Sequence
  Synthetic
EDQVDPRLIDGK

SEQ ID NO: 307 - protein, 5 aa, Artificial Sequence
  Synthetic
QDGNE